(19) United States
(12) Patent Application Publication
(10) Pub. No.: US 2017/0233698 A1
(43) Pub. Date: Aug. 17, 2017

(54) CULTURED MAMMALIAN LIMBAL STEM CELLS, METHODS FOR GENERATING THE SAME, AND USES THEREOF

(71) Applicant: THE REGENTS OF THE UNIVERSITY OF CALIFORNIA, Oakland, CA (US)

(72) Inventors: Kang Zhang, La Jolla, CA (US); Hong Quyang, La Jolla, CA (US); Rui Hou, Shenyang (CN); Huimin Cai, Shenyang (CN)

(21) Appl. No.: 15/322,423

(22) PCT Filed: Jun. 29, 2015
(86) PCT No.: PCT/US15/38384

§ 371 Date: Dec. 27, 2016

Related U.S. Application Data
(60) Provisional application No. 62/018,396, filed on Jun. 27, 2014.

(51) Int. Cl. C12N 5/079; A61K 35/36; A61K 35/30; C12N 5/071; C12Q 1/68

(52) U.S. Cl. CPC C12N 5/0621; C12N 5/0625; C12Q 1/6883; A61K 35/30; A61K 35/36; C12N 2506/03; C12N 2533/90; C12N 2510/00; C12N 2501/235; C12N 2501/998; C12N 2509/00; C12Q 2600/106

(57) ABSTRACT

The invention provides an isolated limbal stem or progenitor cell (LSC) population or LSC-like population comprising a chemically synthesized, recombinant or isolated nucleic acid encoding PAX6 integrated into a chromosome, or alternatively, not integrated remaining as an extrachromosomal genetic material, wherein the isolated LSC population is substantially free of non-LSC cells or wherein the LSC-like population is substantially free of non-LSC-like cells, or wherein the isolated LSC or LSC-like population is substantially free of non-LSC and non-LSC-like cells and uses thereof.

This application claims the benefit of U.S. Provisional Application No. 62/018,396, filed Jun. 27, 2014, which is incorporated herein by reference in its entirety.

Throughout this application various publications are referenced. The disclosures of these publications in their entireties are hereby incorporated by reference into this application in order to more fully describe the state of the art to which this invention pertains.

### FIELD OF THE INVENTION

The field of the invention is directed to methods and compositions for treating ophthalmic disorders, diseases and injuries. In particular, the field of the invention is directed to methods, kits and compositions for treating disorders, diseases, defects and injuries of the cornea and ocular surface. The present disclosure relates to preparations of cultured mammalian limbal stem cells, derived from corneal limbus tissue. In preferred embodiments, the limbal stem cell lines are self-renewing and have the ability to differentiate into corneal epithelial tissues. Methods for culturing limbal stem cell lines and methods and compositions of their use are also disclosed.

### BACKGROUND OF THE INVENTION

It is known that adult stem cells are present in the comeoscleral limbus of the eye. These cells participate in the dynamic equilibrium of the corneal surface and replace superficial epithelial cells that are shed and sloughed off during eye-blinking. Severe damage to the limbal stem cells from chemical or thermal burns, contact lenses, severe microbial infection, multiple surgical procedures, cryotherapy, or diseases such as Steven-Johnson syndrome or ocular cicatrical pemphigoid can lead to destruction of limbal stem cells and limbal stem cell deficiency which can lead to an abnormal corneal surface, photophobia, and reduced vision (Anderson et al., (2001) Br. J. Opthalmol. 85:567-575). This damage cannot be repaired without the re-introduction of a source of limbal stem cells (Tseng et al., (1998) Arch. Opthalmol. 116:431-41; Tsai et al., (2000) N. Engl. J. Med. 343:86-93; Henderson et al., (2001) Br. J. Opthalmol. 85:604-609). Thus, limbal stem cells, with their high proliferative capacity, are clearly crucial for the maintenance of a viable ocular surface, because they provide an unbroken supply of corneal epithelial cells necessary to maintain the equilibrium of the corneal surface (Tseng, (1996) Mol. Biol. Rep. 23:47-58).

Several approaches have been used to attempt to restore normal vision after corneal surface impairments, however these approaches are generally not sufficient to repair damage related to or resulting from the loss of limbal stem cells. One conventional approach is to repair a damaged corneal surface by transplanting amniotic membrane directly onto the surface of the subject's eye. (Anderson et al., (2001) Br J. Opthalmol. 85:567-575). Amniotic membrane transplantation has been found to facilitate epithelization, maintain a normal epithelial phenotype, reduce inflammation, reduce scarring, reduce adhesion of tissue, and reduce vascularization in the eye. Amniotic membrane transplantations, however, have the disadvantage of not being uniformly successful, with the final outcome often not much different than the patient's starting point (Prabhasawat et al., (1997) Arch. Ophthalmol 115:1360-67). Methods for isolating human amniotic epithelial cells and differentiating them into corneal surface epithelium are also disclosed by Hu et al (WO 00/73421).

Another approach to treating corneal damage involves corneal transplantation. (Lindstrom, (1986) N. Engl. J. Med. 315:57-59). Still another approach to treating limbal stem cell deficiency is to transplant limbal grafts from a donor eye into a recipient eye.

Given the drawbacks of removing large limbal biopsies from a living donor, other methods have been developed for treating limbal stem cell deficiencies which rely on taking only a small biopsy of limbal epithelium from the healthy eye (Pellegrini et al., (1997) Lancet 349:990-993), (Koizumi et al., (2000) Invest. Ophthalmol. Vis. Sci. 41:2506-2513; Koizumi et al., (2000) Cornea 19:65-71; Dua et al., (2000) Surv. Ophthalmol. 44:415-425; Jun Shimazaki et al., (2002) Opthalmol. 109:1285-1290). Another approach utilizing amniotic membranes is disclosed in U.S. Publication No. 20030208266, which describes the transplantation of epithelial stem cells that are cultured ex vivo on specifically treated amniotic membrane.

Another approach for generating grafts is set forth in EP Patent No. 0572364, which discloses the process of growing biopsies of human eye surface epithelium in vitro, with the biopsies derived from the limbus and/or perlimbus area of the eye, or the forrinx and/or conjunctiva area of the eye. Another patent application, WO 03/030959, discloses a corneal repair device for treating corneal lesions that uses a contact lens with a modified surface for culturing limbal stem cells.

Similarly, U.S. Publication No. 20020039788 discloses a bioengineered composite graft for the treatment of damaged or diseased corneal epithelial surfaces, wherein the composite graft comprises a multilayered epithelium of differentiated epithelial cells. U.S. Pat. No. 6,610,538 discloses methods of reconstructing laminae of human epithelium comeae in vitro from cultures of limbal stem cells to use as grafts for patients with ocular damage. WO 03/093457 also discloses a method for the identification and isolation of stem cells from corneal tissue by means of selecting stem cells that express the membrane protein markers CD34 or CD133, both of which belong to the differentiation cluster (CD).

The stability and success of any limbal cell transplant depends on its ability to regenerate continuously the viable limbal stem cells for repopulating the ocular surface. The transplants or grafts currently used to treat limbal stem cell deficiencies generally contain high percentages of differentiated corneal epithelial cells rather than limbal stem cells, which may be present in only limited amounts. The donor epithelium in such transplants or grafts will survive generally for only a short period of time due to the limited supply of limbal stem cells. Alternatively, the transplants may yield a clear corneal epithelium, but the lack of sufficient limbal stem cells results in abnormal epithelial surfaces and poor healing, resulting in a failure to repair the ocular surface and improve vision. Therefore, these approaches which are intended to supply limbal stem cells to an eye with a limbal stem cell deficiency have serious limitations, which may be due to the limited supply of undifferentiated limbal stem cells with self-regenerative capacity present in the transplants or grafts. Thus, it is desirable to provide transplants or grafts that will be more successful in repairing and reconstructing ocular surface impairments by providing sufficient populations of limbal stem cells with the ability to regenerate and continually supply limbal stem cells to the eye.

To date, no treatment option exists that is able to completely ameliorate corneal epithelial damage, or induce the growth and development of new corneal epithelial cells or limbal stem cells to replace damaged or dead cells, any or all of which could help return the patient to normal or near normal visual function. Therefore, it is an object of the instant invention to provide such treatment options for patients suffering from ophthalmic disorders, diseases and injuries, in particular, corneal disorders, diseases and injuries.

### BRIEF SUMMARY OF THE INVENTION

The present disclosure describes the culturing of mammalian limbal stem cells (LSCs) derived from non-embryonic tissue, preferably corneal limbal tissue. In particular, the present disclosure provides cultured mammalian LSCs, and methods of generating cultured mammalian LSCs, which: (i) are isolated from corneoscleral limbus, (ii) expanded in an in vitro culture, and (iii) maintain the potential to differentiate into lineage-committed corneal epithelial cells in an in vitro culture or therapeutically on the eye of a subject in need thereof.

In certain embodiments, the LSCs are cultured cells cultured in a feeder-free culture media on an extracellular matrix, further supplemented with a cell media comprising at least a minimum essential medium plus optional agents such as growth factors, serum, and one or more soluble factors.

The limbal stem cells of the present invention can be isolated from any suitable mammal. In some embodiments, the limbal stem cells of the present invention can be isolated from a donor who is not a recipient. Such donors can be cadavers or organ donors who are biocompatible with the subject. In some embodiments, the limbal stem cells of the present invention can be isolated from a donor who is also a recipient, such that the recipient and the donor can be the same individual or subject.

Isolation of limbal stem cells can be made prior to, during or after culture and expansion. In a preferred embodiment, dissociated tissues can be utilized to isolate LSC of the invention which are then expanded.

The limbal stem cells of the invention can be cultured in culture media that supports the growth and expansion of limbal stem cells. The isolated LSCs remain substantially undifferentiated in an in vitro culture for a multitude of passages.

In certain embodiments, the limbal stem cells of the invention are cultured on an appropriate support material such as an extracellular matrix or biocoated surface, for example extracellular matrix carrier or biocoated lens. The surface material may be any support biocoated with one or more attachment factors as described herein.

The limbal stem cells of the invention may be utilized in various therapeutic manners, for example, in a method for treating an ophthalmic disorder, disease or injury in a patient in need thereof, comprising administering to the patient a therapeutically effective amount of one or more compositions comprising isolated LSC cells. For example, the present invention contemplates methods for stimulating proliferation or regeneration of corneal epithelial cells in a patient in need thereof, or a patient with limbal stem cell deficiency.

In one embodiment of the present invention, a subject is presented with LSC cells of the invention on an appropriate support material, such as an extracellular matrix or biocoated surface. The support material can be de novo or the support material utilized for culturing the LSC of the invention. For example, an exemplary of the present invention comprises cultured LSC on a biocoated lens kit. In an alternate embodiment, LSC of the invention are isolated from culture media and presented to a subject in need thereof, with an extracellular matrix, media and other materials. Similarly, media and other factors can be derived from the culture media. In one example, LSC of the invention may be administered in combination with other agents or treatment modalities. In more specific embodiments, the other agents are active agents. And in the most specific embodiments, the active agents include growth factors, cytokines, inhibitors, immunosuppressive agents, steroids, chemokines, antibodies, antibiotics, antifungals, antivirals, mitomycin C, or other cell types. Another specific embodiment is one in which the other treatment modalities include contact lens, drops, and other ophthalmic means for delivering LSC.

The present invention provides LSCs and materials for preparing the material for repair of the cornea, comprising the steps of: (1) isolating limbal stem cells, from a limbal stem cell suspension; (2) The steps (1) above including obtaining limbal stem cell from the suspension and seeding on a scaffold placed in inducing culture medium to promote limbal stem cells to differentiate into corneal epithelial cells.

In step (1) above, in one embodiment, the limbal stem cells may be obtained by the following method: Fresh limbal tissues may be cleaned, cut into small pieces, and treated with 0.2% collagenase IV at 37° C. for 2-4 hours to digest cell mass. Limbal tissue may be further digested by 0.25% trypsin and 1 mM EDTA for 10 to 20 minutes at a single cell suspension at 37° C. in 1-3% Matrigel coated culture dish.

Preferably, in another embodiment, collagenase IV digestion time was 3 hours; 0.25% trypsin and 1 mM EDTA digestion time was 15 minutes; Matrigel concentration was 2%.

Further, in yet another embodiment, in the limbal stem cell suspension, the cell concentration may be about 2×10²˜8×10²/ul.

Additionally, in an embodiment of the invention, in step (2), the scaffold may be a material of biological origin, or a cell-free lens.

In yet a further embodiment, in step (2), the biological material may be a cell-derived collagen or Matrigel amnion.

Additionally, in one embodiment, in step (2), the medium used for induction is epithelial cell culture medium CnT-30.

In an additional embodiment, in step (2), the culture time for the induction culture may be from about 3 to 18 days. Preferably, in step (2), the culture time is about 14˜18 days.

The present invention also provides the above treatment of corneal repair materials for the preparation of a medicament for corneal injury.

Other features and advantages of the invention will be apparent from the accompanying description, examples and the claims. The contents of all references, pending patent applications and issued patents, cited throughout this application are hereby expressly incorporated by reference.

### DESCRIPTION OF THE FIGURES

FIG. 1a-d. Normal and pathological changes of corneal epithelium, and its comparison to skin. a, Normal cornea-limbus junction. Limbus identified by K19 and P63 (also see FIG. 2e) and cornea by K12. b, Normal skin epidermis identified by p63 and K5/K14 (see FIG. 2a, b) in the basal layer and absence of K3/K12. c, Normal central cornea labeled by K3/K12 and absence of p63 and K1/K10 (also see FIG. 2c, d, f). d, Cornea with abnormal epidermal differentiation showing absence of K3/K12 (a′) and presence of skin epithelium makers p63 (b′) and K5/K1/K10 (c′-e′). H&E stain, left panels. Scale bar: 100 Atm.

FIG. 2a-j. Keratin expression profiles and cell cultures/3-D differentiation of LSCs and SESCs. a-f, Keratin expression profiles in human limbus, cornea and skin epidermis. a, b, Peripheral cornea-limbus junction and skin tissues showing positive K5 (a) and K14 (b) expression in the basal cell layer of limbus and skin, and their absence in central corneal epithelium. c-d, Skin epidermis showing positive K1 (c) and K10 (d) expression and their absence in cornea and limbus. e-f, Peripheral cornea-limbus junction showing positive K19 in limbus and negative in central corneal epithelium and skin (e), and positive K3/K12 only in cornea and negative in limbus and skin (f). g-j, Cultured LSCs with stem/progenitor cell and SESCs characteristics at passage 12 and validation of a 3-D differentiation system. g, Immunofluorescence staining of LSCs showing positive stem cell signals of p63 (a′) and Ki67 (b′) and negative differentiated CEC signals, K3/12 (c′), phase contrast photograph (d′); (h) qPCR analysis showing K3/K12 up-regulation and K19 down-regulation in CECs from a 3-D differentiation assay compared with LSCs; i, K1/K10 up-regulation in SECs from 3-D differentiation assay compared with SESCs (c), all n=3, p<0.01. j, Immunofluorescence staining of cultured SESCs showing positive p63 (a′) and negative signals for limbus stem cell marker, K19 (b′) and mature skin epithelium markers K1/K10 (c′, d′). Scale bar, 100 μm.

FIG. 3a-f. Exclusive expression of WNT7A and PAX6 at limbus and cornea. a-d, Immunofluorescence staining of cultured LSCs and SESCs and 3-D differentiated CECs and SECs. Left panels, phase contrast photographs; staining of p63, K19 and Ki67 in LSCs (a), p63, K5 and Ki67 in SESCs (c). K3/12, K1, K5, K10 and K14 in CECs (b) and SECs (d) in 3-D culture spheres. e, Heatmap depicting differential gene expression comparing among LSCs, CECs and SESCs. * denotes WNT7A and PAX6. f, Immunofluorescence staining of WNT7A and PAX6 at limbus, cornea and skin (a′-d′). Expression of WNT7A and PAX6 in cultured LSCs (e′, g′) and 3-D CEC spheres (f′, h′). Abbreviations: LSCs: limbal stem/progenitor cells, SESCs, skin epithelial stem cells, CECs, corneal epithelial cells, SECs, skin epithelial cells. Scale bar, 100 μm.

FIG. 4a-i. Gene expression analysis. a-c, Genome wide gene expression microarray of LSCs, CECs and SESCs. a, The top 100 significant genes from comparing LSCs/CECs to SESCs. b, Validation of the microarray data with qPCR analysis showing a strong correlation. c, qPCR analysis of WNT7A and PAX6 expression in LSCs and CECs compared to SESCs, all n=3, p<0.05. d, Expression of WNT7A and PAX6 in cornea and limbus of a one-year old human infant. H&E stain (a′), boxed area was shown in serial sections (b′-d′) with immunofluorescence staining of WNT7A (b′), PAX6 (c′) and K3/12 (d′). Scale bar, 100 μm

FIG. 5a-c. WNT7A and PAX6 are essential for maintenance of cornea cell fate. a, Human corneal epithelium squamous metaplasia. Red box (left box of pair; or single large box in a′) indicates an area of metaplasia, blue box (right box of pair) indicates an area of relatively normal cornea; Top panel: H&E stain (top panel) showing typical skin epidermal morphology with positive p63 at basal layer (a′, arrowheads indicate p63 staining), loss of WNT7A (b′) and PAX6 (c′) were accompanied by absence of corneal K3/K12 (d′). Serial sections of the areas marked by red and blue boxes in the top panel are represented in the lower panels. b, Immunofluorescence of 3-D differentiated cells with WNT7A or PAX6 knockdown; K1 and K5, left panels; PAX6 and K10, middle panels; K3/12. right panels; c, Quantitative PCR analysis of gene expression changes of cornea or skin epithelium markers in 3-D differentiated cells with WNT7A or PAX6 knockdown (all n=3, p<0.05). Data are shown as means±s.d. Scale bar, 100 μm.

FIG. 6a-e. Appearance of skin epidermal markers with loss of corneal markers in human corneal diseases. Appearance of skin epidermal marker p63, K5 and K10 with loss of corneal marker K3/12. PAX6 and WNT7A in cornea of patients with Steven-Johnson syndrome (a, b), ocular pemphigoid (c), trauma injury (d) and alkaline burn (e). For all images, H&E staining was carried out on the lesion of corneal epithelial squamous metaplasia (a′). b′-f, the same region of lesion in serial sections showing increased p63 (b′, d′) and K5 (c′, d′) and K10 (e′) in the suprabasal layer, no WNT7A (e′), K3/12 or PAX6 could be detected in the area (f′). Scale bar, 100 μm.

FIG. 7a-f. The effect of WNT7A/FZD5 on PAX6 expression in LSCs. a-c, The effect of WNT7A knockdown on PAX6 expression in LSCs. a, phase contrast photographs showing effects of HNT7A and PAX6 knockdowns (shWNT7A and shPAX6) in LSCs and their 3-D differentiation spheres. b, qPCR analysis of gene expression changes of WNT7A and PAX6 in LSCs. WNT7A knockdown decreased PAX6 expression (n=3, p<0.01); no significant change in WNT7A expression in PAX6 knockdown. c, Validation of knockdown efficiency of WNT7A and PAX6 in LSCs by western blot analysis. d-f, WNT7A and FZD5 acted as the upstream regulators of PAX6 expression. d, phase contrast photographs showing cell morphology of knockdown of FZD5 (shFZD5) in LSCs and 3-D differentiation spheres. e, Co-immunoprecipitation of WNT7A and FZD5 in LSCs. f, qPCR analysis of gene expression changes in corneal and skin epithelial markers in 3-D differentiated cells of LSCs with FZD5 knockdown (3-D shFZD5 LSCs). FZD5 knockdown did not affect WNT7A expression; all others, n=3, p<0.05. Scale bar, 100 μm.

FIG. 8a-c. The effect of PAX6 transduction in SESCs. a, Phase contrast photographs of SESCs with PAX6 transduction (PAX6) and 3-D differentiation spheres. b, Validation of K12 and PAX6 expression in 3-D differentiation spheres by western blotting analysis. c, Loss of skin-specific keratins, K1/K10 in 3-D differentiation of SESCs with PAX6 transduction (3-D PAX6⁺ SESCs). Scale bar, 100 μm.

FIG. 9a-e. Conversion of SESCs into corneal epithelial-like cells by PAX6 transduction. a, Double immunofluorescence staining of PAX6 and p63 in transfected SESCs, K19 was positive in PAX6-transduced (PAX6) SESCs. b, Immunofluorescence staining of K3/12 and PAX6⁺ SESCs in 3-D differentiation conditions. c, qPCR analysis of gene expression of keratins in PAX6⁺ SESCs (all n=3, p<0.05). Data are shown as means±s.d. d, Hierarchical cluster analysis among CECs, differentiated LSCs with PAX6 knockdown (3-D shPAX6 LSCs), SECs and differentiated SESCs with PAX6 transduction (3-D PAX6⁺ SESCs). e, Schematic diagram showing normal LSCs differentiation into CECs (a′) and proposed mechanism in which loss of WNT7A/PAX6 in LSCs leads to abnormal skin epidermis-like differentiation in corneal surface epithelial cell disease (b′). Scale bar, 100 μm.

FIG. 10a-c. Quantitative Information of RNA-seq data. a, Statistical analysis of RNA-seq samples: raw reads, mapping reads and mapping rate of each sample are included. b, Pairwise comparisons of duplicated biological samples. c, The differences between SECs and 3-D PAX6 SESCs, CECs and 3-D shPAX6 LSCs, all FDR<0.001. a, qPCR analysis of PAX6 expression in rabbit SESCs with PAX6 transduction (Rb PAX6⁺ SESCs) or LSCs with PAX6 knockdown (Rb shPAX6 LSCs) (all n=3, p<0.05). We noticed some minor differences in the heatmap, which might result from some experimental variations or were due to the possibility that, while PAX6 expression is largely responsible for cell fate switch from SESCs to CECs at both the gene expression and functional levels as we demonstrated in the current study, this single transcription factor may not be sufficient to create cells that are completely identical to CECs.

FIG. 11a-f. Engineered expression of PAX6 and rabbit LSC deficiency model. a-e, Quantification and culture of engineered expression of PAX6 in rabbit SESCs and PAX6 knockdown LSCs. a, qPCR analysis of PAX6 expression in rabbit SESCs with PAX6 transduction (Rb PAX6⁺ SESCs) or LSCs with PAX6 knockdown (Rb shPAX6 LSCs) (all n=3, p<0.05). b, Rabbit SESCs with positive staining of p63 and negative staining of PAX6. Left panel, phase contrast photograph. c, Upper row, double immunofluorescence staining of PAX6 and p63 in rabbit SESCs with PAX6 transduction. Upper left panel, phase contrast photograph. Bottom row, rabbit PAX6⁺ SESCs were further labeled with GFP for transplantation. d, Rabbit LSCs with positive staining of p63 and PAX6. Upper left panel, phase contrast photograph. e, Culture of GFP-labeled rabbit LSCs with PAX6 knockdown. f, a′, Conjunctiva peritomy was performed and a circumferential strip of 2 mm anterior limbal conjunctiva was removed. b′-d′, lamellar scleral and corneal dissection to completely remove LSCs and corneal epithelium along an anterior cornea stroma plane. Dissected cap is shown in (d′, arrows). e′-f′, the exposed cornea stroma bed was covered by human amniotic membrane (e′) and sutures (f). (n=3). Scale bars, 100 μm.

FIG. 12a-d. Cornea epithelium regeneration and repair by transplanted GFP-labeled PAX6⁺ SESCs on a rabbit LSC deficiency model, a, Time course of corneal epithelial defect repair. 15 days post transplantation: decreased cornea clarity with an entire corneal epithelial defect evidenced by fluorescein stain of cornea surface; 30 days post transplantation, improved cornea clarity and reduced fluorescein staining of cornea epithelial defect; 45 and 90 days post transplantation: restoration and maintenance of cornea clarity. b-c, other two examples of regeneration and repair of rabbit corneal epithelial surface 90d post transplantation with GFP-labeled PAX6⁺ SESCs showing complete repair and re-epithelization of corneal epithelial defects. a-c, panels from left: white light micrograph, slit-lamp micrograph and fluorescein staining (note: bright spots on corneal surface were due to camera light reflection, they were not epithelial defects) of corneal epithelium. (n=5). d, H&E stain of regeneration and repair of corneal epithelial surface in three separate rabbits 90d post transplantation with GFP-labeled PAX6 SESCs showing intact corneal epithelium histology.

FIG. 13a-c. Corneal epithelial regeneration by transplantation in a rabbit LSC deficiency model, a, Time course of corneal epithelial regeneration and repair on rabbit LSC deficiency model post transplantation with GFP-labeled PAX6⁺ SESCs. Upper panels, 3 days post transplantation. Left, light micrograph showing a hazy cornea; right, GFP+ donor cells at limbal region (arrows). Lower panels, 20 days post transplantation. Left, light micrograph showing a cornea with partial clarity; right, GFP⁺ donor co-located in transparent areas (arrows). Scale bar, 1 mm. We observed that only the transplanted cells from limbal region could survive, proliferate, and regenerate cornea surface epithelium, suggesting that limbus contained stem cell niche favorable for stem cell survival and growth. b, Culture and re-isolation of reprogrammed donor GFP-labeled PAX6⁺ SESCs epithelial cells from the limbal region of a rabbit recipient eye 90 days post transplantation with GFP-labeled PAX6⁺ SESCs. Upper panel, double immunofluorescence staining of PAX6 and GFP; bottom panel, double immunofluorescence staining of p63 and GFP in PAX6-transduced rabbit SESCs. Scale bars, 100 μm. c, Repair and recovery of a repeat cornea epithelium injury on a cornea transplanted with GFP-labeled PAX6⁺ SESCs. Upper panels, we iatrogenically scraped and removed donor-derived corneal epithelial cells and made a large corneal surface epithelium defect (arrows) 3 months post initial transplantation of PAX6⁺ SESCs. Bottom panels, complete repair and recovery were observed within 72 h with healed epithelial defect (n=3). Left panels, light micrographs; middle panels, slit-lamp micrographs; right panels, fluorescein staining.

FIG. 14a-g. Cell transplantation and cornea epithelium repair on a rabbit limbal stem cell deficiency model, a, Immunofluorescence staining of rabbit corneas two month post transplantation. Upper panels, cornea transplanted with GFP-labeled PAX6⁺ SESCs, showing positive GFP signals and the expression of the corneal epithelium markers K3 and K12 on the corneal surface. Bottom panels, cornea transplanted with GFP-labeled shPAX6-LSCs, showing positive GFP signals and the expression of the skin epidermal epithelium marker K10. Scale bar, 100 μm. b-f, Rabbit corneas 2-month post cell transplantation (left panels, H&E stain; central two panels, white light micrograph and slit-lamp micrograph; right panels, fluorescein dye staining of corneal epithelium surface). Scale bars, 100 μm. b, Normal cornea with typical corneal epithelium histology and intact cornea surface without epithelial defects. c, Denuded cornea covered with a human amniotic membrane only, showing histology of epithelial metaplasia and opaque cornea with vascularization (n=4). d-e, Cornea transplanted with GFP-labeled LSCs (d, n=3) and GFP-labeled PAX6⁺ SESCs (e, n=5), showing corneal epithelium histology, healed and intact cornea surface without epithelial defects. f, Cornea transplanted with GFP-labeled, shPAX6-treated LSCs, showing histology of epithelial metaplasia, opaque and vascularized corneal surface with epithelial defects (n=4). g, Rabbit cornea 3 months post transplantation with GFP-labeled PAX6⁺ SESCs: smooth, transparent cornea (top panel) with positive GFP signals (second panel, scale bar, 1 mm). The framed area in the second panel is enlarged to show the expression of GFP (middle panel), PAX6 (fourth panel) and both GFP-PAX6 (bottom panel). Scale bars, 100 μm.

FIG. 15a-e. Pax6 and p63 expression pattern of mouse cornea at different embryonic stages. A, positive PAX6 at E12.5 in mouse cornea. p63 is undetectable. Higher magnification (left panel) shows corneal-limbus-conjunctival junction areas (red box) stained with hematoxylin and eosin (H&E). B, PAX6 expression is marked in ocular areas (conjunctival, limbal and corneal tissues, white arrows), whereas p63 was positive in eyelid skin, limbus and cornea at E14.5. The left panel shows H&E stain. C and D, PAX6 and p63 expression in mouse cornea at E16.5 (C) and E18.5 (D). Right upper panels, PAX6 and p63 stain of the area depicted in the red box in the H&E stain (left panel) showing PAX6 expression in all ocular tissues and p63 expression in cornea tissues; right lower panels, higher magnifications of the areas framed by the yellow boxes. E, lineage tracing of PAX6 in the corneal epithelium of ROSA^{mT/mG}; PAX6-GFPcre mice at P1 and P60, ROSA^{m1/mG} serve as a control. Scale bars=100 μm.

FIG. 16a-b. Characterization of human corneal and skin epithelia. A, cornea identified by PAX6 and K3/12 and absence of p63, K5, K1 and K10. B, skin epidermis identified by p63, K5, K1 and K10 and absence of PAX6 and K3/12. Left upper panels, H&E stain. Scale bars=100 μm.

FIG. 17a-c. Immunofluorescene staining of human limbus area and cultured human LSCs and SESCs. A, human limbus region identified by PAX and p63. Left upper panel, H&E stain of the cornea-limbus junction (arrow). B, cultured LSCs stained with PAX6 and p63. Left panel, phase contrast. C, cultured SESCs stained with p63 and K5. Left panel, phase contrast. Scale bars=100 μm.

FIG. 18a-b. PAX6 is essential for maintenance of cornea cell fate. A, phase contrast showing cell morphology and Ki67 staining of PAX6 knockdown in human LSCs and their differentiated cells. B, quantitative PCR analysis of gene expression changes in cornea or skin epithelial markers in differentiated PAX6 shRNA LSCs (shPAX6-LSCs) compared with differentiated LSCs (n=3, p<0.05). Data are shown as means±S.D. Scale bars=100 μm.

FIG. 19a-b. Appearance of skin epidermal markers with loss of corneal markers in patients with corneal-limbal dermoid. A, patient with a typical corneal limbal dermoid (panel a) and H&E stain (panel b). B, serial sections showing increased p63 (panel a) and K5 (panel b) and K10 (panel c) in the suprabasal layer. No K3/12 or PAX6 could be detected (panel d). Scale bars=100 μm.

FIG. 20a-c. Identified signal pathways involved in LSCs and SESCs. A, heat maps of gene expression data in Wnt, Notch and TGF-β pathways with comparison of LSCs and SESCs. For each gene in a heat map, red and blue denote high and low expression, respectively, relative to the average expression level in all samples. B, graphical representation of genetic interactions between genes belonging to the Wnt and Notch pathways. The fold difference between average expression values in two independent LSC and SESC preparations was used to color-code each gene individually (red, higher expression in LSCs; blue, higher in SESCs). Subsets of genes associated with the Notch and Wnt pathways were selected.

### DETAILED DESCRIPTION OF THE INVENTION

### Definitions

The term “culture medium,” “cell culture medium,” or “cell medium” is used to describe a cellular growth medium in which cells are grown, for example, stem cells, progenitor cells, or differentiated cells. Culture medium is well known in the art and comprises at least a minimum essential medium plus optional agents such as growth factors (e.g., including fibroblast growth factor, preferably basic fibroblast growth factor (bFGF), and epidermal growth factor (EGF)), cytokines (e.g., leukaemia inhibition factor (LIF)), hormones (e.g., including glucocorticoids (such as hydrocortisone) and thyroid hormone (such as, 3,3′,5-triiodo-L-thyronine)), glucose, non-essential amino acids, glutamine, insulin, transferrin, beta mercaptoethanol, ROCK inhibitors, cholera toxin, and other agents well known in the art. Such media include commercially available media such as DMEM/F12 (1:1), which may be supplemented with any one or more of L-glutamine, knockout serum replacement (KSR), fetal bovine serum (FBS), non-essential amino acids, leukemia inhibitory factor (LIF), epidermal growth factor (EGF), beta-mercaptoethanol, basic fibroblast growth factor (bFGF), hydrocortisone, 3,3′,5-triiodo-L-thyronine, a ROCK inhibitor, an antibiotic, B27 medium supplement and/or other medium supplement. Cell media useful in the present invention are commercially available and can be supplemented with commercially available components, available from Invitrogen Corp. (GIBCO) and Biological Industries, Beth HaEmek, Israel, among numerous other commercial sources.

A “LSC culture medium” or “LSC maintenance medium” is a culture medium formulated for in vitro and stable proliferation of limbal stem or progenitor cell (LSC) or LSC-like cells.

A “differentiation medium” is a culture medium or cell culture medium which is formulated for in vitro differentiation of a stem or progenitor cell into cells of a particular cell lineage. For example, a “LSC differentiation medium” may be a culture medium that is formulated for in vitro differentiation of limbal stem or progenitor cells or LSC-like cells to corneal epithelial cells (CECs) or CEC-like cells.

A “feeder-free culture medium” refers to the fact that the culture medium used to culture the cells of interest, i.e., LSC or SECS, do not need feeder cells in order to permit the cells to proliferate stably. For example, LSC cells cultured in “feeder-free culture medium” in which no feeder cell layer is present in the culture are able to divide and be maintained as LSC.

“Serum-free” refers to a lack of serum, which is a clarified blood product obtained from an animal or human.

A “serum-free” culture medium is a culture medium lacking serum. It may contain, for example, serum substitute or serum replacement.

A “chemically defined” medium or “chemically defined” culture medium is a culture medium whose components are chemically defined. As such, it lacks serum, a chemically undefined component. For culturing of cells or tissues requiring serum, a “chemically defined” medium typically contains serum substitute or serum replacement in place of serum. A “chemically defined” medium is “xeno-free” if it contains no animal-derived product or no foreign animal derived product. It may also be free of human-derived product. It is generally desirable to replace animal- or human-derived products with recombinantly produced materials, chemically synthesized materials, or enzymatically synthesized materials, which have no prior animal contact or exposure.

“Stem cells” are cells that exhibit self-renewal, give rise to progenitor cells, which can proliferate and differentiate to terminally differentiated cells, which are post-mitotic. For example, limbal stem cells can divide to produce limbal stem cells as well as progenitor cells. Progenitor cells can be directed to undergo differentiation (through for example culturing in vitro under appropriate condition) to, e.g., corneal epithelial cells (CECs).

“Limbal stem or progenitor cells” or “LSCs” include stem cells obtained from, e.g., the limbus, a region between cornea and conjunctiva of an eye. LSCs can proliferate and differentiate to give rise to corneal epithelial cells (CECs). In particular, LSCs are thought to reside in LSC niche within the limbus. LSCs may be isolated from limbus region comprising corneal limbus of an eye, margin between cornea and conjunctiva, border of cornea and sclera, corneoscleral limbus, a crypt region of the basal layer of limbal epithelium, a region comprising interpalisade rete ridge, or a region comprising Palisades of Vogt.

As defined herein “isolated” refers to material removed from its original environment and is thus altered “by the hand of man” from its natural state.

“Isolated limbal stem or progenitor cells” include LSCs isolated from an individual and placed in ex vivo or in vitro culture. Typically, isolated LSCs in a tissue biopsy may be dissociated to obtain single cells.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “totipotent cells” shall have the following meaning. In mammals, totipotent cells have the potential to become any cell type in the adult body; any cell type(s) of the extraembryonic membranes (e.g., placenta). Totipotent cells include the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploidy.

As used herein, the term “multipotent stem cells” are true stem cells but can only differentiate into a limited number of types. For example, the bone marrow contains multipotent stem cells that give rise to all the cells of the blood but may not be able to differentiate into other cells types.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated.

As used herein, the term “feeder cells” is intended to mean additional cells playing a role as an aid, which are used to adjust culture conditions, for example, for target pluripotent stem cells to be proliferated or differentiated. For example, feeder cells, particularly animal feeder cells such as mouse-derived primary cultured fibroblasts, are responsible for providing a scaffold for cell adhesion and supplying growth factors required for stem cells. Accordingly, by “feeder free” or “free” of feeder cells is meant that no feeder cells are used in the preparation, growth, culturing, expansion, storage or formulation of the certain composition or process.

By the term “expanded,” in reference to cell compositions, means that the cell population constitutes a significantly higher concentration of cells than is obtained using previous methods. For example, an “expanded” population has at least a 2 fold, and up to a 10 fold, improvement in cell numbers per gram of tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (e.g., diluted 1:5) and placed into a new tissue culture vessel to allow for their continued growth and viability. For example, LSCs isolated from the limbus are referred to as primary cells. Such cells are expanded in culture by being grown in the growth medium described herein. When such primary cells are subcultured, each round of subculturing is referred to as a passage. As used herein, “primary culture” means the freshly isolated cell population from a subject.

As used herein, the term “differentiation” means the process by which cells become progressively more specialized. When used herein to describe pluripotent stem cells, the term “differentiation” is intended to mean a change that causes the pluripotent stem cells to lose their differentiation pluripotency (i.e., potential ability to differentiate into all tissues) and to have characters as cells constituting a specific tissue.

The term “physiological level” as used herein means the level that a substance in a living system is found and that is relevant to the proper functioning of a biochemical and/or biological process.

As used herein, the term “pooled” means a plurality of compositions that have been combined to create a new composition having more constant or consistent characteristics as compared to the non-pooled compositions.

The term “therapeutically effective amount” means that amount of a therapeutic agent necessary to achieve a desired physiological effect (for example, repair or promote corneal healing).

The term “lysate” as used herein refers to the composition obtained when cells, for example, LSCs are lysed and optionally the cellular debris (e.g., cellular membranes) is removed. This may be achieved by mechanical means, by freezing and thawing, by sonication, by use of detergents, such as EDTA, or by enzymatic digestion using, for example, trypsin, chymotrypsin, collagenases, elastase, hyaluronidase, dispase, proteases, and nucleases, as well as commercial products such as Stem Pro Accutase. In some instances, it may be desirable to lyse the cells and retain the cellular membrane portion and discard the remaining portion of the lysed cells.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

The term “transplantation” as used herein refers to the administration of a composition comprising cells, including a cell suspension or cells incorporated into a matrix or tissue, that are either in an undifferentiated, partially differentiated, or fully differentiated form, into a human or other animal.

As used herein, the term “adjunctive” means jointly, together with, in addition to, in conjunction with, and the like.

As used herein, the term “co-administer” can include simultaneous or sequential administration of two or more agents.

The terms “subject” and “individual” are used interchangeably. As used herein, both terms mean any animal, such as a mammal, including a human and/or non-human. The terms patient, subject, and individual are used interchangeably. None of the terms are to be interpreted as requiring the supervision of a medical professional (e.g., a doctor, nurse, physician's assistant, orderly, hospice worker).

The terms “treat,” “treating” or “treatment,” as used herein, include alleviating, abating and/or ameliorating a disease and/or condition symptoms, preventing additional symptoms, ameliorating and/or preventing the underlying metabolic causes of symptoms, inhibiting the disease and/or condition, e.g., arresting the development of the disease and/or condition, relieving the disease and/or condition, causing regression of the disease and/or condition, relieving a condition caused by the disease and/or condition, and/or stopping the symptoms of the disease and/or condition either prophylactically or therapeutically.

The term “ophthalmically acceptable” with respect to a formulation, composition or ingredient as used herein means having no persistent effect that is substantially detrimental to the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, compositions and ingredients are those that cause no substantial detrimental effect, even of a transient nature.

As used herein, the term “matrix” refers to any substance to which the limbal stem cells and/or progenitors thereof can adhere and which therefore can substitute the cell attachment function of feeder cells, or supports the adherence thereof, such as an attachment factor. Particularly suitable for use with the present invention are extracellular matrix components derived from basement membrane or extracellular matrix components that form part of adhesion molecule receptor-ligand couplings. Non-limiting examples of suitable matrices which can be used by the method of this aspect of the present invention include mammalian amniotic membrane (e.g., human amniotic membrane), collagen (e.g., collagen IV), fibrinogen, perlecan, laminin, fibronectin, proteoglycan, procollagens, hyaluronic acid, entactin, heparan sulfate, tenascin, poly-L-lysine, gelatin, poly-L-ornithine, and the like, or any combinations thereof. Alternatively, the extracellular matrix is commercially available. Examples of commercially available extracellular matrices are extracellular matrix proteins (Fischer or Life Tech), fibrinogen and thrombin sheet (Reliance Life), and Matrigel™ (BD Biosciences) and their equivalents. In cases where complete animal-free culturing conditions are desired, the matrix is derived from a human source or synthesized using recombinant techniques. Such matrices include, for example, human amniotic membrane, human-derived fibronectin, recombinant fibronectin matrix which can be obtained from Sigma, St. Louis, Mo., USA or can be produced using known recombinant DNA technology (see, for example, U.S. Pat. No. 6,152,142, and Tseng et al., (1997) Am. J. Ophthalmol. 124:765-774, each incorporated herein by reference).

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes 1-III; Celis, ed., 1994, “Cell Biology: A Laboratory Handbook” Volumes 1-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

As used herein, the term “about” when used before a numerical designation, e.g., temperature, time, amount, concentration, and such other, including a range, indicates approximations which may vary by (+) or (−) 10%, 5% or 1%.

As used herein, the term “substantially free” includes being free of a given substance or cell type or nearly free of that substance or cell type, e.g. having less than about 1% of the given substance or cell type.

It must be noted that as used herein and in the appended claims, the singular forms “a,” “and” and “the” include plural references unless the context clearly dictates otherwise.

As used herein, the terms “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the present disclosure. “Consisting of” shall mean excluding more than trace elements of other ingredients and substantial method steps. Embodiments defined by each of these transition terms are within the scope of the present disclosure.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

### COMPOSITIONS OF THE INVENTION

The invention provides an isolated limbal stem or progenitor cell (LSC) population or LSC-like population comprising a chemically synthesized, recombinant or isolated nucleic acid encoding PAX6 integrated into a chromosome, or alternatively, not integrated remaining as an extrachromosomal genetic material, wherein the isolated LSC population is substantially free of non-LSC cells or wherein the LSC-like population is substantially free of non-LSC-like cells, or wherein the isolated LSC or LSC-like population is substantially free of non-LSC and non-LSC-like cells. The LSC population or LSC-like population may be from a mammal such as a human. The LSC population or LSC-like population may be genetically modified. Further, the LSC population or LSC-like population may remain or may be maintained as LSC or LSC-like cell fate.

In one embodiment of the invention, the chemically synthesized, recombinant or isolated nucleic acid can express PAX6 or a fragment thereof. The PAX6 or a fragment thereof may maintain LSC or LSC-like state or can direct a stem cell or progenitor cell to a LSC or LSC-like state. Further, the LSC or LSC-like state may restrict a cell population to a differentiation pathway resulting in corneal epithelial cells (CECs). In another embodiment, the PAX6 or a fragment thereof maintains LSC or LSC-like state or directs a stem cell or progenitor cell to a LSC or LSC-like state, and further the LSC or LSC-like state restricts a cell population to a differentiation pathway resulting in corneal epithelial cells.

In a further embodiment of the invention, 90-95% of the LSC population or LSC-like population expresses p63, PAX6, K19 and Ki67. In another embodiment, less than 5% of the LSC population expresses K5 and K14. In yet another embodiment, greater than 95% of the LSC population expresses WNT7A and FZD5. In yet a further embodiment, less than 5% of the LSC-like population expresses WNT7A.

In another embodiment of the invention, the corneal epithelial cells express PAX6 and corneal epithelial markers, K3 and K12.

In one embodiment, Isolated LSCs express a set of markers comprising WNT7A, FZD5, PAX6, p63, keratin 5 (K5), keratin 14 (K14), keratin 19 (K19), and Ki67. In another embodiment, 90-95% of the LSCs express p63, PAX6. K19 and Ki67. In yet another embodiment, greater than 95% of the LSCs express WNT7A and FZD5. In another embodiment, less than 5% of the LSCs express K5 and K14. In yet a further embodiment, 90-95% of the LSCs express p63, PAX6, K19 and Ki67, greater than 95% of the LSCs express WNT7A and FZD5, and less than 5% of the LSCs express K5 and K14.

For example, Limbal stem or progenitor cell-like cells or LSC-like cells are non-LSC stem cells, e.g., skin epithelial stem cells (SESCs), which upon overexpression of PAX6 to a sufficient amount switches or adopts a “LSC-like” state. In one embodiment, a stem cell switched to a “LSC-like” state has induced K19 expression coincident with expression of both p63 and PAX6 in the nucleus. In another embodiment, when a “LSC-like” cell is placed in three-dimensional culture (embedded in reduced growth factor Matrigel) in the presence of LSC differentiation medium, the “LSC-like” cells differentiate to “CEC-like” cells with increased corneal K3 and K12 expression and concomitant decreased skin K1 and K10 expression. For example, corneal K3 expression may be about 9.4-fold higher in CEC-like cells produced by 3D differentiation of PAX6-overexpressed SESCs (which convert to a LSC lineage rather than remain as committed SESCs) than similarly treated SESCs but not overexpressing PAX6.

The invention further provides a defined cell population comprising a plurality of the cells of LSC population or LSC-like population. The defined cell population may be homogenous or heterogeneous. The defined cell population may be clonal or derived from a single cell. The invention further provides a progeny cell of the LSC population or LSC-like population, committed to develop into a corneal epithelial cell. Additionally, the invention also provides tissue comprised of the cells of the LSC population or LSC-like population.

The invention further provides pharmaceutical compositions comprising the LSC population or LSC-like population and a suitable carrier. In one embodiment of the invention, the LSC population or LSC-like population may be be cultured for at least 17 passages without differentiating to CECs. Additionally, in another embodiment of the invention, the percentage of cells expressing PAX6 and p63 at passage 3 is the same as the percentage at passage 17. In a further embodiment of the invention, the percentage of cells expressing K19 and Ki67 at passage 3 is slightly greater than the percentage at passage 17 or later. In yet a further embodiment of the invention the LSC population or LSC-like population of the invention comprises cells that may be stably propagated for 40-60 generations without differentiating to CECs. Further, in one embodiment of the invention, the LSC population or LSC-like population may differentiate into a corneal epithelial cell population.

Additionally, the invention also provides tissue comprised of the cells the LSC population or LSC-like population of the invention. Further, the invention additional provides methods of forming tissue in a subject comprising introducing the progeny cell of the the LSC population or LSC-like population of the invention into or onto a subject in a sufficient amount to form corneal epithelial cells in said subject.

The invention additionally provides an isolated skin epithelial stem cell (SESC) population or SESC-like population comprising a chemically synthesized, recombinant or isolated nucleic acid encoding PAX6 integrated into a chromosome, or alternatively, not integrated, remaining as an extrachromosomal genetic material, wherein the isolated SESC population is substantially free of non-SESC cells. Further, the SESC-like population may be substantially free of non-SESC-like cells, or the isolated SESC or SESC-like population may be substantially free of non-SESC and non-SESC-like cells, or further still the isolated SESC or SESC-like population may be substantially free of non-SESC, non-SESC-like, non-LSC and non-LSC-like cells. Additionally, in one embodiment of the invention, the chemically synthesized, recombinant or isolated nucleic acid can express PAX6 or a fragment thereof, which can maintain LSC or LSC-like state or can direct a stem cell or progenitor cell to a LSC or LSC-like state, and which, in turn, may restrict the cell population to a differentiation pathway resulting in corneal epithelial cells. In another embodiment of the invention, the chemically synthesized, recombinant or isolated nucleic acid expresses PAX6 or a fragment thereof, which directs SESC or SESC-like cell to a LSC or LSC-like state, which, in turn, may restrict the cell population to a differentiation pathway resulting in corneal epithelial cells. The SESC population or SESC-like population may be from a mammal such as a human. The SESC population or SESC-like population may be genetically modified. Further, the SESC population or SESC-like population may switch from a SESC or SESC-like cell fate to a LSC or LSC-like cell fate.

In one embodiment, about 90-95% of the cell population expresses p63, K5 and Ki67 while remaining in a SESC or SESC-like cell fate. In another embodiment, about K3 or K12 expression is not detected in cells remaining in a SESC or SESC-like cell fate. In yet a further embodiment, WNT7A is expressed in cells remaining in a SESC or SESC-like cell fate at about 4-5 fold lower level than the level in LSC cells. In yet another embodiment, PAX6 is not expressed or expressed in cells remaining in a SESC or SESC-like cell fate at a level less than about one eighth of the level in LSC cells. In an additional embodiment, WNT7A is expressed in more than 70% of cells remaining in a SESC-like fate. In yet an additional embodiment, 90-95% of cells in the population that switched to a LSC or LSC-like cell fate expresses p63, PAX6, K19 and Ki67. Additionally, I an embodiment of the invention, the skin epidermal cells express skin epidermal differentiation markers, K1 and K10.

Additionally, the invention further provides pharmaceutical compositions comprising the SESC population or SESC-like population and a suitable carrier. In one embodiment of the invention, the SESC population or SESC-like population of the invention may be cultured for at least 17 passages without differentiating to skin epidermal cells or corneal epithelial cells. In one embodiment, the SESC population or SESC-like population may comprise cells which can be stably propagated for 40-60 generations without differentiating to skin epidermal cells or corneal epithelial cells. In a further embodiment, the SESC population or SESC-like population may comprise SESC or SESC-like cells that have switched cell fate to a LSC or LSC-like cell fate. Further, in one embodiment, the SESC population or SESC-like population has adopted a LSC or LSC-like cell fate and the SESC or SESC-like cell fate is absent. The SESC population or SESC-like population may differentiate into corneal epithelial cells. In one embodiment, the SESC population or SESC-like population may differentiate into corneal epithelial cells, which are substantially free of skin epidermal cells.

The invention further provides a defined cell population comprising a plurality of the cells of SESC population or SESC-like population. The defined cell population may be homogenous or heterogeneous. The defined cell population may be clonal or derived from a single cell. The invention further provides a progeny cell of the SESC population or SESC-like population, committed to develop into a corneal epithelial cell.

Additionally, the invention also provides tissue comprised of the cells of the SESC population or SESC-like population of the invention. Further, the invention additional provides methods of forming tissue in a subject comprising introducing the progeny cell of the SESC population or SESC-like population of the invention into or onto a subject in a sufficient amount to form corneal epithelial cells in said subject.

### METHODS OF THE INVENTION

In one embodiment of the invention, the present disclosure relates to the culture of mammalian limbal stem cells (LSCs). The limbal stem cells are derived from corneoscleral or corneal limbus tissue from a human donor. In particular, the present disclosure is a system with self-regenerating limbal stem cells, and can comprise a large population of LSCs, for example at least about 70%, at least about 80%, or at least about 90% limbal stem cells. A typical procedure for isolating corneal limbal tissue is to surgically remove a small biopsy consisting of 0.8-3 mm² of limbal tissue from the superior or temporal quadrant of the corneal surface of the donor's eye. Procedures for obtaining such biopsies from the corneal limbus, for example by lamellar keratectomy, are known to those of skill in the art. The donor of the limbal tissue biopsy used to generate the limbal stem cells may also be the recipient of the tissue system transplant, implant, or graft (i.e., autologous tissue system). Alternatively, when the donor of the limbal tissue biopsy is not the recipient, the donor is in an example a bio-compatible donor, for example a close relative of the recipient of the transplant or graft, or may also be from a bio-compatible (e.g., histocompatible) cadaver (i.e., allogeneic tissue system). It is generally desirable that transplanted cells or tissues be genetically compatible or identical to the recipient of the transplant in order to avoid problems with tissue rejection.

The LSCs of the present disclosure are undifferentiated or substantially undifferentiated cells that have the potential to differentiate into corneal epithelial cells. Morphological characteristics of undifferentiated cells are well known to those of skill in the art. Those of skill in this technology understand that cells useful in embodiments of the invention such as limbal stem cells of the corneal epithelium can be characterized by a number of complementary factors such as the in vivo site from which they are obtained, and/or their morphology or size (e.g. average diameter), as well as the presence, absence and/or expression levels of biomarkers such as the ATP-binding cassette subfamily G member 2 (ABCG2), transcription factor p63, Bmi-1, Notch-1, stage-specific embryonic antigen-4 (SSEA4), stage-specific embryonic antigen-3 (SSEA3), N-cadherin. CD73, CD105, CD54, CD117, Oct-4, Ki67, Nanog, Rex 1, Sox2, Tra-1-60, Tra-1-81, Stem Cell Factor, and cytokeratins (K) such as K1, K3, K5, K10, K12, K14 or K15, K19 and Desmoglein-3 (see, e.g. Nakatsu et al., Investigative Ophthalmology & Visual Science 2011; 52:4734-4741; Truong et al., Invest Ophthalmol Vis Sci. 2011; 52:6315-6320; Dua et al., Surv Ophthalmol. 2000 March-April; 44(5):415-25; Watson et al., Curr Eye Res. 2013 Apr. 10; Meyer-Blazejewska et al., Invest Ophthalmol Vis Sci. 2010 February; 51(2):765-74; and Rama et al., N Engl J Med 2010; 363: 147-55; Thomson et al., (Science 282:1145-1147, 1998), Reubinoff et al. (Nature Biotech. 18:399-403, 2000)). In illustrative embodiments of the invention, the human limbal stem cells exhibit an expression profile characterized by examining the expression of one or more of ATP-binding cassette subfamily G member 2 (ABCG2), -transcription factor p63a, stage-specific embryonic antigen-4 (SSEA4), N-cadherin, and cytokeratins (K) such as K1, K3, K5, K10, K12, K14 or K15. In embodiments of the invention, other characteristics of the human limbal stem cells or feeder cells are also identified or characterized, for example cellular size or morphology.

For example, once a biopsy is removed from a donor, it must be cared for in a manner such that a sufficient portion of the limbal tissue biopsy remains viable so as to permit isolation of limbal stem cells. In one embodiment, the limbal tissue biopsy is transported or stored in a medium which supports the viability of the biopsy. An example of medium for storing or transporting the biopsy can comprise Dulbecco's Modified Eagles Medium (DMEM) and Ham's F-12 (ratio 1:1), DMSO (0.1-0.5%), recombinant human epidermal growth factor (rhEGF; 0.5-2 ng/ml), insulin (0.5-5 μg/ml), transferrin (0.5-5 μg/ml), sodium selenite (0.5-5 μg/ml), hydrocortisone (0.1-0.5 μg/ml), cholera toxin A (0.01-0.1 μmol/l), gentamycin (10-50 μg/ml), and amphotericin B (0.5-1.25 μg/ml). Alternatively, the medium may be substituted with functionally equivalent components or with different antibiotics. The medium can further be supplemented with human cord blood serum (3-5%). The limbal cell biopsies can be placed in culture within 48 hours of surgical removal from the donor.

Limbal stem cells may be purified directly from tissue biopsy prior or subsequent to transportation. The limbal tissue biopsy may either be cultured as an intact explant, or may be dissociated into a single (or reduced) cell suspension prior to being cultured. For example, the tissue may be purified for certain populations with increased biological activity. Purification may be performed using means known in the art, or may be achieved by positive selection for LSC markers as described herein. In one embodiment of the invention, limbal stem cells are mechanically degraded in a sterile manner and treated with enzymes to allow dissociation of the cells from the collected tissue. Such enzymes include, but not restricted to trypsin, chymotrypsin, collagenases, elastase hylauronidase and/or commercial products such as Stem Pro Accutase (Fischer). Suspension of limbal stem cells are subsequently washed, assessed for viability, and may either be used directly for the practice of the invention or cultured for expansion. In some situations it will be desirable to expand cells before use for generation by conditioned media. Expansion can be performed by culture ex vivo with specific factors as described herein.

After limbal tissue is biopsied from a donor, it is placed in culture with culture media, and in one embodiment with an appropriate support matrix, such as an extracellular matrix or biocoated surface, for example extracellular matrix carrier or biocoated petri dishes. In one embodiment, the presence of a support matrix facilitates the binding of the limbal stem cells in the biopsy to the tissue culture plate or vessel, thereby facilitating the growth of the limbal stem cells. The explant can be cut into small pieces before being placed in culture.

Human amniotic membrane may be prepared to enhance the growth of limbal stem cells by removing endogenous amniotic epithelial cells by freeze-thawing, enzymatic digestion, and mechanical scraping, followed by the treatment of the surface with growth factors, extracellular matrix compounds, and/or adherence-enhancing molecules. In one embodiment, the amniotic membrane, with the basement membrane or stromal side up, is affixed smoothly onto a culture plate for culturing LSCs.

Any medium capable of supporting LSCs in vitro may be used to culture the LSC's. Media formulations that can support the growth of LSCs include, but are not limited to, Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's salt base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non essential amino acids), alpha modified Minimal Essential Medium (aMEM), and Roswell Park Memorial Institute Media 1640 (RPMI Media 1640), EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media, and the like; among numerous others, including medium 199, CMRL 1415, CMRL 1969, CMRL 1066, NCTC 135, MB 75261, MAB 8713. DM 145, Williams' G, Neuman & Tytell, Higuchi, MCDB 301, MCDB 202, MCDB 501, MCDB 401, MCDB 411, MDBC 153. A preferred medium for use in the present invention is DMEM. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62-72, edited by William B. Jakoby and Ira H. Pastan, published by Academic Press, Inc.

Additional non-limiting examples of media useful in the methods of the invention can contain fetal serum of bovine, calf serum or serum of other species at a concentration of at least 1% to about 30%, at least about 5% to 15%, e.g., about 10%. In certain embodiments, it may be preferably to use human serum.

In a preferred embodiments, the medium used in the invention is a feeder-free, serum-free and xeno-free medium. For example, the media used to prepare the tissue system, including the medium used to transport the limbal tissue biopsies, the medium used to culture the biopsies, the enriched medium used to culture the limbal stem cells, and the medium used to transport the tissue system, do not contain any sera or other factors of animal origin. This will help minimize any risk of contamination of the tissue system with xenogenic components, thereby making the tissue systems safe for human administration. In a most preferred embodiment, the feeder-free, serum-free and xeno-free medium is a chemically defined medium in which all components in the medium are chemically defined and do not contain undefined animal-derived or human-derived products.

For general techniques relating to cell culture and culturing stem cells, which can be applied to culturing LSCs, the practitioner can refer to standard textbooks and reviews, for example: E. J. Robertson, “Teratocarcinomas and embryonic stem cells: A practical approach” ed., IRL Press Ltd. 1987; Hu and Aunins (1997), Curr. Opin. Biotechnol. 8:148-153; Kitano (1991), Biotechnology 17:73-106; Spier (1991), Curr. Opin. Biotechnol. 2:375-79; Birch and Arathoon (1990), Bioprocess Technol. 10:251-70; Xu et al. (2001), Nat. Biotechnol. 19(10):971-4; and Lebkowski et al. (2001) Cancer J. 7 Suppl. 2:S83-93, each incorporated herein by reference.

A further embodiment of a method according to the invention comprises a culture medium comprising a ROCK (Rho-associated protein kinase) inhibitor. The addition of a ROCK inhibitor was found to prevent anoikis, especially when culturing stem cells. The ROCK inhibitor are known in the art and in one example, selected from R)-(+)-trans-4-(1-aminoethyl)-N-(4-Pyridyl)cyclohexanecarboxamide dihydrochloride monohydrate (Y-27632; Sigma-Aldrich), 5-(1,4-diazepan-1-ylsulfonyl) isoquinoline (fasudil or HA 1077; Cayman Chemical), H-1152, H-1152P, (S)-(+)-2-Methyl-1-[(4-methyl-5-isoquinolinyl)sulfonyl]homopiperazine, 2HCl, ROCK Inhibitor, Dimethylfasudil (diMF, H-1152P), N-(4-Pyridyl)-N′-(2,4,6-trichlorophenyl)urea, Y-39983, Wf-536, SNJ-1656, and (S)-+)-2-methyl-1-[(4-methyl-5-isoquinolinyl)sulfonyl]-hexahydro-1H-1,4-diazepine dihydrochloride (H-1152; Tocris Bioscience), and its derivatives and analogs. Companies currently developing Rho-kinase inhibitors include Senju Pharmaceuticals (Osaka, Japan), Novartis (Basel, Switzerland), Kowa Pharmaceutical (Nagoya, Japan), Santen, working with Ube Industries (Tokyo, Japan), and Inspire Pharmaceuticals (Durham, N.C.). —See more in a review on Rho-kinase inhibitors by Lama Al-Aswad, published Mar. 20, 2009 in an online newsletter associated with an online journal, Review of Opthalmology Online at: http://www.revophth.com/content/d/glaucoma_management/d/1222/p/23008/c/22947/#sthash.H1 6F1ABU.dpuf. Additional ROCK inhibitors include imidazole-containing benzodiazepines and analogs (see, e.g., WO 97/30992). Others include those described in International Application Publication Nos.: WO 01/56988; WO 02/100833; WO 03/059913; WO 02/076976; WO 04/029045; WO 03/064397; WO 04/039796; WO 05/003101; WO 02/085909; WO 03/082808; WO 03/080610; WO 04/112719; WO 03/062225; and WO 03/062227, for example. In some of these cases, motifs in the inhibitors include an indazole core; a 2-aminopyridine/pyrimidine core; a 9-deazaguanine derivative; benzamide—comprising; aminofurazan—comprising; and/or a combination thereof.

Rock inhibitors also include negative regulators of ROCK activation such as small GTP-binding proteins (e.g., Gem, RhoE, and Rad), which can attenuate ROCK activity. In specific embodiments of the disclosure, ROCK1 is targeted instead of ROCK2, for example, WO 03/080610 relates to imidazopyridine derivatives as kinase inhibitors, such as ROCK inhibitors, and methods for inhibiting the effects of ROCK1 and/or ROCK2. The disclosures of the applications cited above are incorporated herein by reference. The Rho inhibitor can also act downstream by interaction with ROCK (Rho-activated kinase) leading to an inhibition of Rho. Such inhibitors are described in U.S. Pat. No. 6,642,263 (the disclosures of which are incorporated by reference herein in their entirety). Other Rho inhibitors that may be used are described in U.S. Pat. Nos. 6,642,263, and 6,451,825. Such inhibitors can be identified using conventional cell screening assays, e.g., described in U.S. Pat. No. 6,620,591 (all of which are herein incorporated by reference in their entirety).

Culture medium can also include growth factors, cytokines, and hormones necessary for culturing LSCs at appropriate concentrations in the medium. Media useful in the methods of the invention may also contain one or more compounds of interest, including but not limited to antibiotics, anti-inflammatory, antiviral, mitogenic or differentiation compounds useful for the culturing of LSCs. The cells may be grown in one non-limiting embodiment, at temperatures between about 27° C. to 40° C., in another non-limiting embodiment at about 31° C. to 37° C., and in another non-limiting embodiment in a humidified incubator. The carbon dioxide content may be maintained between about 2% to 10% and the oxygen content may be maintained between about 1% and 22%; however, the invention should in no way be construed to be limited to any one method of isolating and culturing LSCs. Rather, any method of isolating and culturing LSCs should be construed to be included in the present invention.

The media may also be supplemented with growth factors. As used herein, the term “growth factor” refers to proteins that bind to receptors on the cell surface with the primary result of activating cellular proliferation and/or differentiation. The growth factors used for culturing limbal tissue are, for example, selected from epidermal growth factor (EGF), basic fibroblast growth factor (bFGF), leukemia inhibitory factor (LIF), nerve growth factor (NGF), insulin growth factor (IGF), TGF-beta, hepatocyte growth factor, keratinocyte growth factor, insulin, sodium selenite, human transferrin, or human leukemia inhibitory factor (hLIF), bovine pituitary extract, and the like, as well as combinations thereof. However, any suitable culture media known to those of skill in the art may be used. In certain embodiments, the limbal cells are treated with cytokines or other growth factors that cause the LSCs to preferably proliferate in the culture. Other factors used for culturing limbal cells can be selected from DMSO and hydrocortisone, glucose, L-glutamine, folic acid, sodium bicarbonate, adenine, CaCl2, progesterone, ethanolamine, triiodothyronine, phosphorylethanolamine, and the like.

In certain embodiments, the antibiotic is a macrolide (e.g., tobramycin (TOBI®)), a cephalosporin (e.g., cephalexin (KEFLEX®)), cephradine (VELOSEF®)), cefuroxime (CEFTIN®, cefprozil (CEFZIL®), cefaclor (CECLOR®), cefixime (SUPRAX® or cefadroxil (DURICEF®), a clarithromycin (e.g., clarithromycin (Biaxin)), an erythromycin (e.g., erythromycin (EMYCIN®)), a penicillin (e.g., penicillin V (V-CILLINK® or PEN VEEK®)) or a quinolone (e.g., ofloxacin (FLOXIN®), ciprofloxacin (CIPRO®) ornorfloxacin (NOROXIN®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, methotrexate, colchicine, allopurinol, probenecid, sulfinpyrazone and benzbromarone.

Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons.

In one embodiment, the isolated population of LSC's is cultured in medium that will allow the cells to expand without substantially differentiating, for example in culture medium enriched with conditioned medium obtained from inactivated human embryonic fibroblast cells, culture medium enriched with human leukemia inhibitory factor, or culture medium supplemented with one or more soluble factors selected from the group consisting of dimethyl sulphoxide, recombinant human epidermal growth factor, insulin, sodium selenite, transferrin, progesterone, putrescine, selenite salt, hydrocortisone, and basic fibroblast growth factor. Alternatively, the LSCs are cultured in medium that will allow the cells to differentiate into, for example, corneal epithelial cells, using, for example, specific growth factors or media, e.g. corneal epithelium culture media CnT-30 (CELLnTEC, Zen-Bio) or chemically defined xeno-free culture medium, RegES (Regea 06/015, Regea 07/046, and Regea 08/013; Rajala et al., 2010, PLOS One 5(4):e10246).

An exemplary method of culturing the limbal tissue biopsies is to subject the explant to dry incubation for several minutes, either before or after placing the explant on an extracellular matrix or biocoated tissue culture plate. A small amount of culture medium is then added to the explant so that it sticks to the extracellular matrix or biocoated tissue culture surface. After several hours to a day, additional media is gently added and the explant is incubated for several days at 37° C. in a CO₂ incubator, changing the media every alternate day. Preferably, in such an example, the pieces of the original limbal tissue biopsy are removed from the culture after stem cells begin proliferating in the culture.

In other embodiments, prior to expansion, the limbal tissue biopsy may be used to generate a single cell suspension, which is subsequently cultured to generate the tissue system disclosed herein. For example, the limbal tissue biopsy is washed and then enzymatically treated, for example with trypsin-EDTA (e.g., about 0.25% for 20-30 minutes) or dispase (e.g., overnight at 4° C.), to generate a single cell suspension which includes LSCs. Enzymatic treatment allows for separation of the epithelium; therefore, stroma or mesenchymal cells may be reduced or absent in the single cell suspension.

After the limbal tissue is cultured for several days, for example until the cells become confluent, the LSCs can be isolated from the culture. Preferably, in this example, the limbal tissue culture is allowed to grow until it is at least about 50%, 60%, 70%, 80%, 90%, or 95% confluent. In one embodiment, the limbal cells are first dissociated from the extracellular matrix or biocoated tissue culture plate, for example, through enzymatic digestion, for example using trypsin-EDTA or dispase solutions. The LSCs can also be isolated from the other cells in the culture using a variety of methods known to those of skill in the art such as immunolabeling and fluorescence sorting, for example solid phase adsorption, fluorescence-activated cell sorting (FACS), magnetic-affinity cell sorting (MACS), and the like. In certain embodiments, the LSCs are isolated through sorting, for example immunofluorescence sorting of certain cell-surface markers. Two preferred methods of sorting well known to those of skill in the art are MACS and FACS.

Sorting techniques may involve the use of appropriate stem cell markers to separate LSCs from other cells in the culture. LSCs may be identified by one or more specific surface markers using one or more labeled-antibody or factor that interact specifically with the marker and sorted on the basis of the presence of a label such as a fluorescent label in the case of FACS or paramagnetic material in the case of MACS. Appropriate stem cell specific surface markers that may be used to isolate LSCs from cultured limbal cells include but are not limited to ABCG2, transcription factor p63, SSEA4, SSEA3, N-cadherin, CD73, CD105, CD54, CD117, Oct-4, Nanog, TDGF, UTX-1, FGF-4, Rex 1, Sox2, Tra-1-60, Tra-1-81, Stem Cell Factor, and K1, K3, K10, K12, K14 or K15, K19. By this means, enriched populations of cell-surface marker positive for LSCs are obtained from the mixed population of cells cultured from the limbal tissue biopsy. Alternatively, the cells can be sorted to remove undesirable cells by selecting for cell-surface markers not found on LSCs. In the case of LSCs isolated from limbal tissue, LSCs are negative for the following cell-surface markers: CD34. CD45. CD14, CD133, CD106, CD11c, CD123, and HLA-DR.

The enriched limbal cell cultures obtained by sorting have at least about 10%. 20%, 30%, 40%, 50%, 60%, 70%, 80%, 90%, 95%, 98%, or 99% LSCs. In alternative embodiments, mixed cell cultures containing limbal cells are screened for the presence of LSCs by screening for expression of certain gene markers. In the case of mixed limbal cell cultures, populations of LSCs can be identified by the expression of gene markers such as ABCG2, transcription factor p63, SSEA4, SSEA3, N-cadherin, CD73, CD105, CD54, CD117, Oct-4, Nanog, TDGF, UTX-1, FGF-4, Rex 1, Sox2, Tra-1-60, Tra-1-81, Stem Cell Factor, and K1, K3, K10, K12, K14 or K15, K19, as well as other gene marker of undifferentiated cells, or combinations thereof.

After the population of limbal cells enriched for LSCs is isolated using one of the above methods, the isolated cells are preferably cultured under conditions and in a medium that supports the growth of LSCs and the development of a tissue system for transplanting, implanting, or grafting onto a damaged or diseased eye. Preferably the tissue system cultured under these conditions will comprise at least about 30%, 40%, 50%, 60%, 70%, 80%, 90%, or 95% LSCs. In an embodiment, the isolated LSCs are cultured on a tissue base in the presence of an enriched medium for developing the tissue system with LSCs. Different factors can be added at varying stages of growth or expansion. For example, ROCK inhibitors can be added after several passages of expansion. The tissue base can have characteristics which approximate the natural ocular surface, for example characteristics such as being clear, thin, elastic, biocompatible, non-vascular, and non-antigenic, and can also support the growth of LSCs, as well as normal differentiation after transplant, implant, or graft.

The limbal cells comprising LSCs are cultured or passaged in an appropriate medium to allow the LSCs to remain in a substantially undifferentiated state. Although colonies of LSCs within the population may be adjacent to neighboring cells that are differentiated, the culture of LSCs will nevertheless remain substantially undifferentiated when the population is cultured or passaged under appropriate conditions, and individual LSCs constitute a substantial proportion of the cell population. Undifferentiated stem cell cultures that are substantially undifferentiated contain at least about 20% undifferentiated LSCs, and may contain at least about 40%, 60%, 80%, or 90% LSCs. For example, LSCs in culture must be kept at an appropriate cell density at about 10⁴/cm², and subcultured, while frequently exchanging the culture medium to prevent them from differentiating. In long term culture, when the cells are passaged at about 70-90% confluence, they may be dispersed into small clusters or into single-cell suspensions. Typically, a single cell suspension of cells is achieved and then seeded onto another tissue culture grade plastic dish to achieve about 15-20% confluence after passage.

The cultures can be serially passaged for at least 10, 20, 40, 60, 80, 100 or more passages, without LSCs substantially differentiating. The limbal cell cultures comprising LSCs can be cryopreserved for further use at various time points without loss of differentiation potential, preferably, in such case, in freezing medium that comprises culture medium with about 10-90% heat inactivated serum collected from human cord blood and about 5-10% DMSO. Alternatively, it is anticipated that the freezing medium may be a chemically defined which is serum-free, xeno-free and feeder-free. The limbal cell cultures can comprise LSCs, preserved after every passage, for example by cryopreserving, so that additional or multiple tissue systems can be generated from a single limbal tissue biopsy. These cryopreserved cultures will also serve as a pool of undifferentiated, self-regenerating, and viable limbal stem cells for future use at any given point in time. For example, these cryopreserved cultures may be used to generate additional tissue systems for autologous use in the event of a failure of the tissue system in the recipient due to immunosuppression, complications from prior surgery, infection, and the like. These cryopreserved cultures may also be used to generate additional tissue systems for biocompatible patients. The availability of these preserved cultures will also obviate the need to remove additional limbal tissue from a donor in the event the tissue system fails, thereby preventing the risk of exhausting a source of autologous limbal stem cells in the future.

After the tissue system disclosed herein is generated, it may be transported to the recipient's location for transplant, implant, or graft. The means used to transport the tissue system can maintain the viability of the tissue system sufficiently that it is still useful as a transplant, implant, or graft after transport. The tissue system is transported in a receptacle that contains transportation medium, which can be the enriched medium used to culture the tissue system comprising LSCs or an alternate medium sufficient to buffer the tissue system during transport without growth factors. Alternatively, the recipient may be brought to a facility holding the tissue system disclosed herein, obviating a need for a transportation medium.

The tissue system comprising LSCs disclosed herein can be utilized for therapeutic applications, for example as transplants, implants, or grafts for subjects with limbal stem cell deficiencies in one or both eyes. The tissue system of the present disclosure can be used to treat any subject in need of treatment, including but not limited to humans, primates, and domestic, farm, pet, or sports animals, such as dogs, horses, cats, sheep, pigs, cattle, rats, mice, and the like. As used herein, the terms “therapeutic”, “therapeutically”, “to treat”, “treatment”, or “therapy” refer to both therapeutic treatment and prophylactic or preventative measures. Therapeutic treatment includes but is not limited to reducing or eliminating the symptoms of a particular disease, condition, injury or disorder, or slowing or attenuating the progression of, or curing an existing disease or disorder.

Subjects in need of such therapy will be treated by a therapeutically effective amount of the tissue system to restore or regenerate function. As used herein, a “therapeutically effective amount” of the tissue system is an amount sufficient to arrest or ameliorate the physiological effects in a subject caused by the loss, damage, malfunction, or degeneration of limbal stem cells. The therapeutically effective amount of cells or tissues used will depend on the needs of the subject, the subject's age, physiological condition and health, the desired therapeutic effect, the size of the area of tissue that is to be targeted for therapy, the site of implantation, the extent of pathology, the chosen route of delivery, and the treatment strategy. The tissue system is preferably administered to the patient in a manner that permits the tissue system to graft to the intended site and reconstitute or regenerate the functionally deficient area.

In preferred embodiments, the tissue system of the present disclosure is used to therapeutically treat subjects with ocular damage or disease, particular ocular surface impairments. Alternatively, the disclosed tissue system may be used to treat other diseases or damage which will therapeutically benefit from a source of undifferentiated stem cells derived from limbal tissue, for example to repair burned skin areas. The disclosed tissue system is particularly well suited to treat subjects with primary limbal stem cell deficiencies, which may be caused by hereditary conditions such as aniridia, multiple-endocrine-deficiency-associated keratitis, limbitis, and idiopathy, or secondary limbal stem cell loss, which may occur from acquired conditions such as Steven-Johnson syndrome, infections (such as severe microbial keratitis), ocular surface tumors, traumatic destruction of limbal stem cells caused by chemical or thermal injury or exposure to ultraviolet radiation, multiple surgeries or cryotherapies, corneal intraepithelial neoplasia, peripheral ulcerative or inflammatory keratitis, ischemic keratitis, keratopathy, toxic effects induced by contact lens or lens cleaning fluids, immunological conditions, ocular cicatrical pemphigoid, pterygium, pseudopterygium, and the like. In certain embodiments, the tissue system is transplanted, implanted, or grafted to the subject, and is able to repair ocular damage or disease in the subject, for example, by providing a stable limbal stem cell population to the subject's damaged or diseased eye. In certain embodiments, transplantation, implantation, or grafting of the tissue system facilitates epithelization, maintains normal epithelial phenotype, reduces inflammation, reduces scarring, reduces adhesion of tissue, reduces vascularization, and improves vision in the eye.

For example, the tissue system can be transplanted, implanted, or grafted to repair a damaged cornea. While many such methods are well known to those of skill in the art, one such method involves periotomy at the limbus, followed by removal of the perilimbal subconjunctival scar and inflamed tissues to the bare sclera. The fibrovascular tissue of the cornea may be removed by lamellar keratectomy. The tissue system can be scaled according to the size of the recipient eye, and transplanted or grafted to the corresponding recipient limbal area. Alternatively, the tissue system may be used as a whole lamellar corneal tissue, and transplanted or grafted as lamellar keratoplasty to cover the entire area. The transplanted, implanted, or grafted tissue system is then secured to the damaged site, for example with sutures or any other means known to those of skill in the art.

The invention further provides methods of regenerating or repairing tissue in a subject comprising introducing the cell of the LSC population or LSC-like population or the SESC population or SESC-like population of the invention into or onto a subject in a sufficient amount to regenerate or repair tissue.

In one embodiment, the tissue regenerated or repaired comprises tissues of corneal epithelial cell lineage comprising limbal stem or progenitor cell (LSC) and corneal epithelial cell.

The invention additionally provides methods for obtaining limbal stem cell or progenitor (LSC)-like cells from skin epithelial stem cells (SESCs) of a subject. In an embodiment of the invention, the method comprises introduction of a PAX6 gene or up-regulating PAX6 gene expression in SESCs, in order to increase PAX6 protein in SESCs to a sufficient level so as to convert SESCs to LSC-like cells, thereby obtaining LSC-like cells from SESCs of a subject. In one embodiment, introduction of a PAX6 gene or up-regulating PAX6 gene expression in SESCs for obtaining limbal stem or progenitor cell (LSC)-like cells from skin epithelial stem cells (SESCs) of a subject comprises the steps of (a) obtaining SESCs from the subject; (b) culturing the SESCs in a feeder-free cell culture in vitro or ex vivo; (c) introducing at least one PAX6 gene or up-regulating PAX6 gene expression in the SESCS so as to increase PAX6 protein in SESCs to a sufficient level so as to convert SESCs to limbal stem cell or progenitor (LSC)-like cells, thereby obtaining mammalian limbal stem cell or progenitor (LSC)-like cells from skin epithelial stem cells (SESCs) from a subject.

In accordance with the practice of the invention, the methods may be an in vitro method, ex vivo method or in situ or directly applied on a subject.

In one embodiment, the subject is treated with an agent that introduces a nucleic acid encoding PAX6 protein, up-regulates PAX6 gene expression, or increases PAX6 activity. Suitable examples of the agent includes but are not limited to a gene therapy vector, viral particle, lentivirus, adenovirus, adeno-associated virus, recombinant nucleic acid, recombinant protein, PAX6 protein, small molecule regulator of PAX6 expression, inhibitor of a negative regulator of PAX6 expression, a small molecule inhibitor of a negative regulator of PAX activity, a small molecule enhancer of PAX6 activity, or a combination thereof.

Examples of the PAX6 gene includes but are not limited to a PAX6a gene, PAX6b gene, engineered PAX6a gene, engineered PAXb gene, any member of the PAX6 gene family, nucleic acid encoding all or part of PAX6a protein, nucleic acid encoding all or part of PAX6b protein, and any nucleic acid encoding a protein with PAX6 or PAX6-like activity. In one embodiment of the invention, the PAX6 protein is any of a PAX6a protein, PAX6b protein, any member of the PAX6 family of proteins, and any protein with PAX6 or PAX6-like activity. In an embodiment of the invention, PAX6 or PAX6-like activity may involve any protein which can cause increased expression of endogenous K19, wherein the K19 upregulated SECS may differentiate to corneal epithelial cell (CEC) or CEC-like cells with increased expression of K3 and K12 genes and decreased expression of K11 and K10 genes.

In one embodiment, the invention provides a method for obtaining corneal epithelial cell (CEC)-like cells from LSC-like cells of the invention as describe above and, further comprising differentiating cells of (c) in a feeder-free LSC differentiation medium so as to convert LSC-like cells to CEC-like cells.

In an embodiment of the invention, the feeder-free LSC differentiation medium may be chemically defined. In another embodiment of the invention, the medium is xeno-free or free of components other than components derived from the same species as the cultured cells. In yet another embodiment, the medium may be serum-free. In a further embodiment, the medium may be devoid of any animal or human product.

For example, a xeno-free medium or xeno-free culture medium is one in which the medium does not contain a foreign animal-derived product or material. In particular, no product or material in the culture medium is produced in foreign animal cells or contacted foreign animal cells. A culture medium comprising fetal bovine serum would not be considered xeno-free if used to culture human cell or any animal cell other than a bovine cell as serum would be derived from a cow; whereas, a culture medium comprising human serum in the absence of any animal-derived product or material, would be considered xeno-free if culturing a human cell. For example, a xeno-free medium may contain products or materials produced or obtained from micro-organisms, such as bacterium or yeast. A xeno-free medium may also be considered an “animal-free” medium. In this example, xeno-free refers to the absence of foreign animal-derived product or material.

The invention further provides a method for obtaining limbal stem or progenitor cell (LSC)-like cells from skin epithelial stem cells (SESCs). The method may comprise the steps of (a) obtaining SESCs from a subject; (b) culturing the SESCs in a feeder-free cell culture in vitro; (c) contacting the SESCs with an agent to up-regulate PAX6 gene expression in the SESCs so as to increase PAX6 protein in SESCs to a sufficient level to convert SESCs to limbal stem cell or progenitor (LSC)-like cells, thereby obtaining mammalian limbal stem cell or progenitor (LSC)-like cells from skin epithelial stem cells (SESCs) from a subject.

Examples of suitable agents include, but are not limited to, a nucleic acid comprising a PAX6 gene, a gene therapy vector, viral particle, lentivirus, adenovirus, adeno-associated virus, recombinant protein, PAX6 protein, small molecule regulator of PAX6 expression, inhibitor of a negative regulator of PAX6 expression, a small molecule inhibitor of a negative regulator of PAX activity, a small molecule enhancer of PAX6 activity, and a combination thereof.

Examples of suitable PAX6 genes include, but are not limited to, a PAX6a gene, PAX6b gene, engineered PAX6a gene, engineered PAXb gene, any member of the PAX6 gene family, nucleic acid encoding all or part of PAX6a protein, nucleic acid encoding all or part of PAX6b protein, and any nucleic acid encoding a protein with PAX6 or PAX6-like activity.

In accordance with the practice of the invention, the PAX6 protein any be any of a PAX6a protein, PAX6b protein, any member of the PAX6 family of proteins, or any protein with PAX6 or PAX6-like activity and fragment thereof.

The invention further provides an in vitro method for obtaining mammalian LSCs from a subject. In one embodiment, the method comprises the steps of (a) obtaining a sample of tissue from the limbus region of an eye from the subject; (b) dissociating the tissue so as to obtain single cells; and (c) culturing single cells of (b) in a feeder-free cell culture medium so as to permit LSCs to proliferate, wherein the proliferated LSCs have a potential to differentiate into corneal epithelial cells (CECs), thereby obtaining mammalian LSCs from a subject in vitro. In one embodiment, the limbus region comprises the corneal limbus of an eye. In an embodiment of the invention, the step of dissociating the tissue in (b) comprises mechanical or physical disaasociation via an equipment or tool (e.g. a laser) to mechanically dissociate tissue to smaller masses and/or single cells. In another embodiment, dissociating involves the use of an agent or agents such as an enzyme, protease, a chemical, a metal chelator, or combination thereof. Other methods of dissociating a tissue may be used to obtain single LSCs, as is known in the art.

In an embodiment of the invention, the feeder-free cell culture medium further comprises a rho-associated protein kinase (ROCK) inhibitor or leukemia inhibitory factor (LIF) or both. An example of a ROCK inhibitor is Y-27632 (4-[(1R)-1-aminoethyl]-N-4-pyridinyl-trans-cyclohexanecarboxamide, dihydrochloride).

In another embodiment of the invention, the method further comprises the step of converting the LSCs or LSC-like cells to CEC-like cells by culturing on a matrix or an extracellular matrix.

The invention further provides a method for obtaining and/or expanding in vitro mammalian limbal stem or progenitor cells (LSCs) from a subject in a feeder-free LSC culture medium. In one embodiment, the method comprises (a) obtaining a sample of tissue from the limbus region of an eye from the subject; (b) dissociating the tissue so as to obtain single cells; and (c) culturing single cells of (b) in a feeder-free cell culture medium so as to permit LSCs to proliferate, wherein the proliferated LSCs have a potential to differentiate into corneal epithelial cells (CECs), thereby obtaining and expanding in vitro mammalian limbal stem cells from a subject. In an embodiment of the invention, in step (c) of the method, single cells are cultured on a matrix or an extracellular matrix.

In accordance with the practice of the invention, the matrix or extracellular matrix may be any of Matrigel® or its equivalent, growth factor reduced Matrigel® or its equivalent, collagen, collagen IV, collagen IV sheet, mammalian amniotic membrane, human amniotic membrane, fibrinogen, thrombin, perlecan, laminin, fibronectin, recombinant fibronectin, proteoglycan, procollagens, hyaluronic acid, entactin, heparan sulfate, tenascin, poly-L-lysine, gelatin, poly-L-ornithine, extracellular matrix proteins (Fischer or Life Tech), thrombin sheet (Fibrin Sealant, Reliseal™, Reliance Life Sciences), fibrinogen and thrombin sheet (Reliance Life), and any combination thereof.

In another embodiment, the method further comprises step (d), wherein the proliferated LSCs or LSC-like cells are passaged at about 70-90% confluence before passage and about 15-20% confluence after passage. In yet a further embodiment, the number of times the proliferated LSCs or LSC-like cells may be passaged stably as LSCs or LSC-like cells is about 17 or more passages. In another embodiment, the LSCs may proliferate with a generation time of about 16-20 hours. Further, the LSC or LSC like cells may propagate stably for about 40-60 generations without differentiating to CECs. In an embodiment of the invention, the feeder-free LSC culture medium may be changed every other day.

In accordance with the practice of the invention, the limbus region may comprise a corneal limbus of an eye, margin between cornea and conjunctiva, border of cornea and sclera, corneoscleral limbus, a region comprising interpalisade rete ridge, or a region comprising Palisades of Vogt.

In step b, the method further provides, in one embodiment, the step of dissociating the tissue by treating or contacting the tissue with a dissociation agent or agents wherein the dissociation agent or agents is enzyme, protease, a chemical, a metal chelator, or combination thereof. In another embodiment, dissociation is achieved via mechanical or physical disruption via an equipment or tool to mechanically dissociate tissue to smaller masses and single cells.

In one embodiment, the protease may comprises trypsin, collagenase IV, or combination thereof. Further, the metal chelator may comprises EDTA, EGTA, or combination thereof.

In an embodiment of the invention, the feeder-free cell culture medium may comprise a minimum essential medium, a growth factor, a hormone, and a soluble factor. In a further embodiment, the feeder-free cell culture medium may further comprise serum, preferably serum from a species from which the LSCs are being obtained and expanded, or a serum substitute. In a further embodiment, the feeder-free cell culture medium may yet further comprise a rho-associated protein kinase (ROCK) inhibitor. In a further embodiment, the feeder-free cell culture medium additionally comprises Leukemia Inhibitory Factor (LIF).

Suitable examples of ROCK inhibitors include, but are not limited to, (R)-(+)-trans-4-(1-aminoethyl)-N-(4-Pyridyl)cyclohexanecarboxamide dihydrochloride monohydrate (Y-27632), 5-(1,4-diazepan-1-ylsulfonyl) isoquinoline (fasudil or HA 1077), H-1152, H-1152P, (S)-(+)-2-Methyl-1-[(4-methyl-5-isoquinolinyl)sulfonyl]homopiperazine dihydrochloride, Dimethylfasudil (diMF; H-1152P), N-(4-Pyridyl)-N′-(2,4,6-trichlorophenyl)urea, Y-39983, Wf-536, SNJ-1656, and (S)-(+)-2-methyl-1-[(4-methyl-5-isoquinolinyl)sulfonyl]-hexahydro-1H-1,4-diazepine dihydrochloride (H-1152), imidazole-containing benzodiazepines, imidazopyridine derivative, compound comprising an indazole core, a 2-aminopyridine/pyrimidine core, a 9-deazaguanine derivative, benzamide, or aminofurazan, and derivative and analog thereof, and a combination thereof.

In an embodiment of the invention, the ROCK inhibitor is added to the feeder-free cell culture medium after about passage four (4) of LSCs, following isolation of LSCs from the subject.

In one embodiment, the LIF is added to the feeder-free cell culture medium after about passage four (4) of LSCs, following isolation of LSCs from the subject.

In a particular embodiment of the invention, the feeder-free cell culture medium comprises DMEM/F12 medium, DMEM, penicillin-streptomycin, serum, EGF, insulin, hydrocortisone, cholera toxin, 3,3′,5-triiodo-L-thyronine, or combination thereof. The serum may be fetal bovine serum but preferably serum from same species as the LSC being cultured or a serum substitute. The feeder-free cell culture medium may further comprises a ROCK inhibitor, Y-27632. The ROCK inhibitor, Y-27632, may be added to the feeder-free cell culture medium after about cell passage 4. The feeder-free cell culture medium further comprises Leukemia Inhibitory Factor (LIF). The LIF may be added to the feeder-free cell culture medium after about passage four (4) of LSCs, following isolation of LSCs from the subject.

In one embodiment of the invention, the LSCs so obtained and/or expanded may express a set of markers comprising WNT7A, FZD5, PAX6, p63, keratin 5 (K5), keratin 14 (K14), keratin 19 (K19) and Ki67. In another embodiment, about 90-95% of the LSCs so obtained and/or expanded express p63, PAX6, K19 and Ki67. In yet another embodiment, less than about 5% of the LSCs express K5 and K14. In a further embodiment, greater than about 95% of the LSCs express WNT7A and FZD5. In an additional embodiment, the CECs express a set of markers comprising WNT7A, FZD5, PAX6, keratin 3 (K3), and keratin 12 (K12) with statistically significant higher expression of K3 and K12 over LSCs and statistically significant lower expression of K19 over LSCs. In another embodiment, CECs do not express or express significantly lower level of p63 than LSCs and do not express or express significantly lower level of keratin 1 (K1) and keratin 10 (K10) than skin or epidermal cells.

The invention also provides methods for differentiating isolated LSCs to corneal epithelial cells (CECs) in vitro. The method may comprise (a) obtaining previously cultured LSCs, preferably dissociated to a single cell state, which originated initially from a subject; (b) placing isolated LSCs in and/or on a matrix or extracellular matrix to form or allow formation of three dimensional cell culture suitable for differentiation; and (c) culturing LSCs in a LSC differentiation medium so as to permit differentiation of isolated LSCs to CECs in vitro, thereby differentiating isolated LSCs to corneal epithelial cells in vitro.

In accordance with the practice of the invention, the matrix or extracellular matrix may be any of Matrigel® or its equivalent, growth factor reduced Matrigel® or its equivalent, collagen, collagen IV, collagen IV sheet, mammalian amniotic membrane, human amniotic membrane, fibrinogen, thrombin, perlecan, laminin, fibronectin, recombinant fibronectin, proteoglycan, procollagens, hyaluronic acid, entactin, heparan sulfate, tenascin, poly-L-lysine, gelatin, poly-L-ornithine, extracellular matrix proteins (Fischer or Life Tech), thrombin sheet (Fibrin Sealant, Reliscal™, Reliance Life Sciences), fibrinogen and thrombin sheet (Reliance Life), and any combination thereof.

In one embodiment, the matrix or extracellular matrix comprises growth factor reduced Matrigel® or its equivalent, or collagen. In another embodiment, the limbal stem cell differentiation medium is feeder-free and chemically defined medium. For example, in one embodiment, the feeder-free and chemically defined medium may comprise CnT-30 medium (CelIntec Advanced Cell Systems AG, Bern, Switzerland), CnT-02 (CelIntec), CnT-02-3DP5 (CelIntec) or functionally equivalent, wherein the medium promotes differentiation of LSCs to CECs.

In a further embodiment, the CECs so express a set of markers comprising WNT7A, FZD5, PAX6, keratin 3 (K3), and keratin 12 (K12) with statistically significant higher expression of K3 and K12 over LSCs and statistically significant lower expression of K19 over LSCs.

In yet a further embodiment, the CECs so produced, obtain or expanded, do not express or express significantly lower level of p63 than LSCs and do not express or express significantly lower level of keratin 1 (K1) and keratin 10 (K10) than skin or epidermal cells.

In one embodiment of the methods of the invention, the LSC differentiation medium is changed every other day.

The invention also provides a method for obtaining and expanding SESCs in vitro in a feeder-free cell culture medium comprising culturing isolated SESCs skin in the feeder-free cell culture medium of claim of the invention. In one embodiment, the SESCs may be isolated from interfollicular epidermis. In another embodiment, the SESCs are isolated from any SESC niche harboring SESC in a human or animal.

The invention additionally provides a method for obtaining epithelial stem cell (SESC) or SESC-like cells from limbal stem cells (LSCs) comprising: (a) knocking down expression or activity of WNT7A or PAX6 gene or protein sufficiently so as to change the cell fate from LSC to SESC fate, thereby obtaining SESC or SESC-like cells from LSC cells. In one embodiment, the LSCs may be contacted with an shRNA directed to WNT7A or PAX6, or alternatively, LSCs express an introduced gene producing shRNA, RNAi or anti-sense RNA directed to WNT7A or PAX6 gene so as to sufficiently reduce expression of WNT7A or PAX6 to convert LSCs to SESC or SESC-like cells.

The invention also provides a method for obtaining and expanding skin epithelial stem cell (SESC) in vitro from a subject in a feeder-free cell culture medium. In one embodiment the method comprises: (a) obtaining a sample of tissue from the interfollicular epidermis or any SESC stem cell niche harboring SESCs in the subject; (b) dissociating the tissue so as to obtain single cells; and (c) culturing single cells of (b) in a feeder-free cell culture medium so as to permit SESCs to proliferate, wherein the proliferated SESCs have a potential to differentiate into skin epidermal cells, thereby obtaining and expanding in vitro skin epithelial stem cells in vitro from a subject. In another embodiment, the SESCs may be cultured in vitro in a feeder-free cell culture medium of the invention.

The invention further provides a method for obtaining skin epidermal cells or skin epidermal like cells from SESC or SESC-like cells in vitro comprising culturing isolated SESC or SESC-like cells in chemically defined differentiation medium which support differentiation of SESC or SESC-like cells to skin epidermal cells or skin epidermal-like cells, thereby obtaining skin epidermal cells or skin epidermal-like cells from SESC or SESC-like cells in vitro. In one embodiment, the chemically defined differentiation medium may be CnT-02 (CelInTec) or equivalent medium.

The invention yet further provides a method for treating a subject in need of new skin, wherein the method comprises administering SESC cells, SESC-like cells, skin epidermal cells or skin epidermal-like cells obtained by the method of the invention or obtained and expanded by any of the methods of the invention to the subject, such as to repopulate SESC cell population or skin epidermal cell population of the subject and provide new skin for the subject, thereby treating a subject in need of new skin.

In one embodiment, the subject in need of skin may suffers from skin dystrophy, skin disease, skin infection, burn injury, skin ulcer, abrasion, melanoma, carcinoma, wound, aging, genetic disorder of the skin, skin biopsy, surgery, cosmetic defect, or reconstructive surgery affecting the skin. Alternatively, the subject in need of skin may be a subject needing skin replacement or undergoing cosmetic surgery or reconstructing surgery affecting the skin.

The invention additionally provides a method of changing a limbal stem or progenitor cell (LSC) and/or its progeny to a SESC or SESC-like cell. The method comprises downregulating expression of WNT7A or PAX6 gene in the LSC sufficiently so as to change the LSC and/or its progeny to SESC or SESC-like cell, thereby changing the LSC and/or its progeny to a SESC or SESC-like cell.

The invention also provides a method of changing a LSC-like cell and/or its progeny to a SESC or SESC-like cell. The method comprises downregulating expression of WNT7A or PAX6 gene in the LSC-like cell sufficiently so as to change the LSC-like cell and/or its progeny to SESC or SESC-like cell, thereby changing the LSC-like cell and/or its progeny to a SESC or SESC-like cell.

The invention provides a method of changing a skin epithelial stem cell (SESC) and/or its progeny to a LSC or LSC-like cell. The method comprises upregulating or overexpressing PAX6 or WNT7A in the SESC sufficiently so as to change the SESC cell and/or its progeny to LSC or LSC-like cell, thereby changing the SESC and/or its progeny to a LSC or LSC-like cell.

Additionally, the invention provides a method of changing a SESC-like cell and/or its progeny to a LSC or LSC-like cell. The method comprises upregulating or overexpressing PAX6 or WNT7A in the SESC-like cell sufficiently so as to change the SESC-like cell and/or its progeny to LSC or LSC-like cell, thereby changing the SESC-like and/or its progeny to a LSC or LSC-like cell.

The invention provides a population of isolated limbal stem cells (LSCs) or LSC-like cells derived or produced from any of the methods of the invention. In one embodiment, the LSCs express a set of markers comprising WNT7A, FZD5, PAX6, p63. keratin 5 (K5). keratin 14 (K14), keratin 19 (K19), and Ki67.

The invention further provides a population of isolated corneal epithelial cells (CECs) derived from any of the methods of the invention. In one embodiment, the CECs express (i) a set of markers comprising WNT7A, FZD5, PAX6, keratin 3 (K3) and keratin 12 (K12) with statistically significant higher expression of K3 and K12 than LSCs and statistically significant lower expression of K19 than LSCs, and (ii) do not express or express significantly lower level of p63 than LSCs and do not express or express significantly lower level of keratin 1 (K1) and keratin 10 (K10) than skin or epidermal cells.

The invention further provides a kit for corneal tissue repair comprising LSCs, LSC-like cells, or CECs produced by any of the methods of the invention, or combination of said cells, a packing material and an instruction for use. In one embodiment, the kit further comprises a pharmaceutically acceptable carrier. The pharmaceutically acceptable carrier may be a contact lens or its equivalent used to support cell attachment or growth in a curvature as a curvature of a human or animal eye. In another embodiment, the kit further comprises human amniotic membrane or animal amniotic membrane.

The invention additionally provides a pharmaceutical composition comprising an effective amount of LSCs, LSC-like, CECs or CEC-like cells produced by any of the methods of the invention, or combination of said cells thereof, and a suitable pharmaceutical carrier.

The invention also provides a kit for growing and maintaining LSC or LSC-like population of the invention or SESC or SESC-like population of the invention comprising a cell culture medium for maintaining LSC population, wherein the medium is essentially feeder-free. In one embodiment, the components of the kit of is essentially xeno-free. In yet another embodiment, the kit further comprises a LSC differentiation medium for differentiating LSC or LSC-like population to CEC or CEC-like population. In a further embodiment, the components of the kit is essentially serum-free. In yet a further embodiment, the components are essentially free of animal product, such as essentially free of human product. In an additional embodiment, the components of the kit are chemically defined.

Also provided is a kit for corneal tissue repair comprising LSCs, LSC-like, CECs or CEC-like cells produced by any of the methods of the invention, or combination of said cells, a packing material and an instruction for use. The kit may further comprise a pharmaceutically acceptable carrier. The pharmaceutically acceptable carrier is a contact lens or its equivalent used to support cell attachment or growth in a curvature as curvature of a human or animal eye. Additionally, the kit may further comprise human amniotic membrane or animal amniotic membrane.

Therapeutic Uses

The invention provides methods for treating a subject with a disease associated with malfunctioning limbal stem cells or corneal epithelial cells. In one embodiment, the method comprises: (a) transplanting LSCs, LSC-like. CECs or CEC-like cells of the invention, or produced by the methods of the invention to an affected eye of a subject, wherein the transplanted cells populate cornea or limbus of the affected eye of the subject and restore normal cornea clarity and transparency, thereby treating the subject with the disease associated with malfunctioning limbal stem or progenitor cells or corneal epithelial cells.

Examples of the disease or condition include, but are not limited to, a deficiency of limbal stem or progenitor cells, a deficiency of corneal epithelial cells, damage to corneal limbus, damage to cornea of an eye, damage to limbal stem cells, damage to corneal epithelial cells, congenital defect affecting corneal development or function, acquired defect affecting corneal development or function, congenital defect affecting cell fate determination switching corneal to skin lineage, acquired defect affecting cell fate determination switching corneal to skin lineage, abnormal epidermal differentiation, Stevens-Johnson syndrome, aniridia, recurrent pterygium, corneal disease, corneal epithelium squamous metaplasia, inflammatory keratopathy, trauma, chemical burns, alkaline burn, partial blindness, or complete blindness.

In one embodiment, the disease or condition is a partial blindness, complete blindness, corneal surface disease, corneal disease, corneal epithelium squamous metaplasia, inflammatory keratopathy, trauma or alkaline burn.

The invention further provides methods for restoring normal cornea clarity and transparency of a subject with partial blindness, complete blindness, corneal surface disease, corneal disease, corneal epithelium squamous metaplasia, inflammatory keratopathy, trauma or alkaline burn. In one embodiment, the method comprises (a) transplanting LSCs, LSC-like, CECs or CEC-like cells of the invention or produced by the methods of the invention to an affected eye of the subject, wherein the transplanted cells populate cornea or limbus of the affected eye of the subject and restore normal cornea clarity and transparency, thereby restoring normal cornea clarity and transparency of a subject with partial blindness, complete blindness, corneal surface disease, corneal disease, corneal epithelium squamous metaplasia, inflammatory keratopathy, trauma or alkaline burn.

The invention also provides a method of regenerating or repairing corneal tissue in a subject comprising introducing isolated LSC, LSC-like, CECs or CEC-like cell population of the invention, or produced by any of the methods of the invention into the subject in a sufficient amount to regenerate or repair corneal tissue.

In one embodiment, the cells are from an individual other than the subject. In another embodiment, the cells are from the subject being treated with cells produced by the said method.

In one embodiment, the subject is a mammal. Examples of mammals include, but are not limited to, a human, rat, dog, cat, pig, horse, rabbit, cow, monkey or mouse.

The invention further provides a method to determine if a patient with ocular metaplasia may benefit from treatment with PAX6 gene or gene product. In one embodiment, the method comprises assessing gene expression or protein level of WNT7A, PAX6, K3 and/or K12 in area of metaplasia, absence of WNT7A, PAX6, K3 and/or K12 in area of metaplasia indicates the patient with ocular metaplasia may benefit from treatment with PAX6 gene or gene product or cells of the invention.

Additionally, the invention further provides a method to determine if a patient with ocular metaplasia may benefit from treatment with WNT7A gene or gene product. In an embodiment of the invention, the method comprises assessing gene expression or protein level of WNT7A, PAX6, K3 and/or K12 in area of metaplasia, absence of WNT7A, PAX6, K3 and/or K12 in area of metaplasia indicates the patient with ocular metaplasia may benefit from treatment with WNT7A gene or gene product.

Also, the invention further provides a method to determine if a patient with ocular metaplasia may benefit from treatment with both WNT7A and PAX6 genes or gene products. In one embodiment, the method comprises assessing gene expression or protein level of WNT7A, PAX6, K3 and/or K12 in area of metaplasia, absence of WNT7A, PAX6, K3 and/or K12 in area of metaplasia indicates the patient with ocular metaplasia may benefit from treatment with both WNT7A and PAX6 genes or gene products.

Further, the invention provides a method to determine if a patient with ocular metaplasia may benefit from treatment with PAX6 gene or gene product or cells produced by any of the methods of invention, wherein the method comprises assessing gene expression or protein level of WNT7A, PAX6, K3 and/or K12 in area of metaplasia, absence of WNT7A, PAX6, K3 and/or K12 in area of metaplasia indicates the patient with ocular metaplasia may benefit from treatment with PAX6 gene or gene product or cells produced by any of the methods of the invention.

The invention also provides a method of regenerating or repairing corneal tissue in a subject comprising introducing the isolated LSC population of the invention, into the subject in a sufficient amount to regenerate or repair corneal tissue. The subject may be a mammalian subject. Examples include but are not limited to a human, rat, dog, cat, pig, horse, rabbit, cow, monkey or mouse.

Further, the invention additionally provides a method for treating an eye disease comprising administering the isolated LSC or LSC-like population of the invention or SESC or SESC-like population of the invention to a subject in a sufficient amount so that the isolated LSC or LSC-like population of the invention produces or overexpresses PAX6 in a sufficient amount so as to produce or maintain LSC or LSC-like state permitting differentiation to CEC or CEC-like cells, thereby treating the eye disease. In one embodiment, the eye disease is a human corneal disease, corneal epithelium squamous metaplasia, inflammatory keratopathy, trauma and alkaline burn. In accordance with the practice of the invention, the eye disease may be a human eye disease.

Additionally provided are methods of regenerating or repairing corneal tissue in a subject comprising contacting corneal tissue, presumptive location of corneal tissue or anterior surface of an eye of a subject with PAX6 in a sufficient amount to regenerate or repair corneal tissue.

Additionally provides are methods of regenerating or repairing corneal tissue in a subject comprising administering LSCs, LSC-like, CECs or CEC-like cells produced by any of the methods of the invention or combination of said cells thereof, on a cornea or anterior surface of an eye in a sufficient amount to regenerate or repair corneal tissue.

Further provided are methods of regenerating or repairing corneal tissue in a subject comprising administering PAX6 protein onto corneal tissue, presumptive location of corneal tissue or anterior surface of an eye in a sufficient amount to regenerate or repair corneal tissue.

Also provided are methods for assessing risk of developing an eye disease affecting cornea or cornea function in a subject. In one embodiment said method comprises (a) assessing activity of WNTZ7A, FZD5 and PAX6 or combination thereof, in LSCs or corneal epithelial cells, wherein lower activity or no activity of WNTZ7A, FZD5 and PAX6 or combination thereof indicates a higher risk of developing an eye disease affecting the cornea or cornea function in a subject, thereby assessing the risk of developing an eye disease affecting cornea or cornea function in a subject.

The invention further provides a method for identifying a patient population suitable for cell-transplantation with LSCs, LSC-like, CECs or CEC-like cells produced by any of the methods of the invention, or combination of said cells thereof, or treatment with PAX6 protein or PAX6-encoding nucleic acid, wherein the patient population has abnormal skin epidermal-like cells in the cornea and associated keratinization and loss or decreased expression of WNTZ7A, FZD5 and PAX6 genes or combination of gene(s).

The invention also provides methods for repopulation limbus with LSC or LSC-like cells in a subject with a deficiency of limbus stem cells. In one embodiment, the method comprises administering LSC or LSC-like cells produced by any of the methods of the invention to the anterior surface of an eye of a subject and permitting the cells to migrate to a LSC niche, thereby repopulation the limbus with LSC or LSC-like cells in the subject. In another embodiment, the method comprises administering LSC or LSC-like cells comprises grafting a sheet or sheets of LSC or LSC-like cells onto a surface of an eye of the subject, or alternatively, administering a cell or tissue suspension comprising LSC or LSC-like cells. In a further embodiment, after administering LSC or LSC-like cells to an eye of the subject, the LSC or LSC-like cells may be covered with an amniotic membrane, preferably human amniotic membrane.

The invention additionally provides a method for repopulating limbus with LSC or LSC-like cells in a subject with a deficiency of limbus stem cells comprising administering an agent that increases PAX6 activity or expression to an area of conjunctiva, presumptive corneal location, eye or eyelid in a subject so as to convert skin epithelial stem cells (SESCs) in the conjunctiva, presumptive corneal location, eye or eyelid to LSC or LSC-like cells which upon migration to the limbus repopulates the limbus with LSC or LSC-like cells in the subject, thereby repopulating the limbus with LSC or LSC-like cells in a subject.

Accordingly, in one aspect, the invention is directed to a supplying the limbal stem cells of the invention for treating, e.g., visual system and other organ injuries or diseases. Exemplary visual system injuries or diseases that the therapeutic composition of the invention may be used to treat are as follows: for the reconstruction of the ocular surface in patients with limbal stem cells deficiency (Tseng et al., 1998); for the treatment of visual system age-related diseases in general; for reconstruction of the ocular surface in patient with corneal persistent epithelial defect (Tseng et al., 1998); for corneal epithelial healing and to avoid corneal stromal remodeling and haze formation after photorefractive keratectomy (Woo et al., 2001); as a substance that can promote and support healing processes following ocular surface damage related to Stevens Johnson Syndrome and OCP (Tsubota et al., 1996); for healing support and a therapeutic approach in other eye anterior surface diseases including dry eye, Sjögren's syndrome, thermal and chemical burns, and acute and chronic inflammation; and as a versatile compound that can treat the causes of total and partial epithelial stem cells deficiency. Exemplary total epithelial stem cell deficiencies include, but are not limited to, chemical and thermal injuries, Stevens Johnson Syndrome, multi-surgery effects in the limbal region, contact lens over-wear and severe microbial infections. Exemplary partial epithelial stem cell deficiency include, but are not limited to, neurotrophic keratitis, ischemic keratitis, peripheral ulcerative and inflammatory keratitis, limbitis, aniridia, pterigium, pseudopterigium and multiple endocrine deficiency (Tseng et al., 1998; Uchida et al., 2000).

Other exemplary uses of the composition according to the invention are as a treatment for skin dystrophies, burn injury and skin ulcers (Trelford et al., 1979); as a therapy for chemiotheraphic stomatitis; as an immunomodulator in autoimmune disease; to increase tolerance in the treatment of auto-, allo- and xeno-transplants; as an osteoinductive property substance for guided bone regeneration (Gomes et al., 2001); as a substance that can be incorporated in the actual hardware currently used for bacterial and other simple organism culture in vitro or in vivo; as a substance that can be incorporated in currently used devices dedicated to cell culture, such as cell culture dishes, a three-dimensional matrix or a gel (Uchida et al., 2000); as a storage or culture medium for human cells; as a part of an integrated delivery system that will transport the effective compound from an accessible site to the site in need, for remote release of all the beneficial effects of the amniotic membrane; as a bone and tissue anti-inflammatory drug; as a source of factors and receptors for used in neuro-degenerative or inflammatory diseases; and as a source of receptors that mediate glucose transport.

In another embodiment of the method, said ocular surgical procedure changes the shape of the cornea or is refractive surgery. In a specific embodiment, said ocular surgical procedure is photorefractive keratectomy (PRK), laser-assisted sub-epithelial keratectomy (LASEK) or laser-assisted in situ keratomileusis (LASIK). In another specific embodiment, said ocular surgical procedure is automated lamellar keratoplasty (ALK), laser thermal keratoplasty (LTK), or conductive keratoplasty (CK).

D. Targeted Diseases and Conditions

The present invention contemplates methods of treating a subject with an eye disease or condition that includes administering to the subject a composition that includes a therapeutic amount of limbal stem cells as set forth herein in a therapeutic preparation suitable for delivery to the subject. The corneal damage may be any injury, condition or disease that has been implicated to play a role in the pathophysiology. Non-limiting examples are set forth below.

1. Ocular Injuries

Corneal wounds are injuries to the ocular surface and can be thermal (i.e. burn), chemical (i.e. acid), physical (i.e. abrasions) or surgical wounds (i.e. corneal transplant) or a combination thereof.

The compositions and methods of the present invention may be effective in treating corneal wounds.

a. Foreign Bodies

About 25% of all ocular injuries involve foreign bodies on the surface of the cornea. No scarring will occur if the injury affects only the corneal epithelium; but if it also affects the Bowman zone, scarring is possible. After removal of the foreign body, the eye is treated with a sulfonamide or antibiotic and, if there is ciliary congestion and photophobia, or if the removal of the foreign body were difficult, it is treated with a cycloplegic such as about 5% homatropine. In some instances, the therapeutic compositions of the present invention are designed to accelerate healing of the injury caused by the foreign body and to prevent infection, and to improve the clinical outcome.

b. Chemical Burns

Chemical burns are treated by first diluting the chemical by flushing the eye with fluid, and then preventing infection through the use of topical antibiotics.

Intraocular pressure may be reduced by applying timolol, epinephrine, acetazolamide, or other similar agents. If epithelialization of the cornea is incomplete after one week, there is a danger of stromal necrosis, in addition to the risk of infection. It is therefore critical that the healing be accelerated to reduce these risks.

Severe scarring is another common result of chemical burns. The therapeutic compositions of the present invention are designed to accelerate the healing of the corneal erosion caused by the chemical burns, to prevent stromal necrosis and infection of the eye, and to reduce corneal scarring and thereby restore/preserve corneal transparency.

Unexpectedly the compositions of the present invention are able to prevent or reduce scar formation while simultaneously enhancing ocular healing, wound repair, and maintaining corneal transparency. While not wishing to be bound by any specific mechanism of action, it appears that these beneficial effects can be obtained due to the anti-inflammatory actions of the compositions. In some instances, the beneficial effects can be obtained due to the combination of anti-inflammatory and anti-apoptotic actions of the compositions of the invention.

c. Lacerations

Lacerations of the cornea are followed by prolapse of the iris, which closes the injury. As in all eye injuries, there is a risk of infection. Lacerations also may extend to the sclera, which is a much more severe injury. In such a case, surgery is required to remove prolapsed uveal tissue from the injured area, and the sclera is closed with sutures. The therapeutic compositions of the present invention are designed to accelerate the healing of the laceration and to prevent infection.

Inflammatory Conditions

Keratitis refers to inflammation of the cornea. Causes include but are not limited to amoebic, bacterial, fungal or viral infection, photokeratitis, exposure (eyelid dysfunction), chemical injury, trauma, surgery (LASIK, PRK, cataract, corneal transplant, pterygium surgery), or congenital causes such as keratoconus, Fuchs' dystrophy, or keratoconjunctivitis sicca. Other Inflammation-mediated conditions of the eye include but are not limited to uveitis, macular edema, age-related macular degeneration, retinal detachment, ocular tumors, multifocal choroiditis, diabetic uveitis, proliferative vitreoretinopathy (PVR), sympathetic opthalmia, Vogt Koyanagi-Harada (VKH) syndrome, histoplasmosis, and uveal diffusion.

Corneal ulcers form when the surface of the cornea is damaged or compromised in some way. The ulcers may be sterile or infected and determines the course of treatment. Bacterially infected ulcers tend to be extremely painful and are typically associated with a break in the epithelium, the outermost layer of the cornea. Certain types of bacteria, such as Pseudomonas, are extremely aggressive and can cause severe damage and even blindness within 24-48 hours if left untreated. Sterile ulcers cause little if any pain. They are often found near the peripheral edge of the cornea and are not necessarily accompanied by a break in the epithelial layer of the cornea. There are many causes of corneal ulcers. Contact lens wearers are at an increased risk of corneal ulcers if they are not diligent in the cleaning, handling, and disinfection of their lenses and cases. Bacterially infected ulcers are also associated with diseases that compromise the corneal surface, creating a window of opportunity for organisms to infect the cornea. Patients with severely dry eyes, who have difficulty blinking, or are unable to care for themselves, are also at risk. Other causes of ulcers include herpes simplex viral infections, inflammatory diseases, corneal abrasions or injuries, and other systemic diseases. The compositions and methods of the present invention may be effective in treating corneal ulcers.

The compositions and methods of the present invention may be effective in treating corneal inflammation. Suspensions of microspheres may be used as an anti-inflammatory therapy of the eye, especially for treating inflammatory conditions of the ocular adnexa, palpebral or bulbar conjunctiva, cornea and anterior segment of the globe. Common therapeutic applications for anti-inflammatory suspensions of microspheres include viral, allergic conjunctivitis, acne rosacea, iritis and iridocyclitis. Microspheres may also be used to ameliorate inflammation associated with, corneal injury due to chemical or thermal burns, or penetration of foreign bodies. Such conditions may result from surgery, injury, allergy or infection to the eye and can cause severe discomfort.

Notably, microspheres have considerable therapeutic advantages in reducing inflammatory responses, compared to the prevalent topical ocular use of NSAI agents and corticosteroids. Use of topical steroids is associated with a number of complications, including posterior subcapsular cataract formation, elevation of intraocular pressure, secondary ocular infection, retardation of corneal wound healing, uveitis, mydriasis, transient ocular discomfort and ptosis. Numerous systemic complications also may arise from the topical ocular application of corticosteroids. These complications include adrenal insufficiency, Cushing's syndrome, peptic ulceration, osteoporosis, hypertension, mLSCle weakness or atrophy, inhibition of growth, diabetes, activation of infection, mood changes and delayed wound healing.

3. Ocular Surgical Applications

Compositions of microspheres in accordance with the present invention, may also be used to ameliorate inflammation associated with ocular surgery, and in this context are particularly useful in a prophylactic modality as well as in promoting healing and reducing scarring as has been detailed above.

Of particular suitability is the use of the compositions of the invention for: photorefractive keratectomy (PRK), laser-assisted sub-epithelial keratectomy (LASEK), laser-assisted in situ keratomileusis (LASIK), automated lamellar keratoplasty (ALK), laser thermal keratoplasty (LTK), conductive keratoplasty (CK), post trabeculectomy (filtering surgery); post-pterygium surgery; post ocular adnexa trauma and surgery; post intraocular surgery and specifically: post lensectomy, post vitrectomy, post retinal detachment surgery, and post epi- and subretinal membrane peeling.

Other corneal pathologies which can be treated include therapeutic photokeratectomy (aka phototherapeutic keratectomy (PTK)), which include complications due to Reis-Bueckler's dystrophia, Groenouw's palindromia, leukoma, post-therapetic keratitis, pterygium, corneal foreign body, several keratopathies, noduli post-keratoplasty, corneal erosion, alkali burning, post-radial keratoplasty and PRK, as well as in the treatment of visual impairment or irritative symptoms related to corneal scars, opacities, or dystrophies extending beyond the epithelial layer (e.g., persistent epithelial defects from anterior stroma dystrophies), recurrent corneal erosions, superficial corneal dystrophy, epithelial membrane dystrophy, corneal melt, corneal ulcers and irregular corneal surfaces due to Salzmann's nodular degeneration or keratoconus nodules.

It will be appreciated by the artisan that these are intended to serve as non-limitative examples of prevalent surgical procedures for which the compositions and methods of the invention are useful.

4. Other Anterior Ocular Conditions

An anterior ocular condition is a disease, ailment or condition which affects or which involves an anterior (i.e., front of the eye) ocular region or site, such as a periocular mLSCle, an eyelid or an eyeball tissue or fluid which is located anterior to the posterior wall of the lens capsule or ciliary mLSCles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Other diseases or disorders of the eye which may be treated in accordance with the present invention include, but are not limited to, ocular cicatricial pemphigoid (OCP), Stevens Johnson syndrome and cataracts.

Dry eye syndrome is one of the most common problems treated by eye physicians. It is usually caused by a problem with the quality of the tear film that lubricates the eyes. Tears are comprised of three layers. The mucus layer coats the cornea, forming a foundation so the tear film can adhere to the eye, the middle aqueous layer provides moisture and supplies oxygen and other important nutrients to the cornea, and the outer lipid layer is an oily film that seals the tear film on the eye and helps to prevent evaporation. Tears are formed by several glands around the eye. The water layer is produced in the lacriminal gland located under the upper eyelid and several smaller glands in the lids make the oil and mucus layers. With each blink, the eyelids spread the tears over the eye. Excess tears flow into two tiny drainage ducts in the corner of the eye by the nose. These ducts lead to tiny canals that connect to the nasal passage. Dry eye syndrome has many causes. One of the most common reasons for dryness is the normal aging process. Many other factors, such as hot, dry or windy climates, high altitudes, air-conditioning and cigarette smoke also cause dry eyes. Many people also find their eyes become irritated when reading or working on a computer. Contact lens wearers may also suffer from dryness because the contacts absorb the tear film, causing proteins to form on the surface of the lens. Certain medications, thyroid conditions, vitamin A deficiency, menopause and diseases such as Parkinson's and Sjogren's can also cause dryness. The compositions and methods of the present invention may be effective in treating dry eye syndrome.

Formulation, Dosage and Administration

Compositions comprising limbal stem cells may be administered to a subject to provide various cellular or tissue functions, for example, to treat ophthalmic disorders due to trauma, surgery, genetics, disease, etc. As used herein “subject” may mean either a human or non-human animal.

Such compositions may be formulated in any conventional manner using one or more physiologically acceptable carriers, optionally comprising excipients and auxiliaries. Proper formulation is dependent upon the route of administration chosen. The compositions may be packaged with written instructions for their use in treating ophthalmic disorders or restoring a therapeutically important metabolic function. The compositions may also be administered to the recipient in one or more physiologically acceptable carriers.

Treatment Kits—The invention also provides for an article of manufacture comprising packaging material and a pharmaceutical composition of the invention contained within the packaging material, wherein the pharmaceutical composition comprises LSCs alone or in combination with a carrier. The packaging material comprises a label or package insert which indicates that the LSC can be used for treating ophthalmic disorders, for example, corneal disorders/diseases/injuries.

In one embodiment, the present invention provides a carrier such as a contact lens product, comprising a soft disposable contact lens loaded with the invention LSC. In one embodiment, the contact lens can be biocompatible lattice. As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material or biocoated to support LSC growth or adherence. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures. The LSC can be expanded on the lens or placed on lens prior to transport to recipient.

Suitable materials for forming soft contact lenses using the method of the invention include, without limitation, silicone elastomers, silicone-containing macromers including, without limitation, those disclosed in U.S. Pat. Nos. 5,371,147, 5,314,960, and 5,057,578 incorporated in their entireties herein by reference, hydrogels, silicone-containing hydrogels, and the like and combinations thereof. More preferably, the lens is made from a material containing a siloxane functionality, including, without limitation, polydimethyl siloxane macromers, methacryloxypropyl polyalkyl siloxanes, and mixtures thereof, a silicone hydrogel or a hydrogel made of monomers containing hydroxy groups, carboxyl groups, or both and combinations thereof. Materials for making soft contact lenses are well known and commercially available. For example, the lens material is acquafilcon, etafilcon, genfilcon, lenefilcon, balafilcon, lotrafilcon, or galyfilcon. The lens may be further enhanced by using additives in the packing solution. An example of such an additive is polyvinylpyrollidine. A suitable lens material is polymethyl methacrylate. However, other materials, such as gas permeable materials (including silicone, a combination of polymethyl methacrylate and silicone, and cellulose acetate butyrate), may also be used.

The contact lenses of the present disclosure may be transparent (i.e., having a transmission of visible light of at least about 20%), opaque or a combination of both. For example, in certain embodiments, the contact lens of the present disclosure may be transparent, in which case the contact lens may or may not provide vision correction for one or both eyes.

The contact lenses of the present disclosure may comprise a soft, flexible material or may comprise a rigid, gas permeable, material. Examples of soft flexible materials may be formed using one or more polymers such as combination of polymers such as silicones, silicone hydrogels or other hydrogel materials (e.g., materials containing homopolymers or copolymers of two or more hydrogel monomers, such as 2-hydroxy ethyl methacrylate, i-vinyl-2-pyrrolidone, methacrylic acid, etc.). Examples of rigid materials include silicone acrylate (S/A) copolymers, fluorosilicone acrylate (F-S/A) copolymers, and poly(methyl methacrylate) (PMMA).

The lenses may have a curvature so as to match the natural curvature of the cornea and provide a standard fit. In the case of a soft contact lens, this may be one size. For example, the contact lenses may be provided in a range of standard corneal curvatures (e.g., a base curve ranging 8 mm to 10 mm, among other values) and may have a range of diameters (e.g., a diameter ranging from 8 mm to 18 mm, among other values) to provide a comfortable and safe fit for the patient. An ideal size for a soft contact lens may be a base curve of 8.8 mm and a diameter of 14 mm, among other values.

Still further aspects of the present disclosure pertain to kits that are useful for treating a patient. The kits may include all or a subset of all the components useful for treating a patient in accordance with the present disclosure. The kits may include, for example: (a) one or more contact lenses in accordance with the present disclosure, (b) a therapeutic amount of cultured LSC of the invention, (c) extracellular matrix in a formulation to support growth of the LSC of the invention, which may or may not be biocoated on the contact lens, (d) optionally, medium to sustain the growth of the LSC of the invention or buffer the LSC during transport, (e) instructions for administering the invention compositions to a patient's eye and/or for fitting the contact lens, and (f) packaging and information as required by a governmental regulatory agency that regulates cell therapy products, pharmaceuticals and/or medical devices. Additionally, the kit of the invention can comprise one or more containers of a suitable liquid carrier (e.g. sterile water for injection, physiological saline, phosphate buffer, phosphate buffered saline, etc.) to wash excess medium from the lens of the invention.

In certain embodiments, the components of the kits are provided in a single sterile package for convenient use by a health care professional.

In preferred embodiments, the matrix is selected from mammalian amniotic membrane, Matrigel™ and its equivalents, laminin, tenascin, entactin, hyaluron, fibrinogen, thrombin, collagen-IV, collagen-IV sheet, poly-L-lysine, gelatin, poly-L-ornithine, fibronectin, thrombin sheet (Fibrin Sealant, Reliseal™, Reliance Life Sciences), and the like, or combinations thereof.

One example of the tissue base for use in generating the tissue system disclosed herein is human amniotic membrane, which may be prepared using methods well known to those of skill in the art. The human amniotic membrane may be used intact with the epithelial surface, or denuded of epithelial cells. Preferred methods for preparing the human amniotic membrane are disclosed in the Examples below. In other embodiments, the tissue base is biocoated with an additional support material, for example a material that facilitates binding of LSCs onto the tissue base. The additional support material that may be employed can be selected from fibrinogen, laminin, collagen IV, tenascin, fibronectin, collagen, bovine pituitary extract, EGF, hepatocyte growth factor, keratinocyte growth factor, hydrocortisone, or combinations thereof.

Alternatively, in a non-limiting embodiment, the compositions of the present invention may be administered to the eye topically, such as, for example, in the form of eye drops. In a further non-limiting embodiment, eye drops includes the LSCs of the invention, which can be administered to the cornea in order to treat a disease or disorder of the cornea.

In various embodiments, compositions of the invention can comprise a liquid comprising the iLSC of the invention in solution, in suspension, or both. As used herein, liquid compositions include gels.

In other embodiments, the tissue base is a collagen gel or a fibrin gel, and the gel may further comprise other desirable cell types for generating the tissue system, including but not limited to fibroblasts, such as corneal stromal fibroblasts, derivatives of mesenchymal tissue, and epithelial cells, such as corneal epithelial cells. In still other embodiments, the tissue base is a hydrogel, for example a synthetic hydrogel, a soft hydrogel contact lens or a poly-HEMA matrix. In certain embodiments, the tissue base will be gradually resorbed in vivo after transplant, implant, or graft of the tissue system. In addition, the tissue base is exemplarily non-antigenic, and facilitates epithelialization without significant fibrovascular growth.

The term “suspension” herein includes a liquid composition, wherein LSC of the invention is present in solution in combination with extracellular matrix and medium. The present invention can also separate the different components of the invention, wherein LSC in suspension with medium and separately a second solution with the extracellular matrix are provided, wherein both suspension and solution are combined at the point of treatment, either introduced separately or in combination (e.g., premixed prior to delivery).

In preferred embodiments, the matrix is selected from mammalian amniotic membrane. Matrigel™ and its equivalents, laminin, tenascin, entactin, hyaluron, fibrinogen, thrombin, collagen-IV, collagen-IV sheet, poly-L-lysine, gelatin, poly-L-ornithine, fibronectin, platelet derived growth factor (PDGF), thrombin sheet (Fibrin Sealant, Reliseal™, Reliance Life Sciences), and the like, or combinations thereof.

One example of the tissue base for use in generating the tissue system disclosed herein is human amniotic membrane, which may be prepared using methods well known to those of skill in the art. The human amniotic membrane may be used intact with the epithelial surface, or denuded of epithelial cells. Exemplary methods for preparing the human amniotic membrane are disclosed in the Examples below. In other embodiments, the tissue base is biocoated with an additional support material, for example a material that facilitates binding of LSCs onto the tissue base. The additional support material that may be employed is preferably selected from fibrinogen, laminin, collagen IV, tenascin, fibronectin, collagen, bovine pituitary extract, EGF, hepatocyte growth factor, keratinocyte growth factor, hydrocortisone, or combinations thereof.

Preferably the liquid composition is aqueous. Alternatively, the composition can take the form of an ointment. In a preferred embodiment, the composition is an in situ gelable aqueous composition, for example as an in situ gelable aqueous solution. Such a composition can comprise a gelling agent in a concentration effective to promote gelling upon contact with the eye or lacrimal fluid in the exterior of the eye.

Aqueous compositions of the invention have ophthalmically compatible pH and osmolality. These compositions can incorporate means to inhibit microbial growth, for example through preparation and packaging under sterile conditions and/or through inclusion of an antimicrobially effective amount of an ophthalmically acceptable preservative. Suitable preservatives non-restrictively include mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

Suitable gelling agents non-restrictively include thermosetting polymers, such as tetra-substituted ethylene diamine block copolymers of ethylene oxide and propylene oxide (e.g., poloxamine 1307); polycarbophil; and polysaccharides, such as gellan, carrageenan (e.g., kappa-carrageenan and iota-carrageenan), chitosan and alginate gums. The phrase “in situ gelable” includes not only liquids of low viscosity that can form gels upon contact with the eye or with lacrimal fluid in the exterior of the eye, but also more viscous liquids such as semi-fluid and thixotropic gels that exhibit substantially increased viscosity or gel stiffness upon administration to the eye or area surrounding the eye.

One of skill in the art may readily determine the appropriate concentration, or dose, of the LSC of the invention for a particular purpose. The skilled artisan will recognize that a preferred dose is one that produces a therapeutic effect, such as corneal wound healing, in a patient in need thereof. Of course, proper doses of the LSC may require empirical determination at time of use based on several variables, including but not limited to the severity and type of disease, injury, disorder or condition being treated; patient age, weight, sex, health; other medications and treatments being administered to the patient; and the like. A significant amount of LSC is provided to ensure proper treatment. One of skill in the art will also recognize that number of doses (dosing regimen) to be administered needs also to be empirically determined based on, for example, severity and type of disease, injury, disorder or condition being treated. In one embodiment, one dose is sufficient. Other embodiments contemplate, 2, 3, 4, or more doses. In other embodiments, the invention compositions may be coated onto the inner surface of a contact lens which is then placed on the eye, thus allowing delivery of LSC directly to the ocular surface. The LSC used for coating the contact lens may be formulated in a liquid, gel or other suitable ophthalmically compatible vehicle.

Media of the Invention

The invention also provides feeder-free cell culture medium for short term in vitro culture of LSC, LSC-like, SESC or SESC-like comprising a minimum essential medium, a growth factor, a hormone, a soluble factor, and serum or serum substitute. In one embodiment, the shorter term in vitro culture is used to passage about 0 to about passage 4 of LSC, LSC-like, SESC or SESC-like cells.

In an embodiment of the invention, the medium comprises DMEM/F12 medium, DMEM, penicillin-streptomycin, fetal bovine serum, EGF, insulin, hydrocortisone, cholera toxin, and 3,3′,5-triiodo-L-thyronine, wherein the fetal bovine serum may be substituted with human serum or a serum substitute, wherein EGF and insulin may be recombinant EGF and/or recombinant insulin, respectively, preferably recombinant human EGF and recombinant human insulin, and wherein each of the component may be replaced with a functionally equivalent component so long as LSC, LSC-like, SESC or SESC-like cells proliferate and do not differentiate to CEC or skin epidermal cells.

In another embodiment, the medium comprises DMEM/F12 and DMEM (1:1) with fetal bovine serum in the range of about 10-20% or a serum substitute for serum in the range of about 10-20%, EGF in the range of 10-20 ng/ml, insulin in the range of 5-10 μg/ml, hydrocortisone in the range of 0.2-0.8 μg/ml, cholera toxin in the range of 5×10⁻¹¹ to 5×10⁻¹⁰ M and 3,3′,5-triiodo-L-thyronine in the range of 10⁻⁹ M to 4×10⁻⁹ M, wherein EGF and insulin may be recombinant EGF and/or recombinant insulin, respectively, preferably recombinant human EGF and recombinant human insulin, and wherein each of the component may be replaced with a functionally equivalent component so long as LSC, LSC-like, SESC or SESC-like cells proliferate and do not differentiate to CEC or skin epidermal cells.

In yet a further embodiment, the medium comprises DMEM/F12 and DMEM (1:1) with 100 U/ml penicillin, 100 μg/ml streptomycin, 10% fetal bovine serum, 10 ng/ml EGF, 5 μg/ml insulin, 0.4 μg/ml hydrocortisone, 10⁻¹⁰ M cholera toxin and 2×10⁻⁹ M 3,3′,5-triiodo-L-thyronine, wherein the fetal bovine serum may be substituted with a human serum or a serum substitute, wherein EGF and insulin may be recombinant EGF and/or recombinant insulin, respectively, preferably recombinant human EGF and recombinant human insulin, and wherein each of the component may be replaced with a functionally equivalent component so long as LSC, LSC-like. SESC or SESC-like cells proliferate and do not differentiate to CEC or skin epidermal cells.

The invention also provides a feeder-free cell culture medium for long term in vitro culture of LSC, LSC-like, SESC or SESC-like cells comprising a minimum essential medium, a growth factor, a hormone, a soluble factor, serum or serum substitute, and a rho-associated protein kinase (ROCK) inhibitor. In an embodiment of the invention, the long term in vitro culture is used to passage about 17 or more passages of LSC, LSC-like, SESC or SESC-like cells.

In one embodiment, the medium comprises DMEM/F12 medium, DMEM, penicillin-streptomycin, fetal bovine serum, EGF, insulin, hydrocortisone, cholera toxin, and 3,3′,5-triiodo-L-thyronine, and Y-27632, wherein the fetal bovine serum may be substituted with human serum or a serum substitute, wherein EGF and insulin may be recombinant EGF and/or recombinant insulin, respectively, preferably recombinant human EGF and recombinant human insulin, wherein Y-27632 may be replaced with another ROCK inhibitor, and wherein each of the component may be replaced with a functionally equivalent component so long as LSC, LSC-like, SESC or SESC-like cells proliferate and do not differentiate to CEC or skin epidermal cells.

In another embodiment, the medium comprises DMEM/F12 and DMEM (1:1) with fetal bovine serum in the range of about 10-20% or a serum substitute for serum in the range of about 10-20%, EGF in the range of about 10-20 ng/ml, insulin in the range of about 5-10 μg/ml, hydrocortisone in the range of about 0.2-0.8 μg/ml, cholera toxin in the range of about 5×10⁻¹¹ to 5×10⁻¹⁰ M, 3,3′,5-triiodo-L-thyronine in the range of about 10⁻⁹ M to 4×10⁻⁹ M, and Y-27632 in the range of about 1-10 μM, wherein EGF and insulin may be recombinant EGF and/or recombinant insulin, respectively, preferably recombinant human EGF and recombinant human insulin, wherein Y-27632 may be replaced with another ROCK inhibitor, and wherein each of the component may be replaced with a functionally equivalent so long as LSC, LSC-like, SESC or SESC-like cells proliferate and do not differentiate to CEC or skin epidermal cells.

In yet another embodiment, the medium comprises DMEM/F12 and DM EM (1:1) with about 100 U/ml penicillin, about 100 μg/ml streptomycin, about 10% fetal bovine serum, about 10 ng/ml EGF, about 5 μg/ml insulin, about 0.4 μg/ml hydrocortisone, about 10⁻¹⁰ M cholera toxin, about 2×10⁻⁹ M 3,3′,5-triiodo-L-thyronine, and about 1 μM Y-27632 wherein the fetal bovine serum may be substituted with a human serum or a serum substitute, wherein EGF and insulin may be recombinant EGF and/or recombinant insulin, respectively, preferably recombinant human EGF and recombinant human insulin. Y-27632 may be replaced with another ROCK inhibitor. Further, each of the component may be replaced with a functionally equivalent so long as LSC, LSC-like, SESC or SESC-like cells proliferate and do not differentiate to CEC or skin epidermal cells.

The invention additionally provides a feeder-free cell culture medium for long term in vitro culture of LSC or LSC-like cells comprising a minimum essential medium, a growth factor, a hormone, a soluble factor, serum or serum substitute, a rho-associated protein kinase (ROCK) inhibitor and leukemia inhibitory factor (LIF). The long term in vitro culture may be used to passage about 17 or more passages of LSC or LSC-like cells.

In one embodiment, the medium comprises DMEM/F12 medium, DMEM, penicillin-streptomycin, fetal bovine serum, EGF, insulin, hydrocortisone, cholera toxin, and 3,3′,5-triiodo-L-thyronine, Y-27632, and LIF, wherein the fetal bovine serum may be substituted with human serum or a serum substitute, wherein EGF, insulin, and LIF may be recombinant EGF, recombinant insulin, and/or recombinant LIF, respectively, preferably recombinant human EGF, recombinant human insulin, and recombinant human LIF, wherein Y-27632 may be replaced with another ROCK inhibitor, and wherein each of the component may be replaced with a functionally equivalent component so long as LSC or LSC-like cells proliferate and do not differentiate to CECs.

In another embodiment, the medium comprises DMEM/F12 and DMEM (1:1) with fetal bovine serum in the range of about 10-20% or a serum substitute for serum in the range of about 10-20%, EGF in the range of about 10-20 ng/ml, insulin in the range of about 5-10 μg/ml, hydrocortisone in the range of about 0.2-0.8 μg/ml, cholera toxin in the range of about 5×10⁻¹¹ to 5×10⁻¹⁰ M, 3,3′,5-triiodo-L-thyronine in the range of about 10⁻⁹ M to 4×10⁻⁹ M, Y-27632 in the range of about 1-10 μM, and about 5-20 ng/ml LIF. Further, EGF, insulin, and LIF may be recombinant EGF, recombinant insulin, and/or recombinant LIF, respectively. Preferably recombinant human EGF, recombinant human insulin, and recombinant human LIF is used. Further. Y-27632 may be replaced with another ROCK inhibitor. Further still, each of the components may be replaced with a functionally equivalent so long as LSC, LSC-like, SESC or SESC-like cells proliferate and do not differentiate to CECs.

In yet another embodiment, the medium comprises DMEM/F12 and DMEM (1:1) with about 100 U/ml penicillin, 100 μg/ml streptomycin, 10% fetal bovine serum, 10 ng/ml EGF, 5 μg/ml insulin, 0.4 μg/ml hydrocortisone, 10⁻¹⁰ M cholera toxin, 2×10⁻⁹ M 3,3′,5-triiodo-L-thyronine, 1 μM Y-27632 and 10 ng/ml LIF. The fetal bovine serum may be substituted with a human serum or a serum substitute; EGF, insulin, and LIF may be recombinant EGF, recombinant insulin, and/or recombinant LIF, respectively, preferably recombinant human EGF, recombinant human insulin, and recombinant human LIF. Y-27632 may be replaced with another ROCK inhibitor. Additionally, in other embodiments, each of the component may be replaced with a functionally equivalent so long as LSC or LSC-like cells proliferate and do not differentiate to CECs.

Examples of suitable ROCK inhibitors include, but are not limited to, (R)-(+)-trans-4-(1-aminoethyl)-N-(4-Pyridyl)cyclohexanecarboxamide dihydrochloride monohydrate (Y-27632; Sigma-Aldrich), 5-(1,4-diazepan-1-ylsulfonyl) isoquinoline (fasudil or HA 1077; Cayman Chemical), H-1152, H-1152P, (S)-(+)-2-Methyl-1-[(4-methyl-5-isoquinolinyl)sulfonyl]homopiperazine dihydrochloride, Dimethylfasudil (diMF; H-1152P), N-(4-Pyridyl)-N′-(2,4,6-trichlorophenyl)urea, Y-39983, Wf-536, SNJ-1656, and (S)-(+)-2-methyl-1-[(4-methyl-5-isoquinolinyl)sulfonyl]-hexahydro-1H-1,4-diazepine dihydrochloride (H-1152; Tocris Bioscience), imidazole-containing benzodiazepines, imidazopyridine derivative, compound comprising an indazole core, a 2-aminopyridine/pyrimidine core, a 9-deazaguanine derivative, benzamide, or aminofurazan, and derivative and analog thereof, and a combination thereof.

In accordance with the practice of the invention, fetal bovine serum may be substituted with human serum or another serum substitute (depending on the mammalian original of the stem cells so obtained and/or expanded). For example, the stem cells may be from a mammal such as a human, rat, dog, cat, pig, horse, rabbit, cow, monkey or mouse.

In one embodiment, EGF, insulin or LIF is recombinant EGF, recombinant insulin or recombinant LIF, respectively. For example, EGF may be recombinant human EGF. Merely by way of example, insulin may be recombinant human insulin. In another example, LIF may be recombinant human LIF.

In an embodiment, the invention provides a culture medium of the invention having the components of any of the embodiments described above but without an inhibitor of glycogen synthase kinase 3 (GSK3) and/or inhibitor of transforming growth factor β (TGF-β).

In another embodiment, the invention provides a culture medium of the invention consisting essentially of the components of any of the embodiments described above.

In yet another embodiment, hormones as used in the invention include glucocorticoids, hydrocortisone, glucocorticoid receptor agonists, thyroid hormone, 3,3′,5-triiodo-L-thyronine or T3, thyroid receptor agonists, and insulin. Hormones may be synthesized to reflect the structure of the hormones found in nature or may be synthetic/artificial hormones which are not found in nature but are able to modulate the activity of a particular hormone receptor, such as a glucocorticoid receptor, thyroid receptor or insulin receptor.

The following examples are included to demonstrate preferred embodiments of the disclosure. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Materials and Methods

Human Pathology Samples

Corneal epithelium squamous metaplasia and all other tissues were obtained as de-identified surgical specimen, fixed in 5% formalin, embedded into paraffin, sectioned and stained for immunofluorescence studies.

Isolation and Culture of Limbal Stem Cells and Skin Epidermal Stem Cells

Postmortem human eyeballs were obtained from eye banks and limbus region were taken and washed in cold PBS with 100 IU Penicillin and 100 μg/ml Streptomycin, and cut into small pieces. Cell clusters were obtained by 0.2% Collagenase IV digestion at 37° C. for 2 h, single cells were obtained by further digestion with 0.25% Trypsin-EDTA at 37° C. for 15 min. Primary cells were seeded on plastic plates coated with 2% Growth factor reduced Matrigel (354230, BD Biosciences, Inc.). Limbal stem cells from GFP-labeled Rats and Rabbits were isolated and cultured using the same method as for Human LSCs.

Human epidermis was obtained from donor skin biopsy of eye lids, and hair follicles were removed under microscope. Primary human and rabbit epidermal stem cells were isolated from interfollicular epidermis using the same method as described for human limbal stem cells. Culture medium as following: DMEM/F12 and DMEM (1:1) with 1/100 Pen-Strep, 10% fetal bovine serum, 10 ng/ml EGF, 5 μg/ml insulin, 0.4 μg/ml hydrocortisone, 10⁻¹⁰ M cholera toxin and 2×10⁻⁹ M 3,3′,5-triiodo-L-thyronine.

All cells used in this Example 1 are from primary cultured cells made in our labs. The mycoplasma contamination test were routinely done and were negative.

In Vitro 3 Dimensional (3-D) Differentiation Protocol

3-D differentiation was performed on a 24-well plate or an 8-well chamber. Briefly, dissociated single stem cells were embedded in Matrigel® at 2×10⁴ cells/50 μl gel. 3-D structures were formed after 14-18 day culture in a differentiation medium CnT-30 (limbal stem cell differentiation) or CnT-02 (skin epidermal stem cell differentiation) (CelInTec, Inc.).

Immunofluorescence and Laser Confocal Microscopy

To detect the localization of proteins in cultured cells, cells were fixed with 4% paraformaldehyde for 20 min, then permeablized with 0.3% Triton X-100-PBS for 5 min twice and blocked in PBS solution containing 5% bovine serum albumin and 0.3% TritonX-100, followed by an overnight incubation in primary antibodies at 4° C. After 3 washes in PBS, cells were incubated with secondary antibody. Cell nuclei were counterstained with DAPI. For immunofluorescence of paraffin-embedded tissue sections, de-paraffinization was performed, followed by the same immunofluorescence protocol described above.

The following antibodies were used: mouse anti-P63 monoclonal antibody, Rabbit anti-K5 monoclonal antibody, mouse anti-K10 monoclonal antibody, mouse anti-K14 monoclonal antibody with biotin labeled, mouse anti-K19 monoclonal antibody, (MA1-21871, RM2106S0, MS611P0, MS115B0, MS1902P0, Thermo Fisher Scientific, Inc.), Rabbit anti-PAX6 polyclonal antibody (PRB-278P, Covance, Inc.), mouse anti-K1 monoclonal antibody (sc-376224, Santa Cruz, Inc.), Rabbit anti-WNT7A polyclonal antibody, mouse anti-K3/12 monoclonal antibody, Rabbit anti-K12 monoclonal antibody (ab100792, ab68260, ab124975, Abcam, Inc.), mouse anti-Ki67 monoclonal antibody (550609, BD Sciences, Inc), anti-GFP rabbit monoclonal antibody and anti-GFP mouse monoclonal antibody (G10362, A111120, Invitrogen, Inc). The secondary antibodies, Alexa Fluor 488 or 568-conjugated anti-mouse or rabbit immunoglobulin G (IgG) (Invitrogen, Inc.) were used at a dilution of 1:500. Images were obtained using Olympus FV1000 Confocal Microscope.

Real-Time PCR (Q-PCR)

RNA was isolated using an RNeasy kit (Qiagen, Inc.) and subjected to on column DNase digestion, cDNA synthesis was performed using a Superscript III reverse transcriptase kit according to the manufacturer's instructions (Invitrogen, Inc.). Quantitative PCR was performed by 40 cycle amplification using gene specific primers (Table 1) and Power SYBR Green PCR Master Mix on a 7500 Real Time PCR System (Applied Biosystems, Inc.). Measurements were performed in triplicates and normalized to endogenous GAPDH levels. Relative fold change in expression was calculated using the AACT method (CT values <30). Data shown as mean±SD based on three replicates.

TABLE 1
Primer sequences

| Gene | | |
|---|---|---|
| (Human) | Forward Primer | Reverse Primer |
| CASZ1 | GTTCTACGGACAGAAGACCACG | TCTTGAAGCCGTCCTTGGCGTA |
| FGF3 | AGTGGAGCCTGGTCATGGAA | GGATGCTGCCAAACTTGTTCTC |
| FZD5 | TGGAACGCTTCCGCTATCCTGA | GGTCTCGTAGTGGATGTGGTTG |
| GAPDH | GAGTCAACGGATTTGGTCGT | GACAAGCTTCCCGTTCTCAG |
| ID2 | TTGTCAGCCTGCATCACCAGAG | AGCCACACAGTGCTTTGCTGTC |
| K1 | CAGCATCATTGCTGAGGTCAAGG | CATGTCGCCAGCAGTGATCTG |
| K3 | ACGTGACTACCAGGAGCTGATG | ATGCTGACAGACTCGGACACT |
| K5 | GCTGCCTACATGAACAAGGTGG | ATGGAGAGGACCACTGAGGTGT |
| K10 | CCTGCTTCAGATCGACAATGCC | ATCTCCAGGTCAGCTTGGTCA |
| K12 | AGCAGAATCGGAAGGACGCTGA | ACCTCGCTCTTGCTGGACTGAA |
| K14 | TGCCGAGGAATGGTTCTTCACC | GCAGCTCAATCTCCAGGTTCTG |
| K15 | AGGACTGACCTGGAGATGCAGA | TGCGTCCATCTCCACATTGACC |
| K19 | AGCTAGAGGTGAAGATCCGCGA | GCAGGACAATCCTGGAGTTCTC |
| MEIS1 | AAGCAGTTGGCACAAGACACGG | CTGCTCGGTTGGACTGGTCTAT |
| MMP9 | GCCACTACTGTGCTTTGAGTC | CCCTCAGAGAATCGCCAGTACT |
| MMP10 | TCCAGGCTGTATGAAGGAGAGG | GGTAGGCATGAGCCAAACTGTG |
| NR2F2 | TGCACGTTGACTCAGCCGAGTA | AAGCACACTGAGACTTTTCCTGC |
| NOTCH1 | GGTGAACTGCTCTGAGGAGATC | GGATTGCAGTCGTCCACGTTGA |
| NOTCH3 | TACTGGTAGCCACTGTGAGCAG | CAGTTATCACCATTGTAGCCAGG |
| ODZ3 | GGACAAGGCTATCACAGTGGAC | TTCTGAGGGAGCCGTCATAACC |
| PAX6 | TGTCCAACGGATGTGTGAGT | TTTCCCAAGCAAAGATGGAC |
| PDGFA | CAGCGACTCCTGGAGATAGACT | CGATGCTTCTCTTCCTCCGAATG |
| PPARG | AGCCTGCGAAAGCCTTTTGGTG | GGCTTCACATTCAGCAAACCTGG |
| PRD38 | CTGTGTCCTGAGCCATACTTCC | CCTTCTGAGGAACCATTTGCTC |
| TGFB1 | AGGACTGACGGAGACCCTCAAC | TCCGCTAACCAGGATTTCATCAC |
| WNT7A | TGCCCGGACTCTCATGAAC | GTGTGGTCCAGCACGTCTTG |
| Gene | | |
| (Rabbit) | Forward Primer | Reverse Primer |
| GAPDH | GCGAGATCCCGCCAACATCAAGT | AGGATGCGTTGCTGACAATC |
| PAX6 | GTATTCTTGCTTCAGGTAGAT | GAGGCTCAAATGCGACTTCAGCT |

Primers used for PAX6 transuction

| | |
|---|---|
| PAX6 | TTCCCGAATTCTGCAGACCCATGCAGATGCAAAAGTCCAAGTGCTGGACAA |
| Inf | TCAAAACGTGTCCAACGGATGTG |
| PAX6 | CACATCCGTTGGACACGTTTTGATTGTCCAGCACTTGGACTTTTGCATCTGC |
| InR | ATGGGTCTGCAGAATCGGGAA |

Genome Wide Gene Expression Microarray and Data Analysis

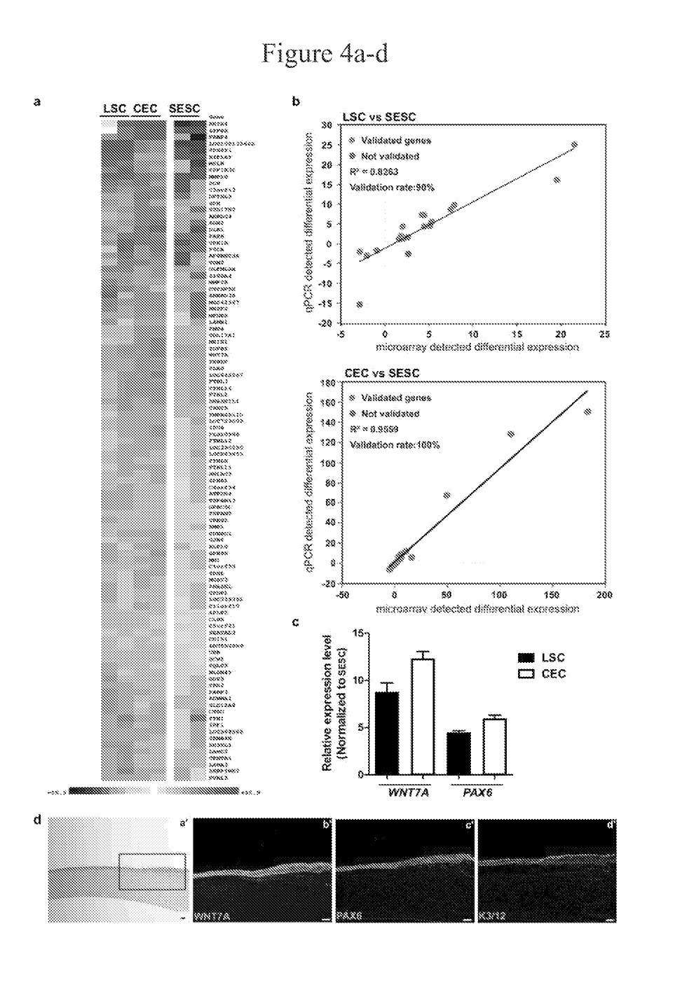

Total RNA was isolated from LSCs, SESCs and differentiated CECs from 3-D differentiation assay. Gene expression microarray analysis was performed using an Illumina human genome microarray system, with each sample in biological replicate (n=2 per group; Human HT-12 v4 Expression BeadChip; Illumina, San Diego, Calif.). Raw data was deposited into the GEO database under accession number GSE32145. Expression level data were generated by the Illumina BeadStudio version 3.4.0 and normalized using quartile normalization. Probes whose expression level exceeded a threshold value of 64 in at least one sample were considered detected. The threshold value was found by inspection from the distribution plots of log 2 expression levels. Detected probes were sorted according to their q-value, which is the smallest false discovery rate (FDR) at which the probe is called significant. FDR was evaluated using Significance Analysis of Microarrays and its implementation in the official statistical package sam¹⁹. To avoid false positive calls due to spuriously small variances, the percentile of standard deviation values used for the exchangeability factor s0 in the regularized t-statistic was set to 50. We combined the LESCs and CECs samples into one group of four samples, and looked for differentially expressed genes between this group and SESCs samples. The top 100 significant genes in this comparison are presented in FIG. 4. All genes in this figure are significant at the FDR level of 0.01 or less. A heatmap was created using in-house hierarchical clustering software, and colors qualitatively correspond to fold changes.

RNA-Seq and Hierarchical Cluster Analysis

Total RNA was purified by a Picropure RNA isolation kit (Life Technology). RNA-seq was performed as previous described²⁰. Briefly, 600 ng of total RNA was first converted to cDNA by superscript III first strand synthesis kit with primer Biotin-B-T. The cDNA was purified by NucleoSpin Gel and PCR Clean-Up Kit column (Clontech) to remove free primers and enzyme. Then, terminal transferase (NEB) was applied to block the terminal of a cDNA 3′ end. Streptavidin-coaged magnetic beads (Life Technology) were further applied to isolate cDNAs. After RNA degradation by sodium hydroxide, second strand cDNA was synthesized by random priming with primer A-N8. The second strand cDNA was eluted from beads by heat denaturing. The cDNA was then used as template to construct libraries by amplifies with barcode primers and primer PB. The sequencing was done on Hiseq 2000 system.

Hierarchical cluster analysis was performed with cluster and Java TreeView²¹. The raw data was first filtered using default parameters provided by Cluster program, the filtered data was further adjusted by log transformation, centered gene and array with median, and then hierarchically cluster both gene and array with Euclidean and average linkage. The hierarchical trees and gene matrix was visualized and generated by Java Treeview.

Lentiviral RNAi and PAX6 Transduction

Lentiviral shRNAs targeting PAX6, WNT7A, and FZD5 genes were either cloned into pLKO.1 plasmid between Age I and EcoR I or directly purchased from Sigma. ShRNAs-targeting sequences for gene specific knockdowns were as follows: PAX6, CGTCCATCTTTGCTTGGGAAA and AGTTTGAGAGAACCCATTATC; WNT7A, CGTGCTCAAGGACAAGTACAA and GCGTTCACCTACGCCATCATT; FZD5, CGCGAGCCCTTCGTGCCCATT and TCCTAAGGTTGGCGTTGTAAT. We used a lentiviral pLKO.1-puro Non-Target shRNA control plasmid encoding a shRNA that did not target any known genes from any species as a negative control in all gene knockdown experiments (Sigma, Inc.).

Lentiviral shRNA particles were prepared according to a previous described protocol²². Briefly, Replication-incompetent lentiviral particles were packaged in 293T cells by co-transfection of shRNA constructs with packaging mix (pCMV-dR8.2 and pCMV-VSVG at 9:1 ratio). Virus was harvest two times at 48 hrs and 72 hrs post-transfection.

For transduction, PAX6a ORF was PCR amplified from cDNAs purchased from Thermo Scientific (MHS6278-202756612) and inserted into pLenti CMV-GFP Puro vector between BamH1 and BsrG1. PAX6b was generated by PCR mediated point mutation strategy with primers PAX6 InF and PAX6 InR (Table 1). For GFP-labeling, pLenti CMV-GFP Hygro (656-4) purchased from Addgene was used. The lentiviral particles were packaged by co-transfection with packaging plasmids psPax2 and pMD2.G.

For lentiviral infection, cells were infected for 16-20 hrs with fresh media containing individual virus and polybrene at a final concentration of 8 μg/ml. The infected cells were further selected by 2 μg/ml puromycin for 48 hrs or 200 μg/ml hygromycin for 72 hrs.

Western Blot and Co-Immunoprecipitation (Co-IP)

For western blotting, cells were washed once with PBS and then collected in cell lysis buffer (50 mM Tris-HCl, pH6.8; 2% SDS; 10% glycerol; 100 mM DTT). Protein concentration was quantified by Nanodrop and Bromophenol blue was added to a final concentration of 0.1%, then 25 μg of total lysate was fractionated on a 4-12% NUPAGE gel (Life Technology, Inc). Proteins were transferred to a nitrocellulose membrane at 100V for 1 hour. The membrane was blocked with 5% milk and probed with relevant antibodies and mouse anti-3-actin monoclonal antibody (A5316, Sigma, Inc.).

To detect interaction between FZD5 and WNT7A, a 10-cm dish of limbal stem cells at 90% confluence was collected; the cell pellet was resuspended in 7001 μl of Co-IP buffer (10 mM Tris-HCl, pH 7.4, 100 mM NaCl, 2.5 mM MgCl₂, 0.5% NP-40, 1× proteinase inhibitor) and incubated on ice for 20 minutes, then centrifuge at 13,000 rpm at 4° C. for 20 minutes. The 600 μl of supernatant was aliquot into two pre-chilled eppendorf tubes, 5 μg of rabbit anti-FZD5 monoclonal antibody (#5266, Cell signaling, Inc.) or WNT7A antibodies was added to each tube and incubated at 4° C. overnight. 50 μl of protein A/G magnetic beads (Thermo Fisher, Inc.) were added to each tube, and incubated at 4° C. for two hours, washed with a Co-IP buffer and eluted in 1×SDS sample buffer (Life Technology, Inc.) at 70° C. The input and elutes were fractionated on 4-12% NUPAGE gel and blotted with FZD5 and WNT7A antibodies.

Cell Transplantation

All animal studies were performed in full accordance with the ARVO statement for the Use of Animals in Ophthalmic and Vision Research and approval of Institutional Animal Care Committees were obtained.

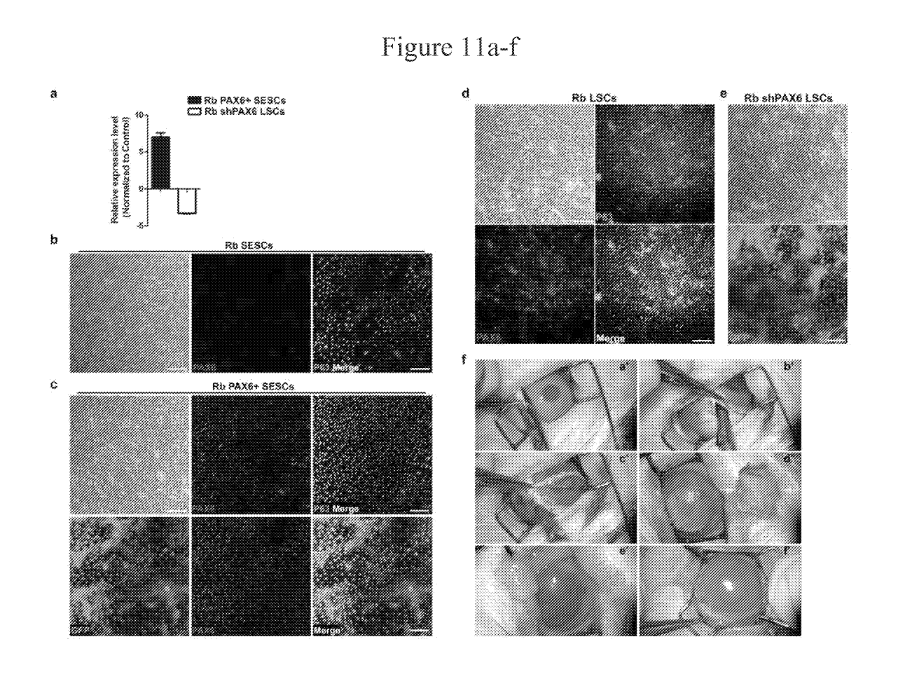

New Zealand white rabbits (2.0 kg-2.5 kg, male) were used in the study. Rabbits were anesthetized with intramuscular injection of xylazine hydrochloride (2.5 mg/mL) and ketamine hydrochloride (37.5 mg/mL). To create a limbal stem cells deficiency model (FIG. 11f), corneal and limbal epithelium was removed by 360 degree conjuntival peritomy and lamellar dissection to remove anterior scleral and corneal stromal tissues, 2 mm posterior from limbus towards the center of the cornea. This dissection ensured removal of LSC and the entire corneal epithelium. 5×10⁵ rabbit GFP-labeled LSCs, PAX6+ SESCs or shPAX6 LSCs cells were mixed with fibrin (25 mg/ml) and thrombin (25 U/ml) and seeded onto the exposed stromal bed of a recipient cornea and limbal area; the surface was then covered by a human amniotic membrane (Bio-tissue, Inc. USA) which is secured with 10.0 VICRYL sutures (ETHICON, USA) (Table 2).

TABLE 2
Summary of rabbit transplantation results

Rabbit number

| | | | Died from systemic |
|---|---|---|---|
| GFP-labeled | Regeneration | Opaque and | infection |
| donor | and re- | vascularized | or unrelated |
| cells | epithelization | corneal surface | complications |
| LSCs | 3 | 0 | 0 |
| PAX6 + SESCs | 5 | 0 | 2 |
| shPAX6 LSCs | 0 | 4 | 1 |

As a negative control, only amniotic membrane was applied to the denuded cornea. Antibiotics (levofloxacin) and steroids (betamethasone) were applied to both eyes immediately after the cell transplant procedures, and was administered three times a day for 2 weeks. Animals were randomly assigned into each experimental group. The investigator who performed cell transplantation was blinded by the identity of cells used. Another investigator was used for assessment of effect of corneal epithelial repair in rabbits and was blinded by the identity of cells used in the transplantation. For analysis, we only exclude animals that died of post-operative complications such as infection, as they did not reach an end point for assessment of effect of cell transplantation; this criteria is pre-established.

Results and Discussion

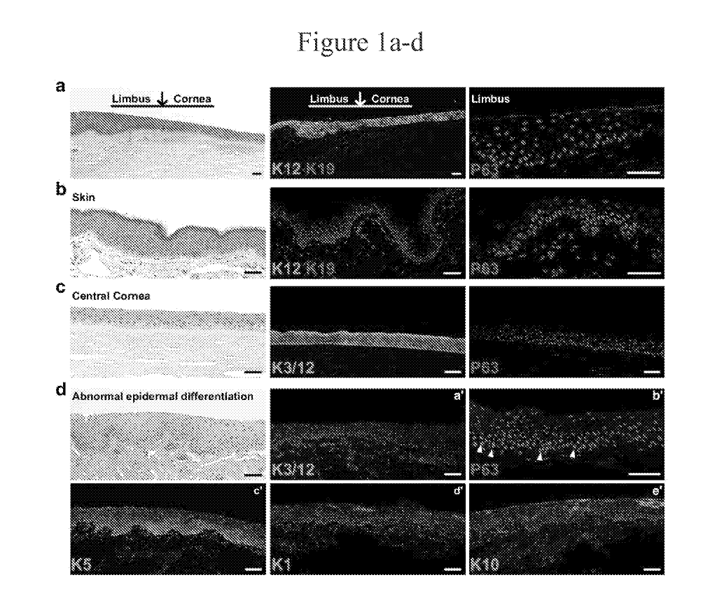

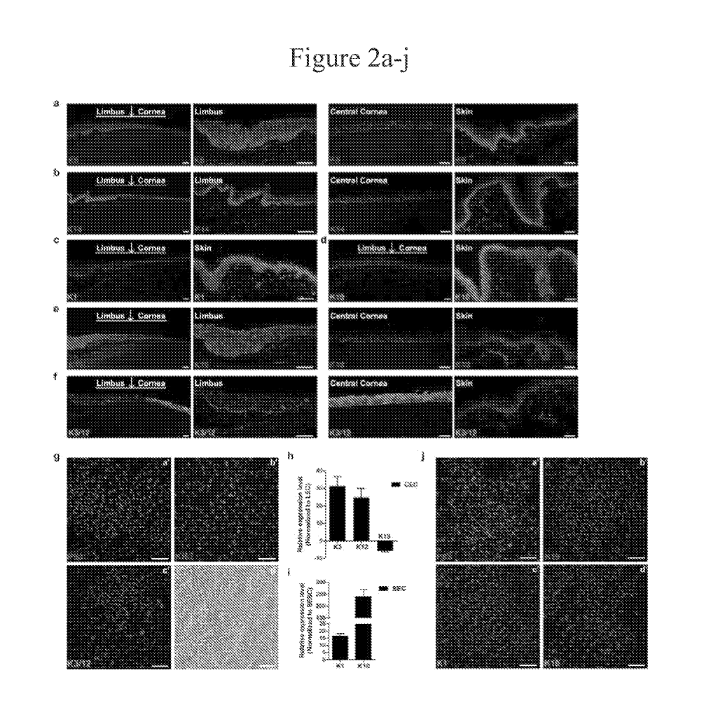

Corneal and skin epithelium share many similarities, including a typical morphology of stratified epithelium and maintenance of their stem cells by p63 in the Keratin5/Keratin14+ (K5/K14) basal cell layer in limbus and epidermis⁴⁻⁸ (FIGS. 1a, 1b, 2a and 2b). However, there are marked differences between them. Skin epithelial stem cells (SESCs) move upward from a deep to suprabasal layers vertically during differentiation^{9,10}, where K5 and K14 are replaced by skin specific K1/K10 (ref. 11; FIGS. 2c and 2d). In contrast, LSCs (defined by K19 at the limbus¹², see FIGS. 1a and 2e) migrate centripetally for several millimeters to the central cornea during which it undergoes differentiation and K5/K14 are replaced by corneal specific K3 and K12 (ref. 13, 14; FIGS. 1c and 2f).

A clear, transparent cornea maintained by CECs is essential for vision. Pathological conversion of CECs into skin-like epithelial cells, as indicated by morphological changes and switches in keratin expression (e.g. replacement of corneal specific K3/K12 by skin specific K1/K10 along with K5 positive cells at the basal layer, see FIG. 1d), leads to the loss of transparency in the cornea and causes millions of people around the world to suffer from partial or complete blindness³, but the underlying mechanism has remained largely unknown.

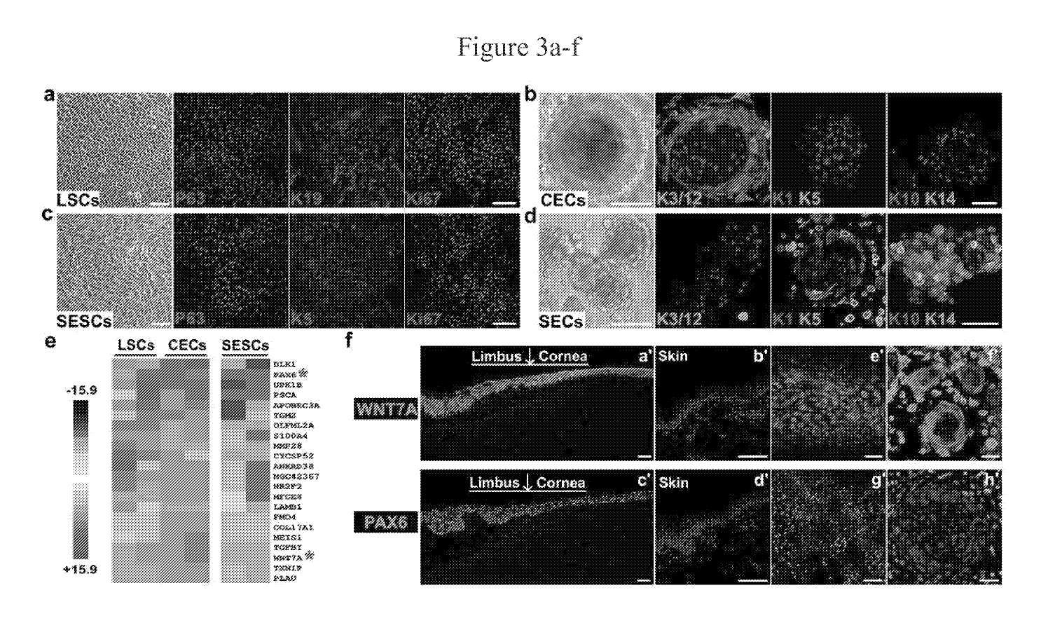

To elucidate potential disease mechanisms, we successfully developed a feeder-free cell culture protocol to expand LSCs from human donors, enabling us to generate a homogeneous cell population to delineate key factors involved in controlling LSC cell fate determination and CEC differentiation. Proliferating LSCs were characterized by positive p63 and K19 with a high percentage of mitotic marker Ki67 (FIGS. 2g and 3a). We next established a 3 dimensional (3-D) LSC differentiation protocol to establish a 3-D CEC sphere structure from a single LSC within 14-18 days, as evidenced by strong expression of the CEC specific markers K3/K12 (FIG. 3b). The 3-D differentiation sphere was further characterized by key differences in gene expression between LSCs and CECs, the latter of which showed increased expression of K3 (↑31.2 fold) and K12 (↑24.7 fold) and concomitant decreased expression of K19 (↓6.2×, all p<0.01; see FIG. 2h). We took a similar strategy to expand SESCs and observed strong expression of typical SESC markers P63 and K5 in cultured SESCs (FIG. 3c). As expected, we detected increased expression of epidermal differentiation markers K1 (↑16.6 fold) and K10 (↑225.8 fold) in 3-D differentiated skin epithelial cells (SECs) compared to SESCs (FIGS. 2i, 2j and 3d).

To identify additional genes uniquely expressed in LSCs, CECs and SESCs, we performed genome-wide gene expression analysis (FIGS. 3e, 4a and 4b). Among genes that were differentially expressed, we focused on signaling molecules and transcription factors because of their central roles in cell fate determination and differentiation. We identified WNT7A and PAX6 that were highly expressed in LSCs and CECs when compared to SESCs (PAX6, ↑8.8 fold in LSCs and ↑12.3 fold in CECs, p<0.001; WNT7A, ↑4.5 fold in LSCs, ↑6.0 fold in CECs, p<0.001) (FIGS. 3e and 4c). We observed that WNT7A expression precisely mirrored the expression pattern of PAX6 in in vitro LSC and CEC cultures, and in in-vivo epithelial layers of cornea and limbus from infant to adult, while both of these genes were undetectable in skin epidermis (FIGS. 3f and 4d).

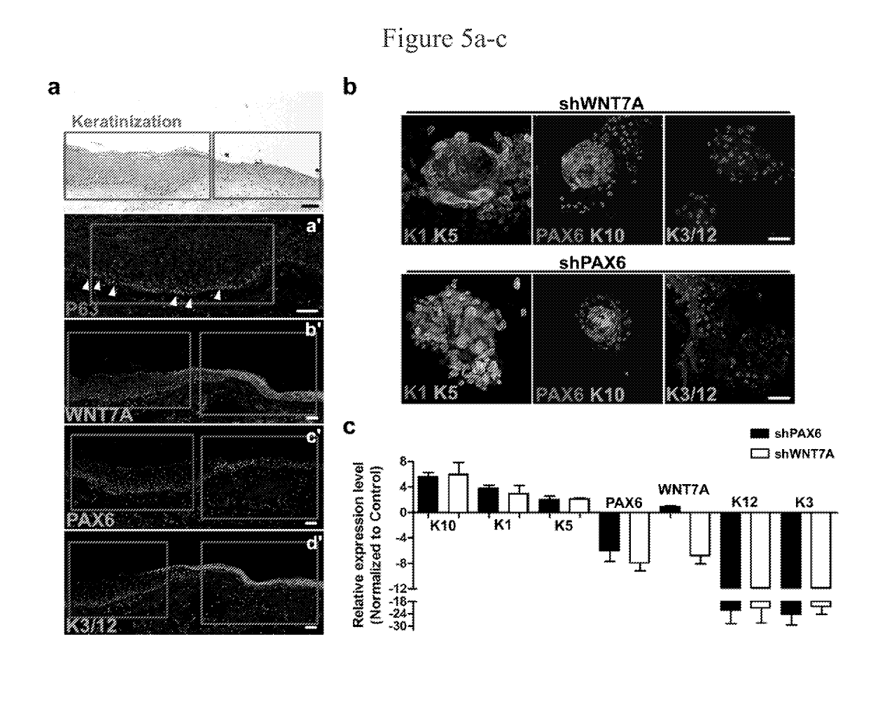

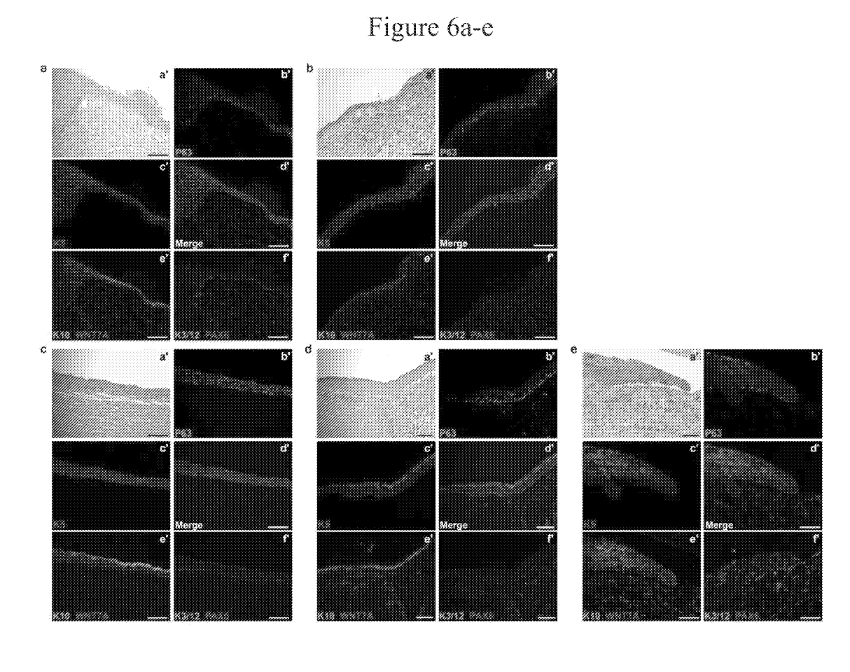

To determine the clinical relevance of WNT7A and PAX6 expression in LSCs and CECs, we examined several types of human corneal diseases, corneal epithelium squamous metaplasia, inflammatory keratopathy, trauma and alkaline burn. We observed the localized expression of p63 and K5 at the basal layer (FIGS. 5a and 6), and the expression of K10 in the suprabasal layer (FIGS. 1d and 6). We also found that WNT7A/PAX6 and K3/12 expression were conspicuously absent in areas of metaplasia, while they were positive in surrounding corneal epithelium (FIGS. 5a and 6).

These results suggest cornea epithelial cells were switched to skin-like epithelial cells in patient tissues with these disease conditions.

Wnt molecules are secreted signaling proteins that play a critical role in controlling cell fate decisions and tissue specification¹⁵. PAX6 is also a well-known control gene for eye development and disease¹⁶. However, it has remained unclear whether the loss of PAX6 is the cause or the consequence of abnormal skin epidermal differentiation in ocular surface diseases.

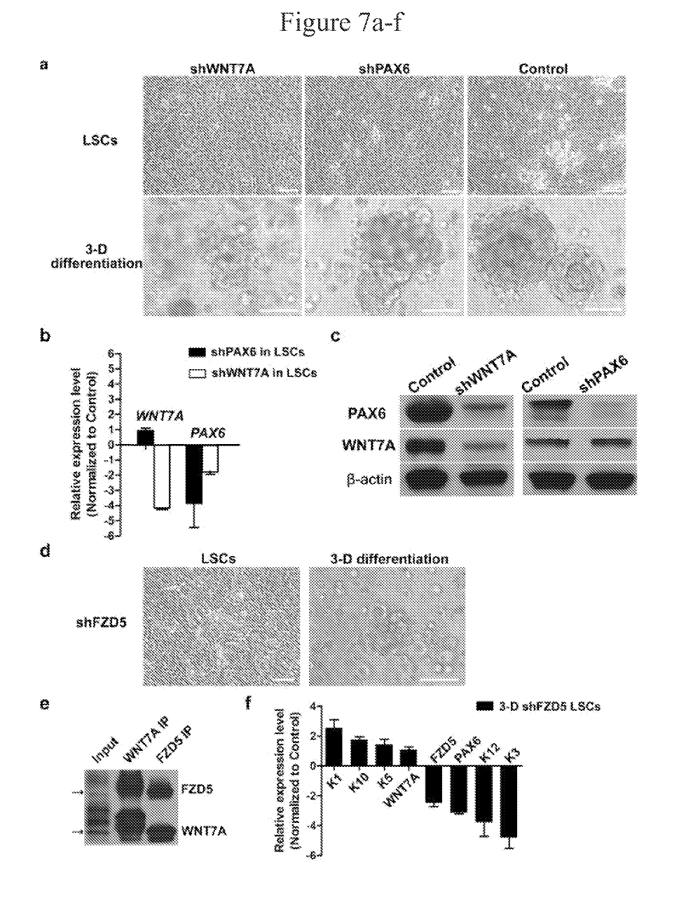

To demonstrate WNT7A and PAX6 are necessary for LSC and CEC cell fate determination and differentiation, we used lentiviral shRNAs to specifically knock them down in LSCs. While LSCs with knockdown of either WNT7A or PAX6 did not change proliferation and morphological properties (FIG. 7a), these treatments significantly diminished the expression of corneal K3/K2 under the 3-D differentiation conditions (WNT7A knockdown: ↓24.7 fold in K3, ↓22.6 fold in K12; PAX6 knockdown: ↓20.8 fold in K3, ↓21.4 fold in K12; all P<0.05), and concurrently, the expression of skin-specific K1/K10 became more prominent (WNT7A knockdown: ↑3.9 fold in K1 and ↑5.7 fold in K10; PAX6 knockdown: ↑3.1 fold K1 and ↑6.1 fold in K10; all P<0.05), indicative of more skin-like differentiation (FIGS. 5b and 5c). Moreover, knockdown of WNT7A reduced PAX6 expression in LSCs (↓1.8 fold, p<0.001); this repressive effect was even stronger in differentiated CECs (↓8.0 fold, p<0.01). In contrast, there was no significant difference in WNT7A expression when PAX6 was knocked down in either LSCs or CECs (FIGS. 5c, 7b and 7c). These results suggest that WNT7A acts upstream of PAX6 during CEC differentiation.

To further study the role of the Wnt signaling pathway in corneal fate determination and differentiation, we investigated the functional requirement of Frizzled receptors (FZD), which has been shown to interact and transduce WNT7A signaling based on co-immunoprecipitation¹⁷. We found that WNT7A interacted strongly with FZD5 in LSCs (FIGS. 7d and 7e), and as predicted, knockdown of FZD5 in LSCs also led to reduced PAX6 expression (↓1.7 fold in LSCs and ↓3.0 fold in differentiated CECs (p<0.001) (FIG. 7f). Together, these data demonstrated that loss of WNT7A or PAX6 led to a switch of corneal epithelial cells to skin-like epidermal cells and that WNT7A/FZD5 acted as the upstream regulators of PAX6 expression in corneal differentiation.

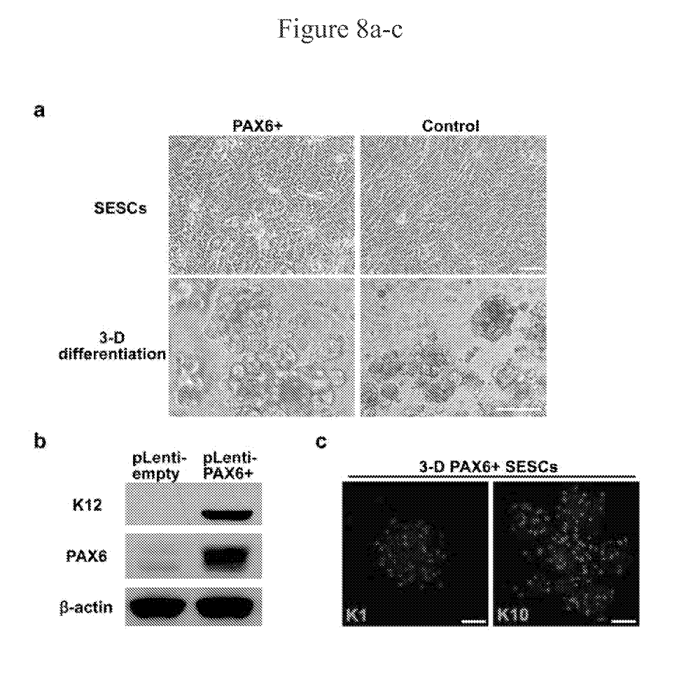

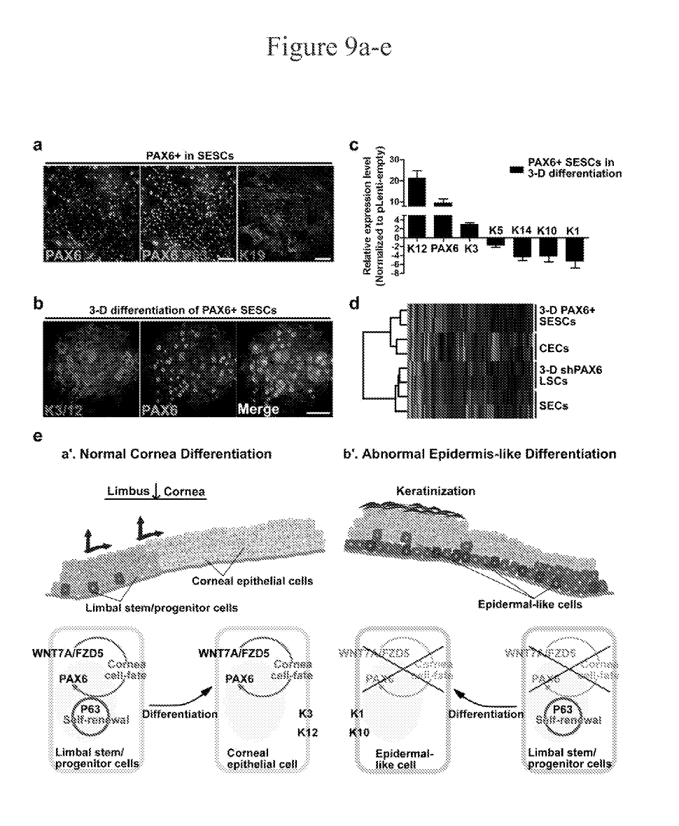

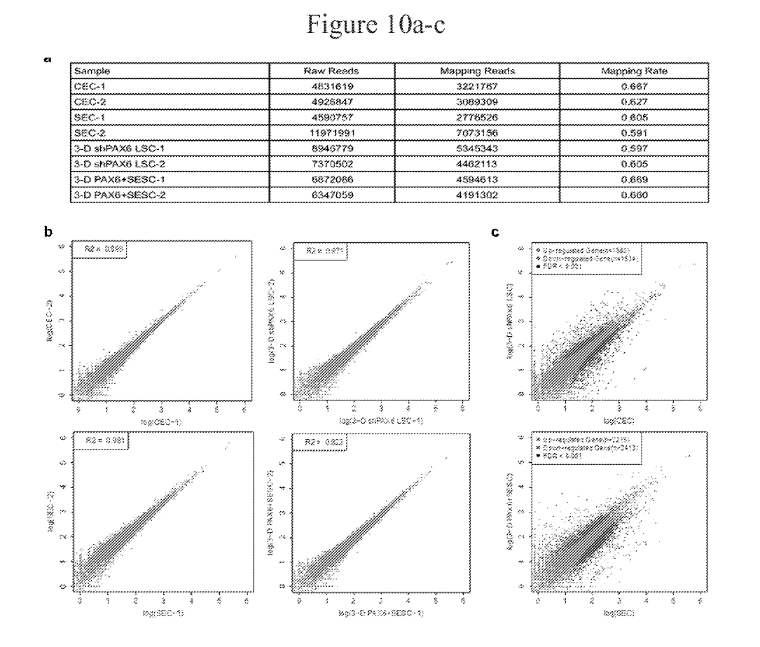

Given the central role of PAX6 in eye development¹⁶, we next tested the possibility that engineered expression of PAX6 might be able to convert SESCs into LSC-like cells (FIG. 8a). Indeed, we found that the expression of either PAX6a or PAX6b in SESCs was sufficient to convert them into LSC-like cells, as evidenced by the induced K19 expression on the surface, coincident with the expression of both P63 and PAX6 in the nucleus (FIG. 9a). When placed in 3-D culture, PAX6-transduced SESCs showed dramatic increase in corneal K3/K12 expression (↑9.4 fold and ↑72.7 fold, all p<0.05) with concomitant decrease in skin K1/K10 expression (↓20.8 fold and ↓20.0 fold, all p<0.01) (FIGS. 8b, 8c, 9b and 9c). To obtain global evidence for successful cell fate conversion, we performed gene expression profiling by RNA-seq¹⁸ on CECs, SECs, and LSCs after knocking down PAX6 and on SESCs transduced with PAX6 upon 3-D differentiation. We generated 3 to 7 million reads from each biological sample that were uniquely mapped to the Refseq database (FIG. 10a). Pair-wise comparison demonstrated that the data were very reproducible within the same group of samples (FIG. 10b), in contrast, when compared between cells with different fates, the data demonstrate remarkable differences based on the statistical cut-off of FDR<0.001 (FIG. 10c). We displayed the entire datasets that recorded the expression of >10,000 genes in various cell types (FIG. 9d), demonstrating that both induced (red) and repressed (green) genes were clearly co-segregated between CECs and PAX6+ SESCs and between shPAX6-treated LSCs and SECs. These data therefore provided global evidence for a role of the WNT7A/PAX6 axis in cell fate conversion from SESCs to CECs. Together, these data suggest defects in the WNT7A/PAX6 axis are likely responsible for metaplastic conversion of corneal cells to skin epidermal-like cells in corneal diseases in humans (illustrated in FIG. 9e), although further studies need to be performed to determine the significance of the WNT7 and PAX6 axis in corneal epithelial differentiation.

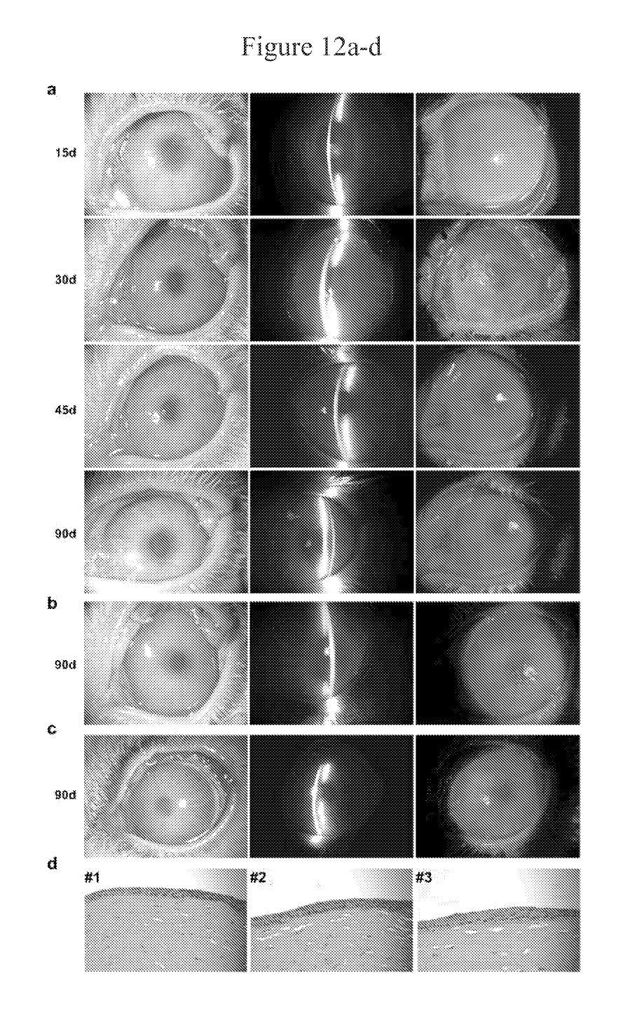

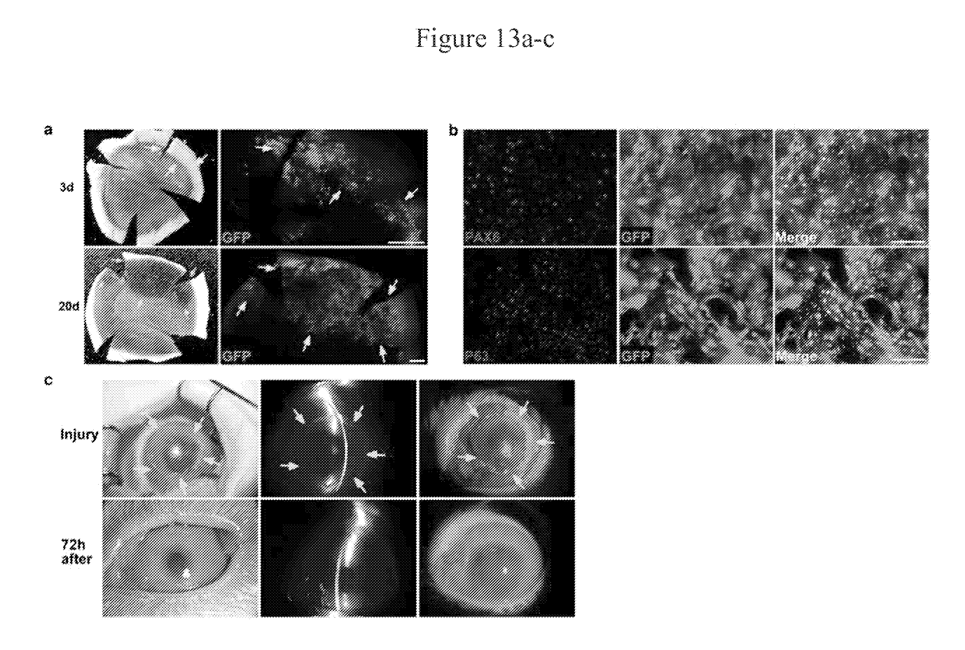

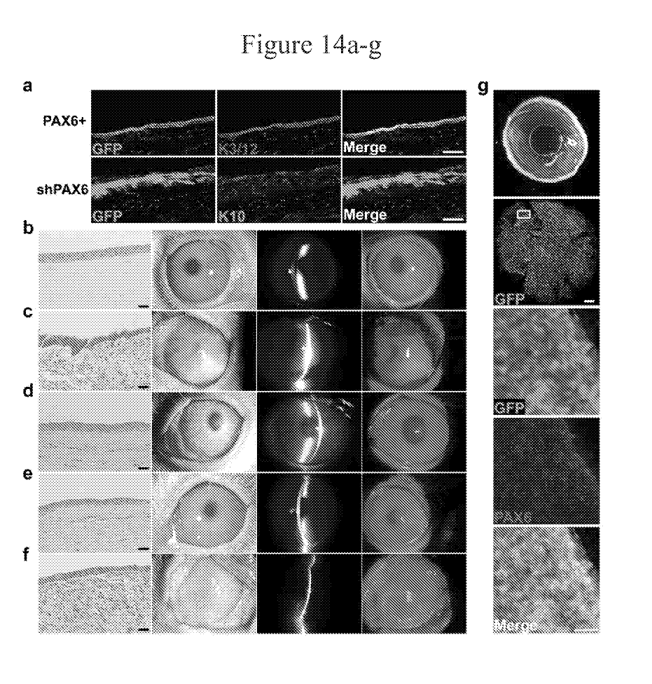

Finally, we tested the potential that SESCs with engineered expression of PAX6 (FIGS. 11a, 11 b and 11c) might be used to treat and repair corneal epithelial defects on a rabbit LSC deficiency model (FIG. 11f), which mimics a common corneal disease condition in humans. We showed that rabbit SESCs with PAX6 transduction formed a continuous sheet of epithelial cells with positive staining of corneal specific K3/12 (FIG. 14a) and successfully repaired epithelium defect of the entire corneal surface to restore and maintain normal cornea clarity and transparency for over 3 months (FIGS. 14b-14g and 12). By following the time course of corneal epithelial surface repair by using GFP-labeled PAX6+ SESCs, we observed that these PAX6-reprogrammed SESCs were initially only located at the limbal region and then moved progressively towards the central cornea with corresponding areas of restored cornea clarity (FIG. 13a). Importantly these grafted cells were indeed able to repopulate limbus as evidenced by culture and re-isolation of PAX6+ SESCs from limbal region (FIG. 13b). Strikingly, these reprogrammed SESCs were capable of repairing large corneal epithelium defects after repeated corneal epithelial scraping (FIG. 13c). In sharp contrast, transplanting rabbit LSCs with PAX6 knockdown (FIGS. 11a, 11d and 11e) onto denuded corneal surface resulted in a K10 positive skin-like epithelium with opacity and vascularization (FIG. 14f). Together, these data demonstrate that SESCs with PAX6 expression are able to transdifferentiate into corneal-like epithelium and repair corneal surface defects.

In summary, this work establishes the feasibility of expanding LSCs under feeder-free conditions and its therapeutic potential, and demonstrates key roles of WNT7A and PAX6 in corneal lineage specification. Importantly, SESCs or other cell types converted into a corneal fate by PAX6 expression may serve as a potential source for corneal surface repair and regeneration, particularly in patients with total LSC deficiency. This would overcome a major feasibility problem in using patent's own LSCs for transplantation, thus pointing to a potential therapeutic strategy for treating many common corneal diseases in humans.

### REFERENCES

1 Davanger, M. & Evensen, A. Role of the pericorneal papillary structure in renewal of corneal epithelium. Nature 229, 560-561 (1971).
2 Cotsarelis, G., Cheng, S. Z., Dong, G., Sun, T. T. & Lavker, R. M. Existence of slow-cycling limbal epithelial basal cells that can be preferentially stimulated to proliferate: implications on epithelial stem cells. Cell 57, 201-209 (1989).
3 Li, W. et al. Down-regulation of Pax6 is associated with abnormal differentiation of corneal epithelial cells in severe ocular surface diseases. The Journal of pathology 214, 114-122, doi:10.1002/path.2256 (2008).
4 Pellegrini, G. et al. p63 identifies keratinocyte stem cells. Proceedings of the National Academy of Sciences of the United States of America 98, 3156-3161, doi: 10.1073/pnas.061032098 (2001).
5 Mills, A. A. et al. p63 is a p53 homologue required for limb and epidermal morphogenesis. Nature 398, 708-713, doi: 10.1038/19531 (1999).
6 Yang, A. et al. p63 is essential for regenerative proliferation in limb, craniofacial and epithelial development. Nature 398. 714-718, doi:10.1038/19539 (1999).
7 Koster, M. I., Kim. S., Mills, A. A., DeMayo, F. J. & Roop, D. R. p63 is the molecular switch for initiation of an epithelial stratification program. Genes & development 18, 126-131, doi:10.1101/gad.1165104 (2004).
8 Rama, P. et al. Limbal stem-cell therapy and long-term corneal regeneration. The New England journal of medicine 363, 147-155, doi: 10.1056/NEJMoa0905955 (2010).
9 Blanpain, C. & Fuchs, E. Epidermal homeostasis: a balancing act of stem cells in the skin. Nature reviews. Molecular cell biolog 10, 207-217, doi:10.1038/nrm2636 (2009).
10 Arwert, E. N., Hoste, E. & Watt, F. M. Epithelial stem cells, wound healing and cancer. Nature reviews. Cancer 12, 170-180, doi: 10. 1038/nrc3217 (2012).
11 Kopan, R. & Fuchs, E. A new look into an old problem: keratins as tools to investigate determination, morphogenesis, and differentiation in skin. Genes & development 3, 1-15 (1989).
12 Lauweryns, B., van den Oord, J. J. & Missotten, L. The transitional zone between limbus and peripheral cornea. An immunohistochemical study. Investigative ophthalmology & visual science 34, 1991-1999 (1993).
13 Eichner, R., Bonitz, P. & Sun, T. T. Classification of epidermal keratins according to their immunoreactivity, isoelectric point, and mode of expression. The Journal of cell biology 98, 1388-1396 (1984).
14 Schlotzer-Schrehardt, U. & Kruse, F. E. Identification and characterization of limbal stem cells. Experimental eye research 81, 247-264, doi: 10.1016/j.exer.2005.02.016 (2005).
15 Dorsky, R. I., Moon, R. T. & Raible, D. W. Control of neural crest cell fate by the Wnt signalling pathway. Nature 396, 370-373, doi: 10.1038/24620 (1998).
16 Eiraku, M. et al. Self-organizing optic-cup morphogenesis in three-dimensional culture. Nature 472, 51-56. doi:10.1038/nature09941 (2011).
17 von Maltzahn, J., Bentzinger, C. F. & Rudnicki, M. A. Wnt7a-Fzd7 signalling directly activates the Akt/mTOR anabolic growth pathway in skeletal muscle. Nature cell biology 14, 186-191, doi:10.1038/ncb2404 (2012).
18 Yu, F-X, Zhao, B., Panupinthu, N., Jewell, J. L., Lian, I., Wang, L. H., Zhao, J., Yuan, H., Tumaneng, K., Li, H., Fu, X-D, Mills, G. B., Guan, K-L (2012). Regulation of the Hippo-YAP pathway by G-protein-coupled receptor signaling. Cell, 150:780-791.
19 Tusher, V. G., Tibshirani, R. & Chu, G. Significance analysis of microarrays applied to the ionizing radiation response. Proceedings of the National Academy of Sciences of the United States of America 98, 5116-5121, doi: 10.1073pnas.091062498 (2001).
20 Fox-Walsh, K., Davis-Turak, J., Zhou, Y., Li, H. & Fu, X. D. A multiplex RNA-seq strategy to profile poly(A+) RNA: application to analysis of transcription response and 3′ end formation. Genomics 98, 266-271, doi:S0888-7543(11)00083-8 [pii] 10.1016/j.ygeno.2011.04.003.
21 Eisen, M. B., Spellman, P. T., Brown, P. O. & Botstein, D. Cluster analysis and display of genome-wide expression patterns. Proc Natl Acad Sci USA 95, 14863-14868 (1998).
22 Xue, Y. et al. Direct conversion of fibroblasts to neurons by reprogramming PTB-regulated microRNA circuits. Cell 152, 82-96, doi:S0092-8674(12)01433-X [pii]10.1016/j.cell.2012.11.045.

### Example 2

Materials and Methods

Animals

ROSA^{mT/mG} a mice were described previously (PMID: 17868096; 28) and maintained as homozygotes. The P0-3.9-GFPCre mice, expressed an EGFP-Cre recombinase fusion protein under the control of the Pax6 P0 enhancer, were maintained on a FVB background and PCR genotyped as described (29).

Lineage Tracing

Lineage tracing experiments were performed by crossing homozygous GFP reporter mice (ROSA^{mT/mG}) with lens specific Cre transgenic mice (P0-3.9-GFPCre) in which Cre expression is under the control of mouse Pax6 ectoderm enhancer. Eyes were dissected at postnatal day (P) 1 and P60 and fixed in 4% formaldehyde overnight. Tissues were then incubated in 10% sucrose and embedded in optimal cutting temperature medium for cryosectioning. Frozen sections were washed in PBS and imaged on a Zeiss Axio Imager fluorescence microscope.

Isolation and Culture of Human LSCs and Skin Epidermal Stem Cells (SESCs)

Postmortem human eyeballs were obtained from eye banks; human epidermis was obtained from donor skin biopsy of eyelids. Limbus regions were excised and washed in cold PBS with 100 IU/ml penicillin and 100 μg/ml streptomycin. After limbus regions were cut into small pieces, cell clusters were obtained by 0.2% collagenase IV digestion at 37° C. for 2 h. This was followed by further digestion with 0.25% trypsin/EDTA at 37° C. for 15 min to obtain single cells. Primary cells were seeded on plastic plates coated with 2% growth factor-reduced Matrigel® (354230, BD Biosciences, Inc.). Primary human SESCs were isolated from interfollicular epidermis using the same treatment.

Culture medium for both limbal stem cells (LSCs) and skin epidermal stem cells (SESCs) contained DMEM/nutrient mixture F-12 and DMEM (1:1) with 100 IU/ml penicillin, 100 IU/ml streptomycin, 10% fetal bovine serum, 10 ng/ml epidermal growth factor (EGF), 5 μg/ml insulin, 0.4 μg/ml hydrocortisone, 10⁻¹⁰ M cholera toxin, and 2×10⁻⁹ M 3,3′,5-triiodo-L-thyronine. To differentiate LSCs, cells were cultured in CnT-30 (CelIntec, Inc.) for 8-12 days.

Real-Time Quantitative PCR (qPCR)

An RNeasy kit (Qiagen, Inc.) was used to isolate RNA. On-column DNase digestion was performed. A Superscript III reverse transcriptase kit (Invitrogen, Inc.) was used for cDNA synthesis according to the manufacturer's instructions. A real-time PCR system (Applied Biosystems, Inc.) was used to perform quantitative PCR. Forty cycles of amplification were carried out using gene specific primers (Table 3) and Power SYBR Green PCR Master Mix. Measurements were performed in triplicates and normalized to endogenous GAPDH levels. Relative fold change in expression was calculated using the ΔΔC_{T} method (C_{T} values <30). Data are shown as means±S.D. based on three replicates.

TABLE 3
Primers used for real-time PCR

| Gene | Forward Primer | Reverse Primer |
|---|---|---|
| GAPDH | GAGTCAACGGATTTGGTCGT | GACAAGCTTCCCGTTCTCAG |
| K1 | CAGCATCATTGCTGAGGTCAAGG | CATGTCTGCCAGCAGTGATCTG |
| K3 | ACGTGACTACCAGGAGCTGATG | ATGCTGACAGCACTCGGACACT |
| K10 | CCTGCTTCAGATCGACAATGCC | ATCTCCAGGTCAGCCTTGGTCA |
| K12 | AGCAGAATCGGAAGGACGCTGA | ACCTCGCTCTTGCTGGACTGAA |
| PAX6 | TGTCCAACGGATGTGTGAGT | TTTCTCCAAGCAAAGATGGAC |

Lentiviral RNA

The PAX6 gene was targeted using lentiviral shRNAs that were cloned into a pLKO.1 plasmid between Age I and EcoR I. shRNA targeting sequences for gene-specific knockdowns were 5′-CGTCCATCTTTGCTTGGGAAA-3′ and 5′-AGTTTGAGAGAACCCATrATC-3′. In all gene knockdown experiments, we used a lentiviral pLKO.1-puro non-target shRNA control plasmid (Sigma, Inc.) encoding a shRNA that does not target any known genes from any species as a negative control. For preparation of lentiviral shRNA particles, replication-incompetent lentiviral particles were packaged in 293T cells by co-transfection of shRNA constructs with a packaging mixture (pCMV-dR8.2 and pCMV-VSVG at 9:1 ratio). Virus was harvested twice at 48 hrs and 72 hrs post-transfection. Cells were infected with the lentivirus for 16-20 hrs with fresh media containing individual virus and Polybrene at a final concentration of 8 μg/ml. The infected cells were further selected with 2 μg/ml puromycin for 48 hrs.

Immunofluorescence and Confocal Microscopy

Cells were fixed with 4% paraformaldehyde for 15 min at room temperature, permeabilized with phosphate buffered saline (PBS) containing 0.3% Triton X-100 for 10 min, and blocked in PBS containing 5% bovine serum albumin and 0.3% Triton X-100. The cells were incubated with primary antibodies for 18 hrs at 4° C., washed three times in PBS, and incubated with secondary antibody for 1 hr. Cell nuclei were counterstained with DAPI. Immunofluorescence of paraffin-embedded tissue sections was accomplished by standard de-paraffinization, followed by the same immunofluorescence protocol as described above.

The following antibodies were used: mouse anti-p63 monoclonal antibody, rabbit anti-K5 monoclonal antibody, and mouse anti-K10 monoclonal antibody (MA1-21871, RM2106S0, and MS611 P0, Thermo Fisher Scientific, Inc.); rabbit anti-PAX6 polyclonal antibody (PRB-278P, Covance, Inc.); mouse anti-K1 monoclonal antibody (sc-376224, Santa Cruz Biotechnology, Inc.); and mouse anti-K3/12 monoclonal antibody and rabbit anti-K12 monoclonal antibody (ab68260 and ab124975, Abcam, Inc.). Secondary antibodies, Alexa Fluor 488 or 568-conjugated anti-mouse or rabbit immunoglobulin G (IgG) (Invitrogen, Inc.), were used at a dilution of 1:500. Images were obtained using an Olympus FV1000 confocal microscope.

Microarray Data Analysis

Total RNA was isolated from LSCs and SESCs as in our previous study (15). Raw data were deposited in the Gene Expression Omnibus (GEO) database under accession number GSE32145. Microarray-based gene expression data produced in this study were normalized across samples and subjected to average linkage hierarchical clustering using the Cluster 3.0/Tree View software package (16). Selection of genes belonging to Wnt and Notch pathways was performed based on previous studies. Expression values were overlaid on a network generated using the GeneSet analysis tool for the Reactome network (17) using the Reactome Functional Interaction (FI) plugin for Cytoscape 3 (18).

Results

PAX6 and p63 Expression During Ocular Development in Mice

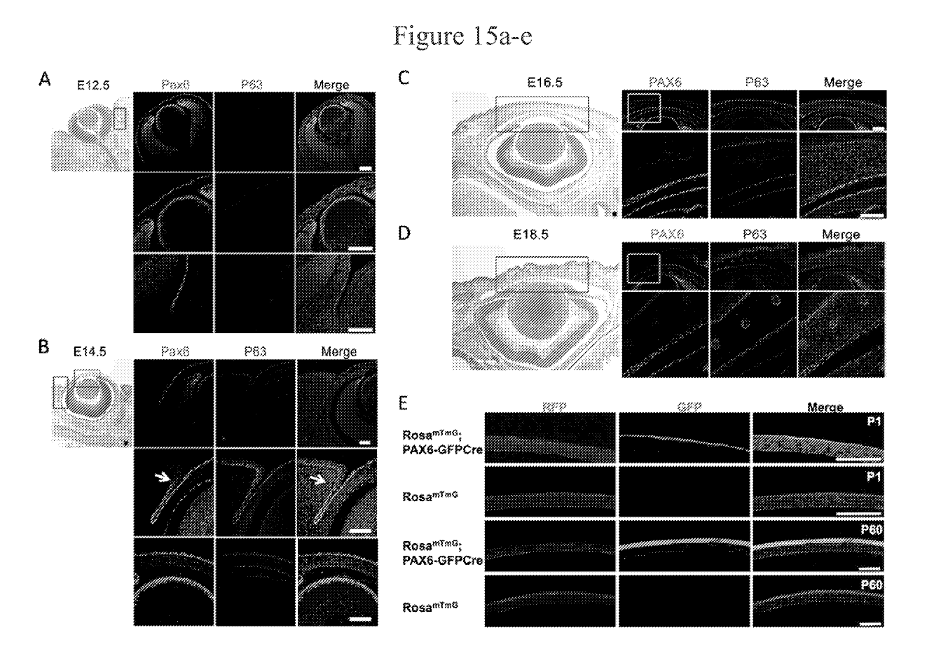

To elucidate the function of PAX6 and p63 during cornea development, we first studied their expression profiles from embryonic day (E) 12.5 to E18.5 in mouse embryos. At E12.5, strong PAX6 expression was detected in the eye field and particularly in the early corneal epithelium, whereas p63 expression was negative (FIG. 15A). In contrast, p63-positive cells appeared at the ocular surface and expanded to the limbus and cornea later, at E14.5, following the development and fusion of the eyelid (FIG. 15B). PAX6 expression was restricted to the eye field during ocular development (FIG. 15A-D). In addition, we performed lineage tracing experiments using a Pax6 promoter driving a GFP reporter. We observed intensive GFP expression in the corneal epithelium of ROSA^{mT/mG}; PAX6-GFPCre mice at P1 and P60 (FIG. 15E). These results suggest a central role of PAX6 in the limbal stem cells and the corneal epithelium, from early developmental stages to adulthood.

PAX6 and P63 Expression in Human Corneal and Skin Epithelium

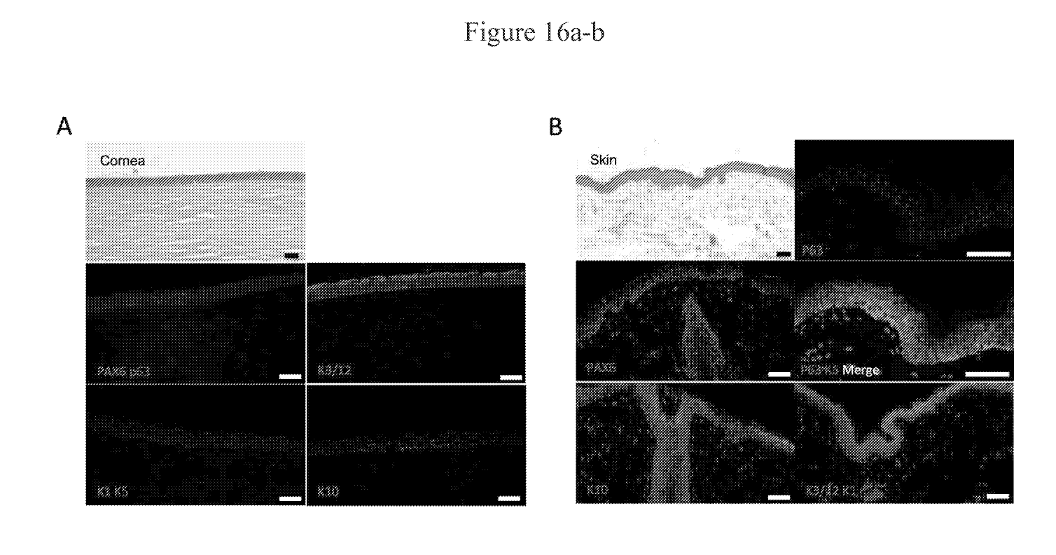

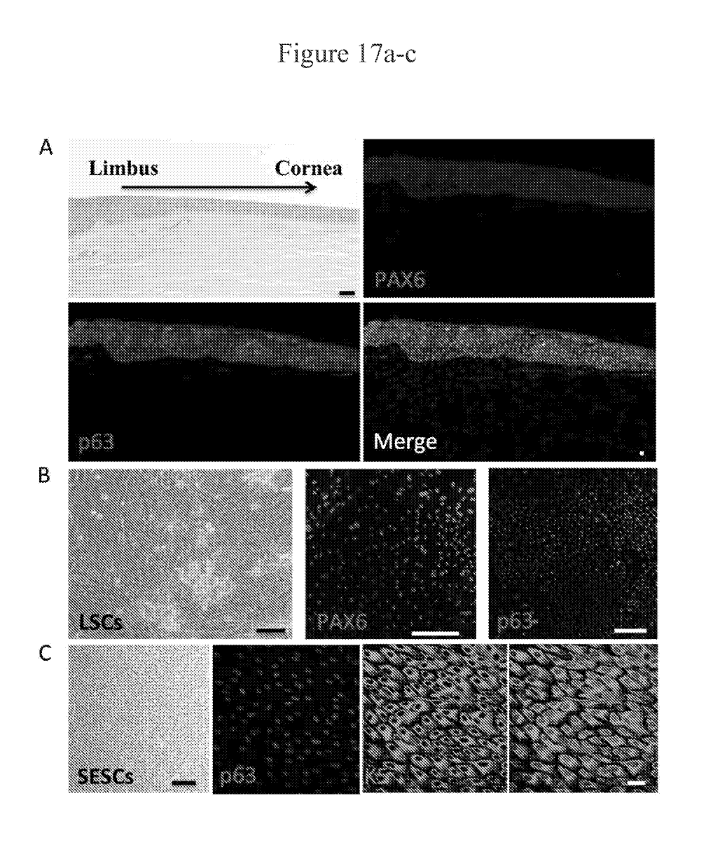

We observed that p63, a master regulator of squamous epithelial cell development, is mainly expressed in the basal layer of both the limbus and skin epidermis, suggesting the similarity of these two epithelial cell types. However, while PAX6 was highly expressed in the epithelial layers of the cornea, it was undetectable in the skin epidermis in adult humans (FIG. 16). For further characterization of skin epidermal epithelium and corneal epithelium, we performed immunostaining for tissue-specific keratins. LSCs migrate to the central cornea upon differentiation, with K3 and K12 as corneal specific markers (FIG. 16A), whereas skin epidermal-specific keratins, K1 and K10, are expressed in the suprabasal epidermal layers (FIG. 16B), and PAX6 and p63 co-localized in the corneal limbus (FIG. 17A). We further isolated and cultured LSCs and skin epidermal stem cells (SESCs) in vitro. LSCs could be expanded and identified by PAX6 and p63 expression (FIG. 17B), SESCs could be identified by p63 and K5 expression (FIG. 17C).

PAX6 is Essential in Corneal Cell Fate Determination

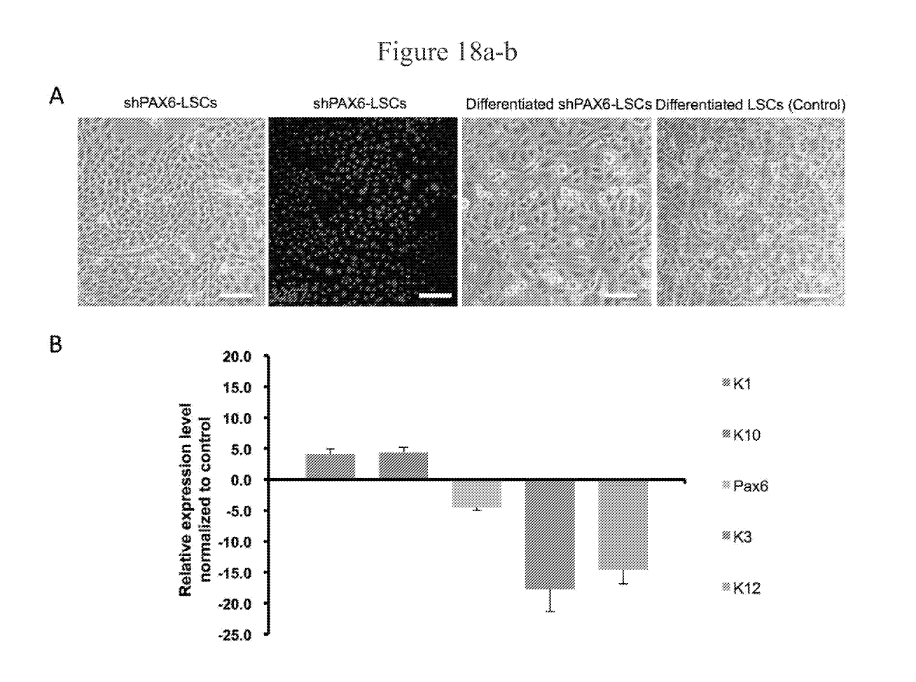

To investigate the role of PAX6 in determining corneal cell fate, we used a lentiviral-mediated PAX6 knockdown in human LSCs. We purified the PAX6 shRNA LSCs by puromycin selection. RNA was extracted from both stem cells and differentiated cells; the expression levels of related genes were compared by quantitative PCR. Two different shRNAs for PAX6 were used and showed similar results. Although 4.5-fold knockdown of PAX6 in LSCs did not produce a proliferation defect with active Ki67 expression (FIG. 18A), the corneal-specific markers K3 and K12 were significantly down-regulated upon differentiation by 17.7-fold and 14.5-fold (p<0.05), respectively, compared to controls. In contrast, skin-specific K1 and K10 expression was up-regulated by 4.1- and 4.4-fold (p<0.05), respectively (FIG. 18B). These results indicate that loss of PAX6 in LSCs leads to skin-like differentiation.

Loss of PAX6 in Human Congenital Limbal Dermoid Tissue

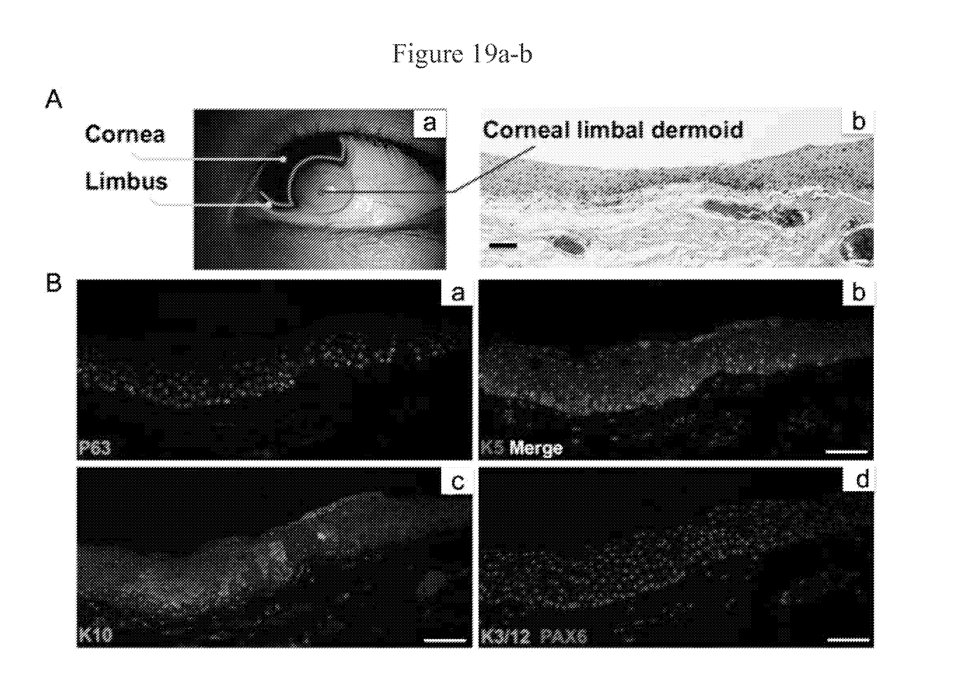

To determine the clinical relevance of PAX6 expression in LSCs and corneal epithelial cells (CECs), we studied human corneal limbal dermoids, which exhibited skin epidermis pathology with vascularization and disorganized cells in the stroma (FIG. 19A). We found that PAX6 expression was completely absent in an area of corneal dermoids (FIG. 19B), Moreover, we observed localized expression of p63 and K5 in the basal layer (FIG. 19B), and skin-specific keratins K1 and K10 in the suprabasal layer (FIG. 19B). These results suggest a conversion of cornea epithelial cells to skin-like epithelial cells in patient tissues during development and strongly support the essential role of PAX6 in corneal epithelial cell-fate determination.

Signaling Pathways in LSC Fate Determination

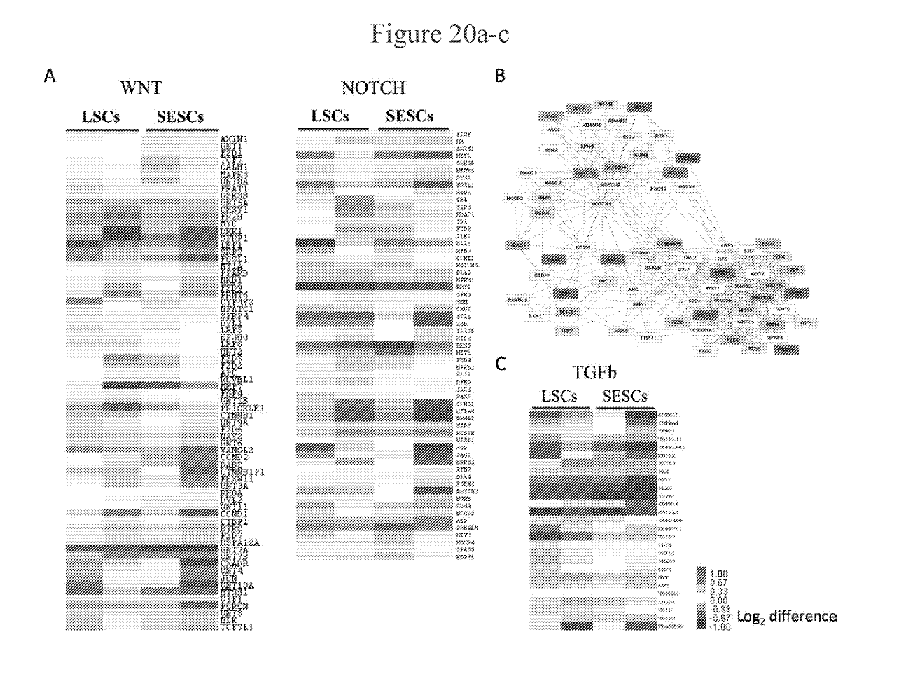

To further determine functional characteristics that control corneal cell-fate commitment, we sought to identify signaling pathways that might be differentially activated in LSCs and SESCs. We performed RNA expression analyses using microarrays on LSCs and SESCs, followed by gene ontology and pathway analyses using DAVID (19, 20). We identified subsets of genes that showed at least 2-fold expression differences between LSCs and SESCs, resulting in a total of 1185 genes. This analysis identified numerous GO terms and signaling pathways affecting many cellular and metabolic processes. In particular, however, Notch, Wnt and TGF-beta pathways emerged as important pathways from this analysis, consistent with their critical roles in the self-renewal and lineage commitment of stem cells from a variety of tissues (21-23), including the epithelium. Graphical representation of expression changes for distinct members of these pathways is provided in FIG. 20.

### DISCUSSION

A clear, transparent cornea is maintained by self-renewal of LSC's and their differentiation into CECs (24, 25). These two processes must be highly organized in order to maintain the integrity and homeostasis of the corneal epithelium. Pathological changes in LSCs can lead to a loss of transparency in the cornea and cause partial or complete blindness (2, 15). In this study, we found that PAX6 is essential for the maintenance of LSC characteristics and their further commitment to the CEC lineage. Of note, although p63 is well documented as a master gene of self-renewal and differentiation for common squamous epithelia such as that in cornea, epidermis and prostate (4-6), we observed that p63 was expressed after PAX6, which implicates PAX6 expression as a central event in corneal cell-fate control.

PAX6-deficient LSCs in culture exhibit a skin-like epithelium cell fate as indicated by a switch in keratin expression upon differentiation, specifically replacement of corneal-specific K3/K12 by skin-specific K1/K10. Furthermore, PAX6 is absent from corneal dermoid tissue, a congenital teratoma that switches cornea into the skin lineage. This is consistent with the recent finding of PAX6 down-regulation in abnormal epidermal differentiation, such as that seen in Stevens-Johnson syndrome, chemical burn, aniridia and recurrent pterygium (2).

Wnt and Notch signaling have been shown to be critical for the self-renewal and lineage commitment of epithelial cells in embryogenesis and stem cells from a variety of tissues (21-23). Both signaling pathways are complex, with different receptors, ligands, co-activators and inhibitory proteins. For example, Notch1 helps maintain corneal epithelial cell fate during repair in injured mice cornea (26). The influence of Wnt signaling on ocular surface development has also been extensively reported. Wnt4 is expressed in human fetal cornea and in adult basal LSCs (27). Dkk2, an antagonist of canonical Wnt signaling, is required for accurate development of the ocular surface epithelium in mice by regulation of Wnt/β-catenin activity (14). In addition, our previous work has suggested a central role of the Wnt7A-PAX6 axis in corneal epithelial cell fate determination, in which SESCs could be converted to LSC-like cells by overexpression of PAX6 (15). In the current study, we have provided further evidence for the importance of the Wnt and Notch signaling pathways by comparison of gene expression profiles between LSCs and SESCs. Further investigation to define the function of these genes in corneal epithelium specification may allow us to better understand and manipulate the corneal diseases.

Taken together, our data indicate a critical role of PAX6 in LSCs and corneal epithelial fate determination. Furthermore, we identified a key role for PAX6 in determination of the corneal epithelial phenotype. Understanding how PAX6 controls corneal cell fate will provide important insight into corneal homeostasis and disease and aid in developing new therapeutic strategies for the treatment of common corneal diseases.

### REFERENCES

1. Masse, K., Bhamra, S., Eason, R., Dale, N., and Jones, E. A. (2007). Purine-mediated signalling triggers eye development. Nature 449, 1058-1062.
2. Li, W., Chen, Y. T., Hayashida, Y., Blanco, G., Kheirkah, A., He, H., Chen, S. Y., Liu, C. Y., and Tseng. S. C. (2008). Down-regulation of Pax6 is associated with abnormal differentiation of corneal epithelial cells in severe ocular surface diseases. J. Pathol. 214, 114-122.
3. Zhao, B., Allinson, S. L., Ma, A., Bentley, A. J., Martin, F. L., and Fullwood, N. J. (2008). Targeted cornea limbal stem/progenitor cell transfection in an organ culture model. Invest. Ophthalmol. Vis. Sci. 49, 3395-3401.
4. Koster, M. I., Kim, S., Mills, A. A., DeMayo, F. J., and Roop, D. R. (2004). p63 is the molecular switch for initiation of an epithelial stratification program. Genes Dev. 18, 126-131.
5. Pellegrini, G., Dellambra, E., Golisano, O., Martinelli, E., Fantozzi, I., Bondanza, S., Ponzin, D., McKeon, F., and De Luca. M. (2001). p63 identifies keratinocyte stem cells. Proc. Natl. Acad. Sci. U.S.A. 98, 3156-3161.
6. Mills, A. A., Zheng, B., Wang, X. J., Vogel, H., Roop. D. R., and Bradley. A. (1999). p63 is a p53 homologue required for limb and epidermal morphogenesis. Nature 398, 708-713.
7. Yang, A., Schweitzer, R., Sun, D., Kaghad, M., Walker, N., Bronson, R. T., Tabin, C., Sharpe, A., Caput, D., Crum, C., and McKeon, F. (1999). p63 is essential for regenerative proliferation in limb, craniofacial and epithelial development. Nature 398, 714-718.
8. Arwert, E. N., Hoste, E., and Watt, F. M. (2012). Epithelial stem cells, wound healing and cancer. Nat. Rev. Cancer 12, 170-180.
9. Blanpain, C., and Fuchs, E. (2009). Epidermal homeostasis: a balancing act of stem cells in the skin. Nat. Rev. Mol. Cell Biol. 10, 207-217.
10. Kopan, R., and Fuchs, E. (1989). A new look into an old problem: keratins as tools to investigate determination, morphogenesis, and differentiation in skin. Genes Dev. 3, 1-15.
11. Schlotzer-Schrehardt, U., and Kruse, F. E. (2005). Identification and characterization of limbal stem cells. Exp. Eye Res. 81, 247-264.
12. Eichner, R., Bonitz, P., and Sun, T. T. (1984). Classification of epidermal keratins according to their immunoreactivity, isoelectric point, and mode of expression. J. Cell Biol. 98, 1388-1396.
13. Ramaesh, T., Collinson. J. M., Ramaesh, K., Kaufman. M. H., West, J. D., and Dhillon, B. (2003). Corneal abnormalities in Pax6⁺ small eye mice mimic human aniridia-related keratopathy. Invest. Ophthalmol. Vis. Sci. 44, 1871-1878.
14. Mukhopadhyay, M., Gorivodsky, M., Shtrom, S., Grinberg, A., Niehrs, C., Morasso, M. I., and Westphal, H. (2006). Dkk2 plays an essential role in the corneal fate of the ocular surface epithelium. Development 133, 2149-2154.
15. Ouyang, H., Xue. Y., Lin, Y., Zhang, X., Xi, L., Patel, S., Cai, H., Luo, J., Zhang. M., Zhang, M., Yang, Y., Li, G., Li, H., Jiang, W., Yeh, E., Lin, J., Pei, M., Zhu, J., Cao, G., Zhang, L., Yu, B., Chen, S., Fu, X. D., Liu, Y., and Zhang, K. (2014). WNT7A and PAX6 define corneal epithelium homeostasis and pathogenesis. Nature 511, 358-361.
16. de Hoon, M. J., Imoto, S., Nolan, J., and Miyano, S. (2004). Open source clustering software. Bioinformatics 20, 1453-1454.
17. Wu, G., Feng, X., and Stein, L. (2010). A human functional protein interaction network and its application to cancer data analysis. Genome Biol. 11, R53.
18. Shannon, P., Markiel, A., Ozier, O., Baliga, N. S., Wang, J. T., Ramage, D., Amin, N., Schwikowski, B., and Ideker, T. (2003). Cytoscape: a software environment for integrated models of biomolecular interaction networks. Genome Res. 13, 2498-2504.
19. Huang da, W., Sherman, B. T., and Lempicki, R. A. (2009). Systematic and integrative analysis of large gene lists using DAVID bioinformatics resources. Nat. Protoc. 4, 44-57.
20. Huang da, W., Sherman, B. T., and Lempicki, R. A. (2009). Bioinformatics enrichment tools: paths toward the comprehensive functional analysis of large gene lists. Nucleic Acids Res. 37, 1-13.
21. Lien, W. H., and Fuchs, E. (2014). Wnt some lose some: transcriptional governance of stem cells by Wnt/β-catenin signaling. Genes Dev. 28, 1517-1532.
22. Collu, G. M., Hidalgo-Sastre, A., and Brennan, K. (2014). Wnt-Notch signalling crosstalk in development and disease. Cell. Mol. Life Sci. 71, 3553-3567.
23. Kopan, R., and Ilagan, M. X. (2009). The canonical Notch signaling pathway: unfolding the activation mechanism. Cell 137, 216-233.
24. Cotsarelis, G., Cheng, S. Z., Dong, G., Sun, T. T., and Lavker, R. M. (1989). Existence of slow-cycling limbal epithelial basal cells that can be preferentially stimulated to proliferate: implications on epithelial stem cells. Cell 57, 201-209.
25. Davanger, M., and Evensen, A. (1971). Role of the pericorneal papillary structure in renewal of corneal epithelium. Nature 229, 560-561.
26. Figueira, E. C., Di Girolamo, N., Coroneo, M. T., and Wakefield, D. (2007). The phenotype of limbal epithelial stem cells. Invest. Ophthalmol. Vis. Sci. 48, 144-156.
27. Vauclair, S., Majo, F., Durham, A. D., Ghyselinck, N. B., Barrandon, Y., and Radtke, F. (2007). Corneal epithelial cell fate is maintained during repair by Notch1 signaling via the regulation of vitamin A metabolism. Dev. Cell 13, 242-253.
28. Muzumdar, M. D., Tasic, B. Miyamichi, K., Li, L., and Luo, L. (2007) A global double-fluorescent Cre reporter mouse. Genesis 45, 593-605.
29. Rowan, S., Conley, K. W., Le, T. T., Donner, A. L., Maas, R. L., and Brown, N. L. (2008). Notch signaling regulates growth and differentiation in the mammalian lens. Dev. Biol. 321, 111-122.

### Example 3

A Method for Preparing Donor Corneal Repair Material

1. Materials

Limbal stem cell culture medium or limbal stem cell maintenance medium: DMEM/nutrient mixture F-12 (volume:volume of DMEM:F-12 at 3:1 ratio) basic medium, supplemented with the following: 10% fetal bovine serum, 0.4 μg/ml hydrocortisone, 10⁻¹⁰ M cholera toxin, 5 g/ml transferrin, 2×10⁻⁹ M 3,3′,5-triiodo-L-thyronine, 5 μg/ml insulin, 10 ng/ml epidermal growth factor (EGF), 100 U/ml penicillin and 100 tμg/ml streptomycin. After cell passage 4, a ROCK inhibitor is added to the limbal stem cell culture medium to maintain LSCs in a proliferative state, such as by the addition of 1 μM Y-27632. The above components, DMEM, F12 medium and fetal bovine serum are purchased from GIBCO® (Life Technologies), the remaining components are purchased from Sigma, USA.

Limbal stem cell differentiation medium: CnT-30 or equivalent (CelInTec Advanced Cell Systems AG, Bern, Switzerland). Other limbal stem cell differentiation media which could be used in place of CnT-30 are CnT-02, CnT-02-3DP5, RegES (Regea 06/015, Regea 07/046, and Regea 08/013; Rajala et al., 2010, PLOS One 5(4):e10246).

Eyeballs: from the donors.

2. Methods

Limbal stem cell isolation and expansion: Specimens of the limbus were isolated from eyeballs, washed with 100 IU/ml penicillin and 100 μg/ml streptomycin in PBS, chopped to small pieces (around 2×2 mm² tissue sizes), and digested with 0.2% collagenase IV at 37° C. for one hour. Then, single cells are obtained by further treatment with 0.25% trypsin and 1 mM ethylenediaminetetraacetic acid (EDTA) at 37° C. for 15 minutes. Primary cells were seeded on plastic dishes coated with 2% growth factor reduced (GFR) Matrigel® matrix (BD Biosciences catalog no. 354230). Cells are cultured in limbal stem cell culture medium with no feeder cells (feeder-free) and passaged at 70-90% confluence to 15-20%/confluence after passage.

Corneal repair material for transplantation: A suspension of passage 3 (P3) generation of cultured limbal stem cells (also called human corneal stem cells) at 2×10⁴ cells is mixed with Matrigel® in a final volume of 50 μl to form a three dimensional cell culture, and the Matrigel®-embedded corneal stem cells are cultured in differentiation-inducing medium CnT-30 (CelInTec Advanced Cell Systems AG, Bern, Switzerland) for 14 days to differentiate to corneal epithelial cells as donor cells for transplantation.

### Example 4

A method for preparing donor corneal repair material

1. Materials

Same as in Example 3.

2. Methods

Limbal stem cell isolation and expansion: Specimens of the limbus were isolated from eyeballs, washed with 100 IU/ml of penicillin and 100 μg/ml streptomycin in PBS, chopped to small pieces (around 2×2 mm² tissue sizes), and digested with 0.2% collagenase IV at 37° C. for two hours. Then, single cells are obtained by further treatment with 0.25% trypsin and 1 mM EDTA at 37° C. for 15 minutes. Primary cells were seeded on plastic dishes coated with 2% growth factor reduced Matrigel® matrix (BD Biosciences catalog no. 354230). Cells are cultured in limbal stem cell culture medium with no feeder cells (feeder-free) and passaged at 70-90% confluence to 15-20% confluence after passage.

Preparation of cell source for transplantation: A suspension of passage 3 (P3) generation cultured human LSCs at 4×10⁴ cells is mixed with Matrigel® in a final volume of 50 μl, and the embedded LSCs are cultured in corneal epithelial differentiation-inducing medium CnT-30 (CelInTec Advanced Cell Systems AG, Bern, Switzerland) for 3 days to obtain transplantation donor cells.

### Example 5

A method for preparing donor corneal repair material

1. Materials

Same as in Example 3.

2. Methods

Limbal stem cell isolation and expansion: Limbal tissue is dissected from eyeball and washed with 100 IU/ml of penicillin and 100 μg/ml streptomycin in PBS. Limbal tissue is cut into 2 mm×2 mm pieces and incubated and digested with 0.2% collagenase IV at 37° C. for 2.5 hours, then treated with 0.25% trypsin and 1 mM EDTA at 37° C. for 20 minutes to obtain a single cell suspension. These digested primary cells are then seeded in a Matrigel®-coated dish (growth factor reduced Matrigel®; BD Biosciences catalog no. 354230). Cells are cultured in limbal stem cell culture medium without feeder cells (feeder-free) to expand LSCs, passaging at 70-90% confluence to 15-20% confluence after passage.

Preparation of cell source for transplantation: A suspension of third passage cultured human corneal stem cells at 1×10⁴ is mixed with collagen in CnT-30 medium (CelInTec Advanced Cell Systems AG, Bern, Switzerland) at a final volume of 50 μl, and the collagen-embedded corneal stem cells are incubated in differentiation-inducing medium CnT-30 (CelInTec Advanced Cell Systems AG, Bern, Switzerland) for 18 days to obtain donor corneal repair material.

### Example 6

A method for preparing donor corneal repair material

1. Materials

Same as in Example 3.

2. Methods

Limbal stem cell isolation and expansion: Specimens of the limbus were isolated from eyeballs, washed with 100 IU/ml of penicillin and 100 μg/ml streptomycin in PBS, chopped into small pieces (around 2 mm×2 mm) of tissues, and digested with 0.2% collagenase IV 37° C. for 3.5 hours. Then, the cells and cell masses are further treated with 0.25% trypsin and 1 mM EDTA at 37° C. for 10 minutes to obtain a single cell suspension. The single primary cells are then cultured in dishes coated with 2% growth factor reduced Matrigel® (BD Biosciences catalog no. 354230). Cells are cultured in limbal stem cell culture medium without feeder-cells (Feeder-free) to expand LSCs, passaging at 70-90% confluence to 15-20% confluence after passage. Typically, ROCK inhibitor, Y-27632 is added to LSC culture medium after P4 to maintain LSC proliferation.

Preparation of cell source for transplantation: A suspension of third passage cultured human corneal stem cell at 3×10⁴ is mixed with amniotic membrane in CnT-30 medium (CelInTec Advanced Cell Systems AG, Bern, Switzerland) at a final volume of 50 μl. The amniotic membrane-treated human corneal stem cell mixture is placed in culture in differentiation-inducing medium CnT-30 medium for 14 days to obtain donor corneal repair material.

Although third passage (P3) LSCs are used in Examples 3-6, other LSC passages may also be used. For LSCs obtained after passage 4, these LSCs would have been maintained in LSC culture medium or maintenance medium supplemented with a ROCK inhibitor, such as Y-27632 used at a concentration of 1 μM Y-27632.

3-D Differentiation of Cultured LSCs.

Plastic plates are treated by coating with 2% growth factor reduced Matrigel® (354230, BD Biosciences, Inc.) for 30 min at 37° C., before seeding LSC cells. LSC culture medium or maintenance medium is: DMEM/F12 and DMEM (1:1) with 1/100 Pen-Strep, 10% Fetal bovine serum, 10 ng/ml EGF, 5 μg/ml insulin, 0.4 μg/ml hydrocortisone, 10⁻¹⁰ M cholera toxin and 2×10⁻⁹ M 3,3′,5-triiodo-L-thyronine to which is added 1 μM Y-27632 only when the LSC cells are growing slowly. Cells are passaged at 70-90% confluence, and for continuous culture in growth factor reduced Matrigel®-coated dishes, cell confluence immediately after passage is about 15-20% confluence. To differentiate LSC's in vitro, LSC's are dissociated to obtain a single cell suspension and the individual LSCs are embedded in Matrigel® at 2×10⁴ cells/50 μl gel. 3-D structures were formed after 14-18 day culture in a differentiation medium CnT-30 (CelInTec Advanced Cell Systems AG, Bern, Switzerland) or an equivalent differentiation medium which supports differentiation of LSCs to CECs.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

1. An isolated limbal stem or progenitor cell (LSC) population or LSC-like population comprising a chemically synthesized, recombinant or isolated nucleic acid encoding PAX6 integrated into a chromosome, or as an extrachromosomal genetic material, wherein the isolated LSC population is substantially free of non-LSC cells or wherein the LSC-like population is substantially free of non-LSC-like cells, or wherein the isolated LSC or LSC-like population is substantially free of non-LSC and non-LSC-like cells.

2. The isolated LSC population or LSC-like population of claim 1, wherein the chemically synthesized, recombinant or isolated nucleic acid can express PAX6 or a fragment thereof, wherein PAX6 or a fragment thereof can maintain LSC or LSC-like state or can direct a stem cell or progenitor cell to a LSC or LSC-like state, and wherein LSC or LSC-like state restricts a cell population to a differentiation pathway resulting in corneal epithelial cells (CECs).

3. The isolated LSC population or LSC-like population of claim 2, wherein the chemically synthesized, recombinant or isolated nucleic acid expresses PAX6 or a fragment thereof, wherein PAX6 or a fragment thereof maintains LSC or LSC-like state or directs a stem cell or progenitor cell to a LSC or LSC-like state, and wherein LSC or LSC-like state restricts a cell population to a differentiation pathway resulting in corneal epithelial cells.

4. An isolated skin epithelial stem cell (SESC) population or SESC-like population comprising a chemically synthesized, recombinant or isolated nucleic acid encoding PAX6 integrated into a chromosome, or alternatively, not integrated remaining as an extrachromosomal genetic material, wherein the isolated SESC population is substantially free of non-SESC cells, or wherein the SESC-like population is substantially free of non-SESC-like cells, or wherein the isolated SESC or SESC-like population is substantially free of non-SESC and non-SESC-like cells, or wherein the isolated SESC or SESC-like population is substantially free of non-SESC, non-SESC-like, non-LSC and non-LSC-like cells.

5. The isolated SESC or SESC-like population of claim 4, wherein the chemically synthesized, recombinant or isolated nucleic acid can express PAX6 or a fragment thereof, wherein PAX6 or a fragment thereof can maintain LSC or LSC-like state or can direct a stem cell or progenitor cell to a LSC or LSC-like state, and wherein LSC or LSC-like state restricts a cell population to a differentiation pathway resulting in corneal epithelial cells.

6. The isolated SESC or SESC-like population of claim 5, wherein the chemically synthesized, recombinant or isolated nucleic acid expresses PAX6 or a fragment thereof, wherein PAX6 or a fragment thereof directs SESC or SESC-like cell to a LSC or LSC-like state, wherein LSC or LSC-like state restricts a cell population to a differentiation pathway resulting in corneal epithelial cells.

7. A pharmaceutical composition comprising the LSC population or LSC-like population of claim 1 and a suitable carrier.

8. A pharmaceutical composition comprising the SESC population or SESC-like population of claim 4 and a suitable carrier.

9-12. (canceled)

13. The LSC population or LSC-like population of claim 1, that differentiates into a corneal epithelial cell population.

14-17. (canceled)

18. The SESC population or SESC-like population of claim 4, which differentiates into corneal epithelial cells.

19. (canceled)

20. The LSC population or LSC-like population of claim 1, wherein 90-95% of the LSC population or LSC-like population expresses p63, PAX6, K19 and Ki67.

21. The LSC population or LSC-like population of claim 1, wherein less than 5% of the LSC population expresses K5 and K14.

22. The LSC population of claim 1, wherein greater than 95% of the LSC population expresses WNT7A and FZD5.

23. The LSC-like population of claim 1, wherein less than 5% of the LSC-like population expresses WNT7A.

24. The SESC population or SESC-like population of claim 4, wherein 90-95% of the cell population expresses p63, K5 and Ki67 while remaining in a SESC or SESC-like cell fate.

25. The SESC population or SESC-like population of claim 4, wherein K3 or K12 expression is not detected in cells remaining in a SESC or SESC-like cell fate.

26. The SESC population of claim 4, wherein WNT7A is expressed in cells remaining in a SESC or SESC-like cell fate at about 4-5 fold lower level than the level in LSC cells.

27. The SESC population or SESC-like population of claim 4, wherein PAX6 is not expressed or expressed in cells remaining in a SESC or SESC-like cell fate at a level less than about one eighth of the level in LSC cells.

28. The SESC-like population of claim 4, wherein WNT7A is expressed in more than 70% of cells remaining in a SESC-like fate.

29. (canceled)

30. The LSC population or LSC-like population of claim 2, wherein the corneal epithelial cells express PAX6 and corneal epithelial markers, K3 and K12.

31-42. (canceled)

43. Tissue comprised of the cells of claim 1 or 4.

44. (canceled)

45. A method of regenerating or repairing tissue in a subject comprising introducing the cell of claim 1 or 4 into or onto a subject in a sufficient amount to regenerate or repair tissue.

46. The method of claim 45, wherein the tissue regenerated or repaired comprises tissues of corneal epithelial cell lineage comprising limbal stem or progenitor cell (LSC) and corneal epithelial cell.

47. A method for obtaining limbal stem cell or progenitor (LSC)-like cells from skin epithelial stem cells (SESCs) of a subject, the method comprising introduction of a PAX6 gene or up-regulating PAX6 gene expression in SESCs, in order to increase PAX6 protein in SESCs to a sufficient level so as to convert SESCs to LSC-like cells, thereby obtaining LSC-like cells from SESCs of a subject.

48. The method of claim 47, wherein introduction of a PAX6 gene or up-regulating PAX6 gene expression in SESCs for obtaining limbal stem or progenitor cell (LSC)-like cells from skin epithelial stem cells (SESCs) of a subject comprises:
(a) obtaining SESCs from the subject;
(b) culturing the SESCs in a feeder-free cell culture in vitro or ex vivo;
(c) introducing at least one PAX6 gene or up-regulating PAX6 gene expression in the SESCS so as to increase PAX6 protein in SESCs to a sufficient level so as to convert SESCs to limbal stem cell or progenitor (LSC)-like cells, thereby obtaining mammalian limbal stem cell or progenitor (LSC)-like cells from skin epithelial stem cells (SESCs) from a subject.

49. The method of claim 47, wherein the method is an in vitro method, ex vivo method or in situ or directly applied on a subject.

50. The method of claim 47, wherein the subject is treated with an agent that introduces a nucleic acid encoding PAX6 protein, up-regulates PAX6 gene expression, or increases PAX6 activity.

51. The method of claim 47, wherein the agent comprises a gene therapy vector, viral particle, lentivirus, adenovirus, adeno-associated virus, recombinant nucleic acid, recombinant protein, PAX6 protein, small molecule regulator of PAX6 expression, inhibitor of a negative regulator of PAX6 expression, a small molecule inhibitor of a negative regulator of PAX activity, a small molecule enhancer of PAX6 activity, or a combination thereof.

52. The method of claim 47, wherein the PAX6 gene is selected from a set of PAX6a gene, PAX6b gene, engineered PAX6a gene, engineered PAXb gene, any member of the PAX6 gene family, nucleic acid encoding all or part of PAX6a protein, nucleic acid encoding all or part of PAX6b protein, and any nucleic acid encoding a protein with PAX6 or PAX6-like activity.

53. (canceled)

54. The method of claim 52, wherein a protein with PAX6 or PAX6-like activity is any protein which can cause increased expression of endogenous K19, wherein the K19 upregulated SECS may differentiate to corneal epithelial cell (CEC) or CEC-like cells with increased expression of K3 and K12 genes and decreased expression of K1 and K10 genes.

55. A method for obtaining corneal epithelial cell (CEC)-like cells from LSC-like cells of claim 48, further comprising differentiating cells of (c) in a feeder-free LSC differentiation medium so as to convert LSC-like cells to CEC-like cells.

56. The method of claim 55, wherein the feeder-free LSC differentiation medium is chemically defined, xeno-free or free of components other than components derived from the same species as the culture cells, serum-free, and/or devoid of any animal or human product.

57-69. (canceled)

70. A method for obtaining and expanding in vitro mammalian limbal stem or progenitor cells (LSCs) from a subject in a feeder-free LSC culture medium, wherein the method comprises:
(a) obtaining a sample of tissue from the limbus region of an eye from the subject;
(b) dissociating the tissue so as to obtain single cells; and
(c) culturing single cells of (b) in a feeder-free cell culture medium so as to permit LSCs to proliferate, wherein the proliferated LSCs have a potential to differentiate into corneal epithelial cells (CECs), thereby obtaining and expanding in vitro mammalian limbal stem cells from a subject.

71. The method of claim 70, wherein in (c), single cells are cultured on a matrix or an extracellular matrix selected from the group consisting of Matrigel® or its equivalent, growth factor reduced Matrigel® or its equivalent, collagen, collagen IV, collagen IV sheet, mammalian amniotic membrane, human amniotic membrane, fibrinogen, thrombin, perlecan, laminin, fibronectin, recombinant fibronectin, proteoglycan, procollagens, hyaluronic acid, entactin, heparan sulfate, tenascin, poly-L-lysine, gelatin, poly-L-ornithine, extracellular matrix proteins, thrombin sheet, fibrinogen and thrombin sheet, and any combination thereof.

72. (canceled)

73. The method of claim 70, further comprising step (d), wherein the proliferated LSCs or LSC-like cells are passaged at 70-90% confluence before passage and 15-20% confluence after passage.

74-77. (canceled)

78. The method of claim 70, wherein the limbus region comprises corneal limbus of an eye, margin between cornea and conjunctiva, border of cornea and sclera, corneoscleral limbus, a region comprising interpalisade rete ridge, or a region comprising Palisades of Vogt.

79. The method of claim 70, wherein dissociating the tissue in (b) comprises treating with a dissociation agent or agents wherein the dissociation agent or agents is an equipment or tool to mechanically dissociate tissue to smaller masses and single cells, enzyme, protease, a chemical, a metal chelator, laser or combination thereof.

80-81. (canceled)

82. The method of claim 70, wherein the feeder-free cell culture medium comprises a minimum essential medium, a growth factor, a hormone, and a soluble factor.

83. The method of claim 82, wherein the feeder-free cell culture medium further comprises serum, preferably serum from a species from which the LSCs are being obtained and expanded or a serum substitute.

84. The method of claim 82, wherein the feeder-free cell culture medium further comprises Leukemia Inhibitory Factor (LIF) and/or a rho-associated protein kinase (ROCK) inhibitor selected from the group consisting of (R)-(+)-trans-4-(1-aminoethyl)-N-(4-Pyridyl)cyclohexanecarboxamide dihydrochloride monohydrate (Y-27632), 5-(1,4-diazepan-1-ylsulfonyl) isoquinoline (fasudil or HA 1077), H-1152, H-1152P, (S)-(+)-2-Methyl-1-[(4-methyl-5-isoguinolinyl)sulfonyl]homopiperazine dihydrochloride, Dimethylfasudil (diMF; H-1152P), N-(4-Pyridyl)-N′-(2,4,6-trichlorophenyl)urea, Y-39983, Wf-536, SNJ-1656, and (S)-(+)-2-methyl-1-[(4-methyl-5-isoguinolinyl)sulfonyl]-hexahydro-1H-1,4-diazepine dihydrochloride (H-1152), imidazole-containing benzodiazepines, imidazopyridine derivative, compound comprising an indazole core, a 2-aminopyridine/pyrimidine core, a 9-deazaguanine derivative, benzamide, or aminofurazan, and derivative and analog thereof, and a combination thereof.

85. (canceled)

86. The method of claim 84, wherein the LIF and/or ROCK inhibitor is added to the feeder-free cell culture medium after passage four (4) of LSCs, following isolation of LSCs from the subject.

87-88. (canceled)

89. The method of claim 70, wherein the feeder-free cell culture medium comprises DMEM/F12 medium, DMEM, penicillin-streptomycin, serum, EGF, insulin, hydrocortisone, cholera toxin, 3,3′,5-triiodo-L-thyronine, or combination thereof, wherein serum may be fetal bovine serum but preferably serum from same species as LSC being cultured or a serum substitute.

90-94. (canceled)

95. The method of claim 70, wherein LSCs express a set of markers comprising WNT7A, FZD5, PAX6, p63, keratin 5 (K5), keratin 14 (K14), keratin 19 (K19) and Ki67.

96. The method of claim 70, wherein 90-95% of the LSCs express p63, PAX6, K19 and Ki67.

97. The method of claim 70, wherein less than 5% of the LSCs express K5 and K14.

98. The method of claim 70, wherein greater than 95% of the LSCs express WNT7A and FZD5.

99-116. (canceled)

117. A method for treating a subject with a disease associated with malfunctioning limbal stem cells or corneal epithelial cells, wherein the method comprises:
transplanting LSCs, or LSC-like cells of claim 1 to an affected eye of a subject, wherein the transplanted cells populate cornea or limbus of the affected eye of the subject and restore normal cornea clarity and transparency,
thereby treating the subject with the disease associated with malfunctioning limbal stem or progenitor cells or corneal epithelial cells.

118. A method for treating a subject with a disease associated with malfunctioning limbal stem cells or corneal epithelial cells, wherein the method comprises:
transplanting LSCs, LSC-like, CECs or CEC-like cells produced by the method of claim 48 or 55 an affected eye of a subject, wherein the transplanted cells populate cornea or limbus of the affected eye of the subject and restore normal cornea clarity and transparency,
thereby treating the subject with the disease associated with malfunctioning limbal stem or progenitor cells or corneal epithelial cells.

119. The method of claim 117, wherein the disease or condition is a deficiency of limbal stem or progenitor cells, a deficiency of corneal epithelial cells, damage to corneal limbus, damage to cornea of an eye, damage to limbal stem cells, damage to corneal epithelial cells, congenital defect affecting corneal development or function, acquired defect affecting corneal development or function, congenital defect affecting cell fate determination switching corneal to skin lineage, acquired defect affecting cell fate determination switching corneal to skin lineage, abnormal epidermal differentiation, Stevens-Johnson syndrome, aniridia, recurrent pterygium, corneal disease, corneal epithelium squamous metaplasia, inflammatory keratopathy, trauma, chemical burns, alkaline burn, partial blindness, or complete blindness.

120-128. (canceled)

129. A method to determine if a patient with ocular metaplasia may benefit from treatment with PAX6 gene or gene product, wherein the method comprises assessing gene expression or protein level of WNT7A, PAX6, K3 and/or K12 in area of metaplasia, absence of WNT7A, PAX6, K3 and/or K12 in area of metaplasia indicates the patient with ocular metaplasia may benefit from treatment with PAX6 gene or gene product or cells of claim 1 or 4.

130-150. (canceled)

151. A kit comprising LSCs, LSC-like, CECs or CEC-like cells produced by the method of claim 48 or 55, or combination of said cells thereof, culture media and supplements, a packing material and an instruction for use.

152. The kit of claim 151 further comprising a pharmaceutically acceptable carrier.

153. The kit of claim 152, wherein the pharmaceutically acceptable carrier is a contact lens or its equivalent used to support cell attachment or growth in a curvature as curvature of a human or animal eye.

154. The kit of claim 153 further comprising human amniotic membrane or animal amniotic membrane.

155. A method for assessing risk of developing an eye disease affecting cornea or cornea function in a subject, wherein said method comprises assessing activity of WNTZ7A, FZD5 and PAX6 or combination thereof, in LSCs or corneal epithelial cells, wherein lower activity or no activity of WNTZ7A, FZD5 and PAX6 or combination thereof indicates a higher risk of developing an eye disease affecting the cornea or cornea function in a subject, thereby assessing the risk of developing an eye disease affecting cornea or cornea function in a subject.

156-186. (canceled)

187. A method for obtaining and expanding skin epithelial stem cell (SESC) in vitro from a subject in a feeder-free cell culture medium, wherein the method comprises:
(a) obtaining a sample of tissue from the interfollicular epidermis or any SESC stem cell niche harboring SESCs in the subject;
(b) dissociating the tissue so as to obtain single cells; and
(c) culturing single cells of (b) in a feeder-free cell culture medium so as to permit SESCs to proliferate, wherein the proliferated SESCs have a potential to differentiate into skin epidermal cells, thereby obtaining and expanding in vitro skin epithelial stem cells in vitro from a subject.

188. (canceled)

189. The method of claim 187, further comprising differentiating culturing isolated SESC or SESC-like cells in CnT-02 or equivalent medium which support differentiation of SESC or SESC-like cells to skin epidermal cells or skin epidermal-like cells, thereby obtaining skin epidermal cells or skin epidermal-like cells from SESC or SESC-like cells in vitro.

190-196. (canceled)


SEQUENCE LISTING (64 sequences)

SEQ ID NO: 1 - DNA, 22 bp, Artificial Sequence
  CASZ1 Forward Primer
gttctacggacagaagaccacg

SEQ ID NO: 2 - DNA, 22 bp, Artificial Sequence
  CASZ1 Reverse Primer
tcttgaagccgtccttggcgta

SEQ ID NO: 3 - DNA, 20 bp, Artificial Sequence
  FGFR3 Forward Primer
agtggagcctggtcatggaa

SEQ ID NO: 4 - DNA, 22 bp, Artificial Sequence
  FGFR3 Reverse Primer
ggatgctgccaaacttgttctc

SEQ ID NO: 5 - DNA, 22 bp, Artificial Sequence
  FZD5 Forward Primer
tggaacgcttccgctatcctga

SEQ ID NO: 6 - DNA, 22 bp, Artificial Sequence
  FZD5 Reverse Primer
ggtctcgtagtggatgtggttg

SEQ ID NO: 7 - DNA, 20 bp, Artificial Sequence
  GAPDH Forward Primer
gagtcaacggatttggtcgt

SEQ ID NO: 8 - DNA, 20 bp, Artificial Sequence
  GAPDH Reverse Primer
gacaagcttcccgttctcag

SEQ ID NO: 9 - DNA, 22 bp, Artificial Sequence
  ID2 Forward Primer
ttgtcagcctgcatcaccagag

SEQ ID NO: 10 - DNA, 22 bp, Artificial Sequence
  ID2 Reverse Primer
agccacacagtgctttgctgtc

SEQ ID NO: 11 - DNA, 23 bp, Artificial Sequence
  K1 Forward Primer
cagcatcattgctgaggtcaagg

SEQ ID NO: 12 - DNA, 22 bp, Artificial Sequence
  K1 Reverse Primer
catgtctgccagcagtgatctg

SEQ ID NO: 13 - DNA, 22 bp, Artificial Sequence
  K3 Forward Primer
acgtgactaccaggagctgatg

SEQ ID NO: 14 - DNA, 22 bp, Artificial Sequence
  K3 Reverse Primer
atgctgacagcactcggacact

SEQ ID NO: 15 - DNA, 22 bp, Artificial Sequence
  K5 Forward Primer
gctgcctacatgaacaaggtgg

SEQ ID NO: 16 - DNA, 22 bp, Artificial Sequence
  K5 Reverse Primer
atggagaggaccactgaggtgt

SEQ ID NO: 17 - DNA, 22 bp, Artificial Sequence
  K10 Forward Primer
cctgcttcagatcgacaatgcc

SEQ ID NO: 18 - DNA, 22 bp, Artificial Sequence
  K10 Reverse Primer
atctccaggtcagccttggtca

SEQ ID NO: 19 - DNA, 22 bp, Artificial Sequence
  K12 Forward Primer
agcagaatcggaaggacgctga

SEQ ID NO: 20 - DNA, 22 bp, Artificial Sequence
  K12 Reverse Primer
acctcgctcttgctggactgaa

SEQ ID NO: 21 - DNA, 22 bp, Artificial Sequence
  K14 Forward Primer
tgccgaggaatggttcttcacc

SEQ ID NO: 22 - DNA, 22 bp, Artificial Sequence
  K14 Reverse Primer
gcagctcaatctccaggttctg

SEQ ID NO: 23 - DNA, 22 bp, Artificial Sequence
  K15 Forward Primer
aggactgacctggagatgcaga

SEQ ID NO: 24 - DNA, 22 bp, Artificial Sequence
  K15 Reverse Primer
tgcgtccatctccacattgacc

SEQ ID NO: 25 - DNA, 22 bp, Artificial Sequence
  K19 Forward Primer
agctagaggtgaagatccgcga

SEQ ID NO: 26 - DNA, 22 bp, Artificial Sequence
  K19 Reverse Primer
gcaggacaatcctggagttctc

SEQ ID NO: 27 - DNA, 22 bp, Artificial Sequence
  MEIS1 Forward Primer
aagcagttggcacaagacacgg

SEQ ID NO: 28 - DNA, 22 bp, Artificial Sequence
  MEIS1 Reverse Primer
ctgctcggttggactggtctat

SEQ ID NO: 29 - DNA, 22 bp, Artificial Sequence
  MMP9 Forward Primer
gccactactgtgcctttgagtc

SEQ ID NO: 30 - DNA, 22 bp, Artificial Sequence
  MMP9 Reverse Primer
ccctcagagaatcgccagtact

SEQ ID NO: 31 - DNA, 22 bp, Artificial Sequence
  MMP10 Forward Primer
tccaggctgtatgaaggagagg

SEQ ID NO: 32 - DNA, 22 bp, Artificial Sequence
  MMP10 Reverse Primer
ggtaggcatgagccaaactgtg

SEQ ID NO: 33 - DNA, 22 bp, Artificial Sequence
  NR2F2 Forward Primer
tgcacgttgactcagccgagta

SEQ ID NO: 34 - DNA, 23 bp, Artificial Sequence
  NR2F2 Reverse Primer
aagcacactgagacttttcctgc

SEQ ID NO: 35 - DNA, 22 bp, Artificial Sequence
  NOTCH1 Forward Primer
ggtgaactgctctgaggagatc

SEQ ID NO: 36 - DNA, 22 bp, Artificial Sequence
  NOTCH1 Reverse Primer
ggattgcagtcgtccacgttga

SEQ ID NO: 37 - DNA, 22 bp, Artificial Sequence
  NOTCH3 Forward Primer
tactggtagccactgtgagcag

SEQ ID NO: 38 - DNA, 23 bp, Artificial Sequence
  NOTCH3 Reverse Primer
cagttatcaccattgtagccagg

SEQ ID NO: 39 - DNA, 22 bp, Artificial Sequence
  ODZ3 Forward Primer
ggacaaggctatcacagtggac

SEQ ID NO: 40 - DNA, 22 bp, Artificial Sequence
  ODZ3 Reverse Primer
ttctgagggagccgtcataacc

SEQ ID NO: 41 - DNA, 20 bp, Artificial Sequence
  PAX6 Forward Primer
tgtccaacggatgtgtgagt

SEQ ID NO: 42 - DNA, 20 bp, Artificial Sequence
  PAX6 Reverse Primer
tttcccaagcaaagatggac

SEQ ID NO: 43 - DNA, 22 bp, Artificial Sequence
  PDGFA Forward Primer
cagcgactcctggagatagact

SEQ ID NO: 44 - DNA, 23 bp, Artificial Sequence
  PDGFA Reverse Primer
cgatgcttctcttcctccgaatg

SEQ ID NO: 45 - DNA, 22 bp, Artificial Sequence
  PPARG Forward Primer
agcctgcgaaagccttttggtg

SEQ ID NO: 46 - DNA, 23 bp, Artificial Sequence
  PPARG Reverse Primer
ggcttcacattcagcaaacctgg

SEQ ID NO: 47 - DNA, 22 bp, Artificial Sequence
  PRDM8 Forward Primer
ctgtgtcctgagccatacttcc

SEQ ID NO: 48 - DNA, 23 bp, Artificial Sequence
  PRDM8 Reverse Primer
ccttctgaggaaccatttgctgc

SEQ ID NO: 49 - DNA, 22 bp, Artificial Sequence
  TGFB1 Forward Primer
aggactgacggagaccctcaac

SEQ ID NO: 50 - DNA, 23 bp, Artificial Sequence
  TGFB1 Reverse Primer
tccgctaaccaggatttcatcac

SEQ ID NO: 51 - DNA, 19 bp, Artificial Sequence
  WNT7A Forward Primer
tgcccggactctcatgaac

SEQ ID NO: 52 - DNA, 20 bp, Artificial Sequence
  WNT7A Reverse Primer
gtgtggtccagcacgtcttg

SEQ ID NO: 53 - DNA, 23 bp, Artificial Sequence
  Rabbit GAPDH Forward Primer
gcgagatcccgccaacatcaagt

SEQ ID NO: 54 - DNA, 20 bp, Artificial Sequence
  Rabbit GAPDH Reverse Primer
aggatgcgttgctgacaatc

SEQ ID NO: 55 - DNA, 21 bp, Artificial Sequence
  Rabbit PAX6 Forward Primer
gtattcttgcttcaggtagat

SEQ ID NO: 56 - DNA, 23 bp, Artificial Sequence
  Rabbit PAX6 Reverse Primer
gaggctcaaatgcgacttcagct

SEQ ID NO: 57 - DNA, 74 bp, Artificial Sequence
  PAX6 Transduction Forward Primer
ttcccgaattctgcagacccatgcagatgcaaaagtccaagtgctggacaatcaaaacgtgtccaacggatgtg

SEQ ID NO: 58 - DNA, 74 bp, Artificial Sequence
  PAX6 Transduction Reverse Primer
cacatccgttggacacgttttgattgtccagcacttggacttttgcatctgcatgggtctgcagaattcgggaa

SEQ ID NO: 59 - DNA, 21 bp, Artificial Sequence
  PAX6 ShRNA knockdown sequence
cgtccatctttgcttgggaaa

SEQ ID NO: 60 - DNA, 21 bp, Artificial Sequence
  PAX6 ShRNA knockdown sequence
agtttgagagaacccattatc

SEQ ID NO: 61 - DNA, 21 bp, Artificial Sequence
  WNT7A ShRNA knockdown sequence
cgtgctcaaggacaagtacaa

SEQ ID NO: 62 - DNA, 21 bp, Artificial Sequence
  WNT7A ShRNA knockdown sequence
gcgttcacctacgccatcatt

SEQ ID NO: 63 - DNA, 21 bp, Artificial Sequence
  FZD5 ShRNA knockdown sequence
cgcgagcccttcgtgcccatt

SEQ ID NO: 64 - DNA, 21 bp, Artificial Sequence
  FZD5 ShRNA knockdown sequence
tcctaaggttggcgttgtaat